(19) United States
(12) Patent Application Publication
(10) Pub. No.: US 2016/0304917 A1
(43) Pub. Date: Oct. 20, 2016

(54) Modified Microorganism for Improved Production of Alanine

(71) Applicant: BASF SE, Ludwigshafen (DE)

(72) Inventors: Joanna Martyna Krawczyk, Mannheim (DE); Stefan Haefner, Speyer (DE); Hartwig Schröder, Nußloch (DE); Oskar Zelder, Speyer (DE); Jonathan Thomas Fabarius, Reutlingen (DE)

(73) Assignee: BASF SE, Ludwigshafen (DE)

(21) Appl. No.: 14/914,855

(22) PCT Filed: Aug. 18, 2014
(86) PCT No.: PCT/IB2014/063950

§ 371 Date: Feb. 26, 2016

(30) Foreign Application Priority Data
Aug. 30, 2013 (EP) 13182425.2

(51) Int. Cl. C12P 13/06; C12N 9/06

(52) U.S. Cl. CPC C12P 13/06; C12Y 104/01001; C12N 9/0016

(57) ABSTRACT

The present invention relates to a modified microorganism having, compared to its wildtype, an increased activity of the enzyme that is encoded by the alaD-gene. The present invention also relates to a method for producing an alanine and to the use of modified microorganisms.

This application claims priority to European Patent application 13182425.2 filed on 30 Aug. 2013, which is incorporated herein by reference in its entirety.

The present invention relates to a modified microorganism from the family of Pasteurellaceae having an increased expression and/or increased activity of the enzyme alanine dehydrogenase that is encoded by the alaD-gene, to a method for producing alanine and to the use of modified microorganisms.

Amino acids are organic compounds with a carboxy-group and an amino-group. The most important amino acids are the alpha-amino acids where the amino group is located next to the carboxy group. Proteins are based on alpha-amino acids. Nine of the alpha-amino acids are essential amino acids which can not be produced by mammals and needs to be supplied with feed and food. L-alanine can be produced by fermentation with Coryneform bacterias (Hermann, 2003: Industrial production of amino acids by Coryneform bacteria, J. of Biotechnol, 104, 155-172.) or E. coli. (Zhang et al, Production of L-alanine by metabolically engineered Escheria coli. (2007) Appl. Microbiol Biotechnol., 77:355-366). L-Alanine is used in the pharmaceutical industry, veternar medicine and sweetner.

Alanin has drawn considerable interest because it has been used as an additive in the food, feed and pharmaceutical industries.

The industrial production of alanine by E. coli strains is applicable for chemical products. E. coli is containing lipopolysachharide which can elicit strong immune responses. Therefore use of E. coli to prepare material for human consumption and or pharmaceutical applications such as infusion solutions is somewhat disfavoured. It is therefore preferred to use bacterial strains for the production of feed and food products which are not derived from a former human-pathogenic organism. Such an organism is the non-pathogenic genus Basfia.

The industrial production of alanine by Coryneform bacterias is less efficient because Corynebacterium is not capable to grow under anaerobic conditions and has a very low productivity of alanin per g of biomass. Yamamoto et al. Applied and environmental microbiology; 78(12); 4447-4457 show that aerobically grown cells which grow to high density and are subsequently upconcentrated by a factor of 8.3 which are then anaerobically incubated with glucose. However, since the two different phases for the growth and production of alanine are needed in C. glutamicum the process is complex and technically challenging.

Uhlenbusch, et al. (Applied and Environmental Microbiology Volume: 57 1360-1366, 1991) show that the organisms Zymomonas mobilis is capable of producing alanine after transformation with and overexpression of an alanine dehydrogenase, however with low efficiency in only to two amounts (7.5 g/l in 25 h). It was found that a competition between alanine synthesis and ethanol production occurred. Production of alanine was also shown in recombinant Lactococcus lactis, however yield productivity and usability was found to be limited (Nature Biotechnology, Volume: 17, 588-592, 1999).

One drawback in some organisms like lactococcus lactis is that alanine can be degraded to unwanted side products such as diacetyl and acetoin which decrease the yield (Journal of Applied Microbiology, Volume: 104, 171-177, 2008).

It is an object of the present invention to provide microorganisms which can be used for the fermentative production of alanine which preferably lack the above disadvantages.

A contribution to achieving the above mentioned aim is provided by a modified microorganism of the family of Pasteurellaceae having, compared to its wildtype, an increased expression and/or activity of the enzyme that is encoded by the alanine dehydrogenase gene. The alanine dehydrogenase gene is hereinafter also referred to as alaD-gene.

Surprisingly, it has been discovered that an increase of the expression and/or activity of the enzyme that is encoded by the alaD-gene results in a recombinant Pasteurellaceae-strain that, compared to the corresponding microorganism in which the expression and/or activity of this enzyme has not been increased, is characterized by an increased yield of alanine. In contrast thereto WO2009/024294 Basfia succinici producens is described producing succinic acid.

A “wildtype” of a microorganism refers to a microorganism whose genome is present in a state as before the introduction of a genetic modification of a certain gene, e.g. alaD-gene, ldhA-gene, pflD-gene, pflA-gene and/or pckA-gene. The genetic modification may be e.g. an insertion of said gene into the genome as e.g. for alaD-gene. The genetic modification may be e.g. a deletion of a gene or a part thereof or a point mutation, e.g. ldhA-gene, pflD-gene, pflA-gene and/or pckA-gene.

The term “modified microorganism” thus includes a microorganism which has been genetically modified such that it exhibits an altered or different genotype and/or phenotype (e. g. when the genetic modification affects coding nucleic acid sequences of the microorganism) as compared to the wildtype microorganism from which it was derived. According to a particular preferred embodiment according to the present invention the modified microorganism is a recombinant microorganism, which means that the microorganism comprises at least one recombinant DNA molecule. According to a particular preferred embodiment according to the present invention the modified microorganism may be obtained by introducing point mutations.

The term “recombinant” with respect to DNA refers to DNA molecules produced by man using recombinant DNA techniques. The term comprises DNA molecules which as such do not exist in nature but are modified, changed, mutated or otherwise manipulated by man. Preferably, a “recombinant DNA molecule” is a non-naturally occurring nucleic acid molecule that differs in sequence from a naturally occurring nucleic acid molecule by at least one nucleic acid. A “recombinant DNA molecule” may also comprise a “recombinant construct” which comprises, preferably operably linked, a sequence of nucleic acid molecules not naturally occurring in that order. Preferred methods for producing said recombinant DNA molecule may comprise cloning techniques, directed or non-directed mutagenesis, gene synthesis or recombination techniques. An example of such a recombinant DNA is a plasmid into which a heterologous DNA-sequence has been inserted.

The term “expression” or “gene expression” means the transcription of a specific gene(s) or specific genetic vector construct. The term “expression” or “gene expression” in particular means the transcription of gene(s) or genetic vetor construct into mRNA. The process includes transcription of DNA and processing the resulting RNA-product. The term “expression” or “gene expression” may also include the translation of the mRNA and therewith the synthesis of the encoded protein, i.e. protein expression.

The wildtype from which the miccorganims according to the present invention are derived belongs to the family of Pasteurellaceae. Pasteurellaceae comprise a large of Gram-negative Proteobacteria with members ranging from bacteria such as Haemophilus influenzae to commensals of the animal and human mucosa. Most members live as commensals on mucosal surfaces of birds and mammals, especially in the upper respiratory tract. Pasteurellaceae are typically rod-shaped, and are a notable group of facultative anaerobes. They can be distinguished from the related Enterobacteriaceae by the presence of oxidase, and from most other similar bacteria by the absence of flagella. Bacteria in the family Pasteurellaceae have been classified into a number of genera based on metabolic properties and there sequences of the 16S RNA and 23S RNA. Many of the Pasteurellaceae contain pyruvate-formate-lyase genes and are capable of anaerobically fermenting carbon sources to organic acids. A genus of the family Pasteurellacea is the genus of Basfia, a non pathogenic group of organisms is described in Kuhnert et al. International Journal of Systematic and Evolutionary Microbiology, Volume: 60, 44-50 (2010).

According to a particular preferred embodiment of the modified microorganism according to the present invention the wildtype from which the modified microorganism has been derived belongs to the genus Basfia and it is particularly preferred that the wildtype from which the modified microorganism has been derived belongs to the species Basfia succiniciproducens.

Most preferably, the wildtype from which the modified microorganism according to the present invention as been derived is Basfia succiniciproducens-strain DD1 deposited under the Budapest Treaty with DSMZ (Deutsche Sammlung von Mikroorganismen and Zellkulturen, GmbH, Inhoffenstraβe 7B, 38124 Braunschweig, Germany) having the deposit number DSM 18541. This strain has been originally isolated from the rumen of a cow of German origin. Pasteurella bacteria can be isolated from the gastro-intestinal tract of animals and, preferably, mammals. The bacterial strain DD1, in particular, can be isolated from bovine rumen and is capable of utilizing glycerol (including crude glycerol) as a carbon source. A further strain of the genus Basfia that can be used for preparing the modified microorganism according to the present invention is the Basfia-strain that has been deposited under the deposit number DSM 22022 at DSMZ. Further strains of the genus Basfia that can be used for preparing the modified microorganism according to the present invention are the Basfia-strains that have been deposited under the deposit numbers CCUG 57335, CCUG 57762, CCUG 57763, CCUG 57764, CCUG 57765 and CCUG 57766 at Culture Collection, University of Goteborg (CCUG), Sweden (CCUG, Department of Clinical Bacteriology; Guldhedsgatan 10, SE-413 46 Goteborg, Box 7193, SE-402 34 Goteborg, Sweden). Said strains have been originally isolated from the rumen of cows of German or Swiss origin.

According to a preferred embodiment according to the present invention, the modified microorganism is not characterized by a sucrose-mediated catabolic repression of glycerol. Microorganisms showing a sucrose-mediated catabolic repression of glycerol are, for example, disclosed in WO-A-2012/030130.

In this context, it is particularly preferred that the wildtype from which the modified microorganism according to the present invention has been derived has a 16S rDNA of SEQ ID NO: 1 or a sequence, which shows a sequence identity of preferably at least 96%, at least 97%, at least 98%, at least 99%, at least 99.5%, at least 99.6%, at least 99.7%, at least 99.8% or at least 99.9% with SEQ ID NO: 1, the identity being the identity over the whole length of nucleic acid with SEQ ID NO:1.

In this context, it is particularly preferred that the wildtype from which the modified microorganism according to the present invention has been derived has a 23S rDNA of SEQ ID NO: 2 or a sequence, which shows a sequence identity preferably of at least 96%, at least 97%, at least 98%, at least 99%, at least 99.5%, at least 99.6%, at least 99.7%, at least 99.8% or most preferably at least 99.9% with SEQ ID NO: 2, the identity being the identity over the whole length of nucleic acid with SEQ ID NO:2.

The identity in percentage values referred to in connection with the various polypeptides or polynucleotides to be used for the modified microorganism according to the present invention is, preferably, calculated as identity of the residues over the complete length of the aligned sequences, such as, for example, the identity calculated (for rather similar sequences) with the aid of the program needle from the bioinformatics software package EMBOSS (Version 5.0.0, http://emboss.source-forge.net/what/) with the default parameters which are, i.e. gap open (penalty to open a gap): 10.0, gap extend (penalty to extend a gap): 0.5, and data file (scoring matrix file included in package): EDNAFUL. It should be noted that the modified microorganism according to the present invention can not only be derived from the above mentioned wildtype-microorganisms, especially from Basfia succiniciproducens-strain DD1, but also from variants of these strains. In this context the expression “a variant of a strain” comprises every strain having the same or essentially the same characteristics as the wildtype-strain. In this context it is particularly preferred that the 16 S rDNA of the variant has an identity of at least 99%, preferably at least 99.5%, at least 99.6%, at least 99.7%, at least 99.8%, at least 99.9% or most preferably at least 99.9% with the wildtype from which the variant has been derived. Furthermore, it is particularly preferred that the 23 S rDNA of the variant has an identity of at least 99%, preferably at least 99.5%, at least 99.6%, at least 99.7%, at least 99.8%, at least 99.9% or most preferably at least 99.9% with the wildtype from which the variant has been derived. A variant of a strain in the sense of this definition can, for example, be obtained by treating the wildtype-strain with a mutagenizing chemical agent, X-rays, or UV light.

The modified microorganism according to the present invention is characterized in that, compared to its wildtype, the expression and/or the activity of the enzyme that is encoded by the alaD-gene is increased. The term “increased expression and/or activity of the enzyme that is encoded by the alaD-gene”, also encompasses a wildtype microorganism which has no detectable expression and/or activity of the enzyme that is encoded by the alaD-gene. Methods for the detection and determination of the expression and/or activity of the enzyme that is encoded by the alaD-gene can be found, for example, in the Jojima T, Fujii M, Mori E, lnui M, Yukawa H., Engineering of sugar metabolism of Corynebacterium glutamicum for production of amino acid L-alanine under oxygen deprivation (2010) Appl Microbiol Biotechnol. 87, 159-165; in WO 2008119009 A2 (Materials and methods for efficient alanine production); A. Freese, E. Biochim. Biophys. Acta 96, 248-262 (1965) or Sakamoto et al., J. Ferment. Bioeng. 69, 154-158 (1990); Honorat et al. Enzyme Microb. Technol. 12, 515-520 (1990); or Laue, H.; Cook, A. M., Arch. Microbiol. 174, 162-167 (2000). Preferred is the method described in Jojima et al. (2010).

In one embodiment the increase of the expression and/or activity of alanine dehydrogenase (alaD) is an increase of the expression and/or enzymatic activity by at least 110%, compared to the expression and/or activity of said enzyme in the wildtype of the microorganism, or an increase of the expression and/or enzymatic activity by at least 120%, or more preferably an increase of expression and/or the enzymatic activity by at least 130%, or more preferably an increase of expression and/or the enzymatic activity by at least 140%, or even more preferably an increase of the expression and/or enzymatic activity by at least 150% or even more preferably an rincrease of the expression and/or the enzymatic activity by at least 160%. The expression and/or enzymatic activity of alanine dehydrogenase in the wildtype is 100% compared to the increased expression and/or enzymatic activity. The term “increased expression and/or activity of the enzyme that is encoded by the alaD-gene also may also encompasses a modified microorganism which has no detectable expression and/or activity of this enzyme.

In one embodiment the increase of the expression and/or activity of alanine dehydrogenase is achieved by an activation of the alaD-gene which encodes the alanine dehydrogenase; EC 1.4.1.1.

The alaD-gene preferably comprises a nucleic acid selected from the group consisting of:
a) nucleic acids having the nucleotide sequence of SEQ ID NO: 3;
b) nucleic acids encoding the amino acid sequence of SEQ ID NO: 4;
c) nucleic acids which are at least 80%, preferably at least 90%, more preferably at least 95% and most preferably at least 96%, most preferably at least 97%, most preferably at least 98% most preferably at least 99% identical to the nucleic acid of a) or b), the identity being the identity over the total length of the nucleic acids of a) or b); and
d) nucleic acids encoding an amino acid sequence which is at least 60%, preferably at least 70%, preferably, at least 80%, preferably at least 90%, more preferably at least 95% and most preferably at least 96%, most preferably at least 97%, most preferably at least 98%, most preferably at least 99% identical to the amino acid sequence encoded by the nucleic acid of a) or b), the identy being the identity over the total length of amino acid sequence encoded by the nucleic acids of a) or b), wherein preferably the proteins encoded by the nucleic acids as defined under b) to d) have at least 10%, preferably at least 20% at least 30%, more preferably at least 40%, at least 50%, more preferably at least 60%, more preferably at least 70%, most preferably at least 80%, most preferably at least 90%, most preferably at least 95% activitiy as the protein encoded by the nucleic acid as defined in a).

The term “increased gene expression of an enzyme” includes, for example, the expression of the enzyme by said genetically manipulated (e.g., genetically engineered) microorganism at a higher level than than expressed by the wildtype of said microorganism or de novo expression. Genetic manipulations for increasing the expression of a gene coding for an enzyme can include, but are not limited to, introducing one copy or additional copies of the corresponding gene, altering or modifying regulatory sequences or sites associated with expression of the gene encoding the enzyme (e.g., by introducing strong promoters or removing repressible promoters compared the respective wildtype), modifying proteins (e.g., regulatory proteins, suppressors, enhancers, transcriptional activators and the like) involved in transcription of the gene encoding the enzyme and/or the translation of the gene product, or any other conventional means of increasing expression of a particular gene routine in the art.

Furthermore, an increase of the activity of an enzyme may also include an activation (or the increased expression) of activating enzymes which are necessary in order to activate the enzyme the activity of which is to be increased.

According to a preferred embodiment of the modified microorganism according to the present invention, an increase of the expression and/or activity of the enzyme encoded by the alaD-gene is achieved by a modification of the alaD-gene, wherein this modification is preferably realized by an insertion of the alaD-gene into the genome of the micororganism, e.g. homologous recombination of the alaD-gene preferably in the pflD-locus of Basfia succinic producens. In the following, a suitable technique for inserting sequences is described.

According to a further preferred embodiment of the modified microorganism according to the present invention, this microorganism is not only characterized by an increased expression and/or activity of the enzyme encoded by the A/aD-gene, but also, compared to the wildtype, by

i) a reduced ldhA expression and/or activity,

ii) a reduced pflD expression and/or activity

iii) a reduced pflA expression and/or activity and/or

iv) a reduced expression and/or pckA activity.

The reduced expression and/or activity of the enzymes disclosed herein, in particular the reduced expression and/or reduced activity of the enzyme encoded by the lactate dehydrogenase (ldhA), pyruvate formate lyase (pflD), pyruvate formate lyase activator (pflA) and/or the phosphoenolpyruvate carboxylase (pckA), can be a reduction of the expression and/or enzymatic activity by at least 50%, compared to the expression and/or activity of said enzyme in the wildtype of the microorganism, or a reduction of the expression and/or enzymatic activity by at least 90%, or more preferably a reduction of expression and/or the enzymatic activity by at least 95%, or more preferably a reduction of expression and/or enzymatic activity by at least 98%, or even more preferably a reduction of the expression and/or enzymatic activity by at least 99% or even more preferably a reduction of the expression and/or the enzymatic activity by at least 99.9%. The term “reduced expression and/or activity of the enzyme that is encoded by the ldhA-gene”, “reduced activity of the enzyme that is encoded by the pflD-gene”, “reduced activity of the enzyme that is encoded by the pflA-gene” or “reduced activity of the enzyme that is encoded by the pckA-gene” also encompasses a modified microorganism which has no detectable expression and/or activity of these enzymes. Methods for the detection and determination of the expression and/or activity of the enzyme that is encoded by the said genes can be found, for example:

Methods for determining the phosphoenolpyruvate carboxylase expression or activity are, for example, disclosed in G. P. Bridger, T. K. Sundaram (1976) Occurrence of phosphenolpyruvate carboxylase in the extremely thermophilic bacterium Thermus aquaticus, J Bacteriol. 125, 1211-1213; P. Maeba, B. D. Sanwal (1969) Phosphoenolpyruvate carboxylase from Salmonella typhimurium strain LT2, Methods in Enzymology 13, 283-288; or J. L. Cánovas, H. L. Kornberg (1969) Phosphoenolpyruvate carboxylase from Escherichia coli, Methods in Enzymology 13, 288-292. Preferred is the method described in disclosed in G. P. Bridger, T. K. Sundaram (1976).

Methods for determining the lactate dehydrogenase expression or activity are, for example, disclosed by Bunch et al. in “The ldhA gene encoding the fermentative lactate de hydrogenase of Escherichia Coli”, Microbiology (1997), Vol. 143, pages 187-155; or Bergmeyer, H. U., Bergmeyer J. and Grassi, M. (1983-1986) in “Methods of Enzymatic Analysis”, 3rd Edition, Volume III, pages 126-133, Verlag Chemie, Weinheim; or Enzymes in Industry: Production and Applications, Second Edition (2004), Wolfgang Aehle, page 23. Preferred is the last method.

Methods for determining the pyruvate formate lyase expression or activity are, for example, disclosed in by Knappe and Blaschkowski in “Pyruvate formate-lyase from Escherichia coli and its activation system”, Methods Enzymol. (1975), Vol. 41, pages 508-518; or Asanuma N. and Hino T. in “Effects of pH and Energy Supply on Activity and Amount of Pyruvate-Formate-Lyase in Streptococcus bovis”, Appl. Environ. Microbiol. (2000), Vol. 66, pages 3773-3777″. Preferred is the last method.

Methods for determining the pyruvate formate-lyase activating enzyme expression or activity pyruvate formate lyase activity are disclosed by Takahashi-Abbe S., Abe K., Takahashi N., Biochemical and functional properties of a pyruvate formate-lyase (PFL)-activating system in Streptococcus mutans (2003) Oral Microbiology Immunology 18, 293-297.

The term “reduced expression of an enzyme” includes, for example, the expression of the enzyme by said genetically manipulated (e.g., genetically engineered) microorganism at a lower level than that expressed by the wildtype of said microorganism. Genetic manipulations for reducing the expression of an enzyme can include, but are not limited to, deleting the gene or parts thereof encoding for the enzyme, altering or modifying regulatory sequences or sites associated with expression of the gene encoding the enzyme (e.g., by removing strong promoters or repressible promoters), modifying proteins (e.g., regulatory proteins, suppressors, enhancers, transcriptional activators and the like) involved in transcription of the gene encoding the enzyme and/or the translation of the gene product, or any other conventional means of decreasing expression of a particular gene routine in the art (including, but not limited to, the use of antisense nucleic acid molecules or other methods to knock-out or block expression of the target protein). Further on, one may introduce destabilizing elements into the mRNA or introduce genetic modifications leading to deterioration of ribosomal binding sites (RBS) of the RNA. Further on, one may introduce antisense or RNAi-constructs into the genome leading to deterioration of the RNA. It is also possible to change the codon usage of the gene in a way, that the translation efficiency and speed is decreased.

According to a preferred embodiment of the modified microorganism according to the present invention, a reduction of the expression and/or activity of the enzyme encoded by the ldhA-gene, pflD-gene, pflA-gene and/or pckA-gene is achieved by a modification of the ldhA-gene, pflD-gene, pflA-gene and/or pckA-gene, wherein this/these gene modification(s) is(are) preferably realized by a deletion of one or more of said genes or at least a part thereof, a deletion of a regulatory element of the one or more of said genes or parts thereof, such as a promotor sequence, by a frameshift, by introducing a stop codon, by an introduction of at least one deleterious mutation into one or more of said genes. Further on, one may introduce antisense or RNAi-constructs into the genome leading to deterioration of the corresponding RNA expressed from one or more of said genes.

A reduced activity of an enzyme can also be obtained by introducing one or more deleterious gene mutations which lead to a reduced activity of the enzyme. Furthermore, a reduction of the activity of an enzyme may also include an inactivation (or the reduced expression) of activating enzymes which are necessary in order to activate the enzyme the activity of which is to be reduced. By the latter approach the enzyme the activity of which is to be reduced is preferably kept in an inactivated state.

A deleterious mutation may be any mutation within a gene comprising promoter and coding region that lead to a decreased or deleted protein activity of the protein encoded by the coding region of the gene. Such deleterious mutations comprise for example frameshifts, introduction of stop-codons in the coding region, mutation of promoter elements such as the TATA box that prevent transcription and the like.

Microorganisms having a reduced expression and/or activity of the enzyme encoded by the ldhA-gene, pflD-gene, pflA-gene and/or pckA-gene may occur naturally, i.e. due to spontaneous deleterious mutations. A microorganism can be modified to lack or to have significantly reduced activity of the enzyme that is encoded by one or more of said genes by various techniques, such as chemical treatment or radiation. To this end, microorganisms will be treated by, e.g., a mutagenizing chemical agent, X-rays, or UV light. In a subsequent step, those microorganisms which have a reduced expression and/or activity of the enzyme that is encoded by one or more of said genes will be selected. Modified microorganisms are also obtainable by homologous recombination techniques which aim to mutate, disrupt or excise one or more of said genes in the genome of the microorganism or to substitute one or more of said genes with a corresponding gene that encodes for an enzyme which, compared to the enzyme encoded by the wildtype-gene, has a reduced expression and/or activity.

A mutation into the above-gene can be introduced, for example, by site-directed or random mutagenesis, followed by an introduction of the modified gene into the genome of the microorganism by recombination. Variants of the genes can be are generated by mutating the gene sequences by means of PCR. The “Quickchange Site-directed Mutagenesis Kit” (Stratagene) can be used to carry out a directed mutagenesis. A random mutagenesis over the entire coding sequence, or else only part thereof, can be performed with the aid of the “GeneMorph II Random Mutagenesis Kit” (Stratagene). The mutagenesis rate is set to the desired amount of mutations via the amount of the template DNA used. Multiple mutations are generated by the targeted combination of individual mutations or by the sequential performance of several mutagenesis cycles.

In the following, a suitable technique for recombination, in particular for introducing a mutation or for deleting sequences, is described.

This technique is also sometimes referred to as the “Campbell recombination” herein (Leenhouts et al., Appl Env Microbiol. (1989), Vol. 55, pages 394-400). “Campbell in”, as used herein, refers to a transformant of an original host cell in which an entire circular double stranded DNA molecule (for example a plasmid) has integrated into a chromosome by a single homologous recombination event (a cross in event), and that effectively results in the insertion of a linearized version of said circular DNA molecule into a first DNA sequence of the chromosome that is homologous to a first DNA sequence of the said circular DNA molecule. “Campbelled in” refers to the linearized DNA sequence that has been integrated into the chromosome of a “Campbell in” transformant. A “Campbell in” contains a duplication of the first homologous DNA sequence, each copy of which includes and surrounds a copy of the homologous recombination crossover point.

“Campbell out”, as used herein, refers to a cell descending from a “Campbell in” transformant, in which a second homologous recombination event (a cross out event) has occurred between a second DNA sequence that is contained on the linearized inserted DNA of the “Campbelled in” DNA, and a second DNA sequence of chromosomal origin, which is homologous to the second DNA sequence of said linearized insert, the second recombination event resulting in the deletion (jettisoning) of a portion of the integrated DNA sequence, but, importantly, also resulting in a portion (this can be as little as a single base) of the integrated Campbelled in DNA remaining in the chromosome, such that compared to the original host cell, the “Campbell out” cell contains one or more intentional changes in the chromosome (for example, a single base substitution, multiple base substitutions, insertion of a heterologous gene or DNA sequence, insertion of an additional copy or copies of a homologous gene or a modified homologous gene, or insertion of a DNA sequence comprising more than one of these aforementioned examples listed above). A “Campbell out” cell is, preferably, obtained by a counter-selection against a gene that is contained in a portion (the portion that is desired to be jettisoned) of the “Campbelled in” DNA sequence, for example the Bacillus subtilis sacB-gene, which is lethal when expressed in a cell that is grown in the presence of about 5% to 10% sucrose. Either with or without a counter-selection, a desired “Campbell out” cell can be obtained or identified by screening for the desired cell, using any screenable phenotype, such as, but not limited to, colony morphology, colony color, presence or absence of antibiotic resistance, presence or absence of a given DNA sequence by polymerase chain reaction, presence or absence of an auxotrophy, presence or absence of an enzyme, colony nucleic acid hybridization, antibody screening, etc. The term “Campbell in” and “Campbell out” can also be used as verbs in various tenses to refer to the method or process described above.

It is understood that the homologous recombination events that leads to a “Campbell in” or “Campbell out” can occur over a range of DNA bases within the homologous DNA sequence, and since the homologous sequences will be identical to each other for at least part of this range, it is not usually possible to specify exactly where the crossover event occurred. In other words, it is not possible to specify precisely which sequence was originally from the inserted DNA, and which was originally from the chromosomal DNA. Moreover, the first homologous DNA sequence and the second homologous DNA sequence are usually separated by a region of partial non-homology, and it is this region of non-homology that remains deposited in a chromosome of the “Campbell out” cell.

Preferably, first and second homologous DNA sequence are at least about 200 base pairs in length, and can be up to several thousand base pairs in length. However, the procedure can be made to work with shorter or longer sequences. For example, a length for the first and second homologous sequences can range from about 500 to 2000 bases, and the obtaining of a “Campbell out” from a “Campbell in” is facilitated by arranging the first and second homologous sequences to be approximately the same length, preferably with a difference of less than 200 base pairs and most preferably with the shorter of the two being at least 70% of the length of the longer in base pairs.

In one embodiment the increase of the activity of alanine dehydrogenase is achieved by an increased expression and/or activation of the alaD-gene preferably by means of the “Campbell recombination” as described above.

In one embodiment the reduction of the expression and/or activity of lactate dehydrogenase is achieved by an inactivation of the ldhA-gene which encodes the lactate dehydrogenase EC 1.1.1.27 or EC 1.1.1.28, the reduction of the expression and/or activity of the pyruvate formate lyase is achieved by an inactivation of the pflA-gene which encodes for an activator of pyruvate formate lyase EC 1.97.1.4 or the reduction of the expression and/or activity of the pyruvate formate lyase is achieved by an inactivation the pflD-gene which encodes the pyruvate formate lyase EC 2.3.1.54 and/or the reduction of the expression and/or activity of the phosphoenolpyruvate carboxylase is achieved by an inactivation of the pckA-gene which encodes the phosphoenolpyruvate carboxylase EC 4.1.1.49.

In one embodiment the inactivation of these genes (i. e. ldhA, pflA, pflD and/or pckA) is preferably achieved by a deletion of theses genes or parts thereof, by a deletion of a regulatory element of these genes or at least a part thereof or by an introduction of at least one deleterious mutation into these genes, wherein these modifications are preferably performed by means of the “Campbell recombination” as described above.

The ldhA-gene preferably comprises a nucleic acid selected from the group consisting of:
a) nucleic acids having the nucleotide sequence of SEQ ID NO: 5;
b) nucleic acids encoding the amino acid sequence of SEQ ID NO: 6;
c) nucleic acids which are at least 80%, preferably at least 90%, more preferably at least 95% and most preferably at least 96%, most preferably at least 97%, most preferably at least 98% most preferably at least 99% identical to the nucleic acid of a) or b), the identity being the identity over the total length of the nucleic acids of a) or b); and
d) nucleic acids encoding an amino acid sequence which is at least 80%, preferably at least 90%, more preferably at least 95% and most preferably at least 96%, most preferably at least 97%, most preferably at least 98%, most preferably at least 99% identical to the amino acid sequence encoded by the nucleic acid of a) or b), the identy being the identity over the total length of amino acid sequence encoded by the nucleic acids of a) or b).

The pflD-gene preferably comprises a nucleic acid selected from the group consisting of:
a) nucleic acid having the nucleotide sequence of SEQ ID NO: 7;
b) nucleic acid encoding the amino acid sequence of SEQ ID NO: 8;
c) nucleic acids which are at least 80%, preferably at least 90%, more preferably at least 95% and most preferably at least 96%, most preferably at least 97%, most preferably at least 98% most preferably at least 99% identical to the nucleic acid of a) or b), the identity being the identity over the total length of the nucleic acids of a) or b); and
d) nucleic acids encoding an amino acid sequence which is at least 80%, preferably at least 90%, more preferably at least 95% and most preferably at least 96%, most preferably at least 97%, most preferably at least 98%, most preferably at least 99% identical to the amino acid sequence encoded by the nucleic acid of a) or b), the identy being the identity over the total length of amino acid sequence encoded by the nucleic acids of a) or b).

Modified microorganisms being deficient in lactate dehydrogenase and/or being deficient in pyruvate formate lyase activity are disclosed in WO-A-2010/092155, US 2010/0159543 and WO-A-2005/052135, the disclosure of which with respect to the different approaches of reducing the activity of lactate dehydrogenase and/or pyruvate formate lyase in a microorganism, preferably in a bacterial cell of the genus Pasteurella, particular preferred in Basfia succiniciproducens strain DD1, is incorporated herein by reference.

The pflA-gene preferably comprises a nucleic acid selected from the group consisting of:
a) nucleic acid having the nucleotide sequence of SEQ ID NO: 9;
b) nucleic acid encoding the amino acid sequence of SEQ ID NO: 10;
c) nucleic acids which are at least 80%, preferably at least 90%, more preferably at least 95% and most preferably at least 96%, most preferably at least 97%, most preferably at least 98% most preferably at least 99% identical to the nucleic acid of a) or b), the identity being the identity over the total length of the nucleic acids of a) or b); and
d) nucleic acids encoding an amino acid sequence which is at least 80%, preferably at least 90%, more preferably at least 95% and most preferably at least 96%, most preferably at least 97%, most preferably at least 98%, most preferably at least 99% identical to the amino acid sequence encoded by the nucleic acid of a) or b), the identy being the identity over the total length of amino acid sequence encoded by the nucleic acids of a) or b).

The pckA-gene preferably comprises a nucleic acid selected from the group consisting of:
a) nucleic acid having the nucleotide sequence of SEQ ID NO: 11;
b) nucleic acid encoding the amino acid sequence of SEQ ID NO: 12;
c) nucleic acids which are at least 80%, preferably at least 90%, more preferably at least 95% and most preferably at least 96%, most preferably at least 97%, most preferably at least 98% most preferably at least 99% identical to the nucleic acid of a) or b), the identity being the identity over the total length of the nucleic acids of a) or b); and
d) nucleic acids encoding an amino acid sequence which is at least 80%, preferably at least 90%, more preferably at least 95% and most preferably at least 96%, most preferably at least 97%, most preferably at least 98%, most preferably at least 99% identical to the amino acid sequence encoded by the nucleic acid of a) or b), the identy being the identity over the total length of amino acid sequence encoded by the nucleic acids of a) or b).

In this context, it is preferred that the modified microorganism according to the present invention comprises
a) an insertion of the alaD-gene,
b) a deletion of the pflD-gene or at least a part thereof, a deletion of a regulatory element of the pflD-gene or at least a part thereof or an introduction of at least one deleterious mutation into the pflD-gene or a deletion of the pflA-gene or at least a part thereof, a deletion of a regulatory element of the pflA-gene or at least a part thereof or an introduction of at least one deleterious mutation into the pflA-gene; and
c) a deletion of the pckA-gene or at least a part thereof, a deletion of a regulatory element of the pckA-gene or at least a part thereof or an introduction of at least one deleterious mutation into the pckA-gene.

A contribution to solving the problems mentioned at the outset is furthermore provided by a method of producing an organic compound comprising:
I) cultivating the modified microorganism according to the present invention under suitable culture conditions in a culture medium an assimilable carbon source to allow the modified microorganism to produce alanine, thereby obtaining a fermentation broth comprising alanine;
II) recovering the alanine from the fermentation broth obtained in process step I).

The term “alanine”, as used in the context of the present invention, has to be understood in its broadest sense and also encompasses salts thereof, as for example alkali metal salts, like Na⁺ and K⁺-salts, or earth alkali salts, like Mg²⁺ and Ca²⁺-salts, or ammonium salts or anhydrides of alanine.

The modified microorganism according to the present invention is, preferably, incubated in the culture medium at a temperature in the range of about 10 to 60° C. or 20 to 50° C. or 30 to 45° C. at a pH of 5.0 to 9.0 or 5.5 to 8.0 or 6.0 to 7.0.

Preferably, alanine is produced under anaerobic conditions. Aerobic or micoraerobic conditions may be also used. Anaerobic conditions may be established by means of conventional techniques, as for example by degassing the constituents of the reaction medium and maintaining anaerobic conditions by introducing carbon dioxide or nitrogen or mixtures thereof and optionally hydrogen at a flow rate of, for example, 0.1 to 1 or 0.2 to 0.5 vvm. Aerobic conditions may be established by means of conventional techniques, as for example by introducing air or oxygen at a flow rate of, for example, 0.1 to 1 or 0.2 to 0.5 vvm. If appropriate, a slight over pressure of 0.1 to 1.5 bar may be applied in the process.

According to one embodiment microaerobic means that the concentration of oxygen is less than that in air. According to one embodiment microaerobic means oxygen tension between 5 and 27 mm Hg, preferably between 10 and 20 Hg (Megan Falsetta et al. (2011), The composition and metabolic phenotype of Neisseria gonorrhoeae biofilms, Frontiers in Microbiology, Vol 2, page 1 to 11).

According to one embodiment of the process according to the present invention the assimilable carbon source may be glucose, glycerin, glucose, maltose, maltodextrin, fructose, galactose, mannose, xylose, sucrose, arabinose, lactose, raffinose and combinations thereof.

In a preferred embodiment the assimiable carbon source is glucose, sucrose, xylose, arabinose, glycerol or combinations thereof. Preferred carbon sources are

glucose,

sucrose,

glucose and sucrose,

glucose and xylose and/or

glucose, arabinose and xylose.

According to one embodiment of the process according to the present invention the assimilable carbon source may be glucose, glycerin and/or glucose.

The initial concentration of the assimilable carbon source, preferably the initial concentration is, preferably, adjusted to a value in a range of 5 to 100 g/l, preferably 5 to 75 g/l and more preferably 5 to 50 g/l and may be maintained in said range during cultivation. The pH of the reaction medium may be controlled by addition of suitable bases as for example, gaseous ammonia, NH₄OH, NH₄HCO₃, (NH₄)₂CO₃, NaOH, Na₂OC₃, NaHCO₃, KOH, K₂CO₃, KHCO₃, Mg(OH)₂, MgCO₃, Mg(HCO₃)₂, Ca(OH)₂, CaCO₃, Ca(HCO₃)₂, CaO, CH₆N₂O₂, C₂H₇N and/or mixtures thereof.

The fermentation step I) according to the present invention can, for example, be performed in stirred fermenters, bubble columns and loop reactors. A comprehensive overview of the possible method types including stirrer types and geometric designs can be found in Chmiel: “Bioprozesstechnik: Einführung in die Bioverfahrenstechnik”, Volume 1. In the process according to the present invention, typical variants available are the following variants known to those skilled in the art or explained, for example, in Chmiel, Hammes and Bailey: “Biochemical Engineering”, such as batch, fed-batch, repeated fed-batch or else continuous fermentation with and without recycling of the biomass. Depending on the production strain, sparging with air, oxygen, carbon dioxide, hydrogen, nitrogen or appropriate gas mixtures may be effected in order to achieve good yield (YP/S). Particularly preferred conditions for producing alanine in process step I) are:

Assimilable carbon source: glucose

Temperature: 30 to 45° C.

pH: 5.5 to 7.0

Supplied gas: gaseous ammonia

In process step II) alanine is recovered from the fermentation broth obtained in process step I).

Usually, the recovery process comprises the step of separating the recombinant microrganims from the fermentation broth as the so called “biomass”. Processes for removing the biomass are known to those skilled in the art, and comprise filtration, sedimentation, flotation or combinations thereof. Consequently, the biomass can be removed, for example, with centrifuges, separators, decanters, filters or in a flotation apparatus. For maximum recovery of the product of value, washing of the biomass is often advisable, for example in the form of a diafiltration. The selection of the method is dependent upon the biomass content in the fermentation broth and the properties of the biomass, and also the interaction of the biomass with the organic compound (e. the product of value). In one embodiment, the fermentation broth can be sterilized or pasteurized. In a further embodiment, the fermentation broth is concentrated. Depending on the requirement, this concentration can be done batch wise or continuously. The pressure and temperature range should be selected such that firstly no product damage occurs, and secondly minimal use of apparatus and energy is necessary. The skillful selection of pressure and temperature levels for a multistage evaporation in particular enables saving of energy.

The recovery process may further comprise additional purification steps in which alanine is further purified. If, however, alanine is converted into a secondary organic product by chemical reactions as described below, a further purification of alanine is, depending on the kind of reaction and the reaction conditions, not necessarily required. For the purification of alanine obtained in process step II) methods known to the person skilled in the art can be used, as for example crystallization, filtration, electrodialysis and chromatography. The resulting solution may be further purified by means of ion exchange chromatography in order to remove undesired residual ions.

According to a preferred embodiment of the process according to the present invention the process further comprises the process step:
III) conversion alanine contained in the fermentation broth obtained in process step I) or conversion of the recovered organic compound obtained in process step II) into a secondary organic product being different from the organic compound by at least one chemical reaction.

The invention is now explained in more detail with the aid of figures and non-limiting examples.

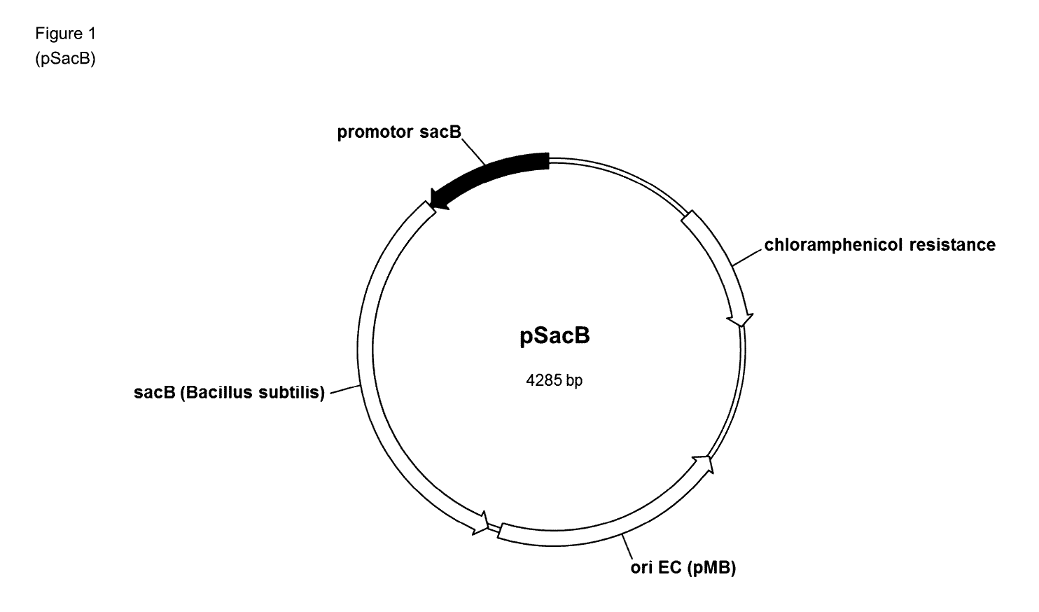

FIG. 1 shows a schematic map of plasmid pSacB.

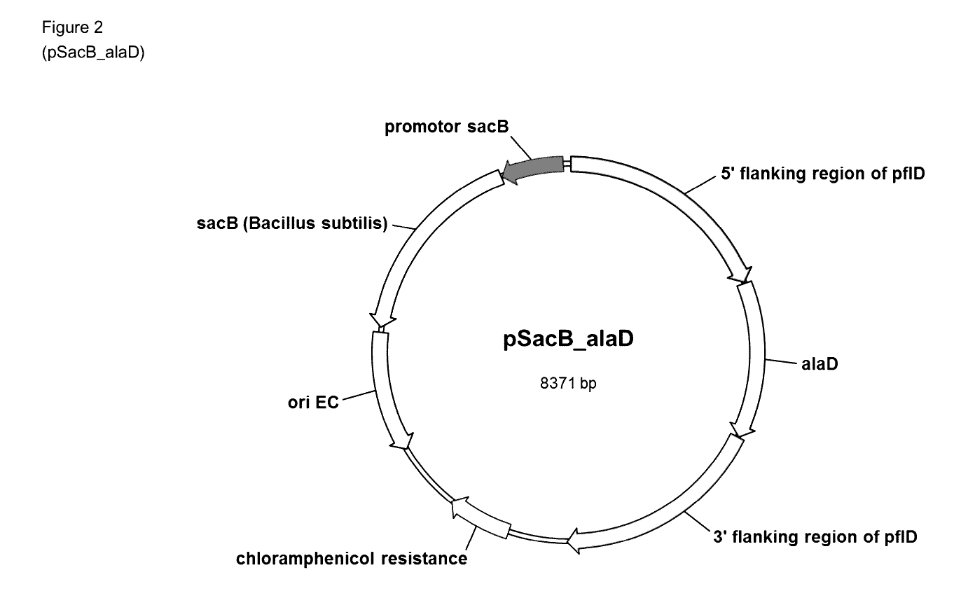

FIG. 2 shows a schematic map of plasmid pSacB alaD.

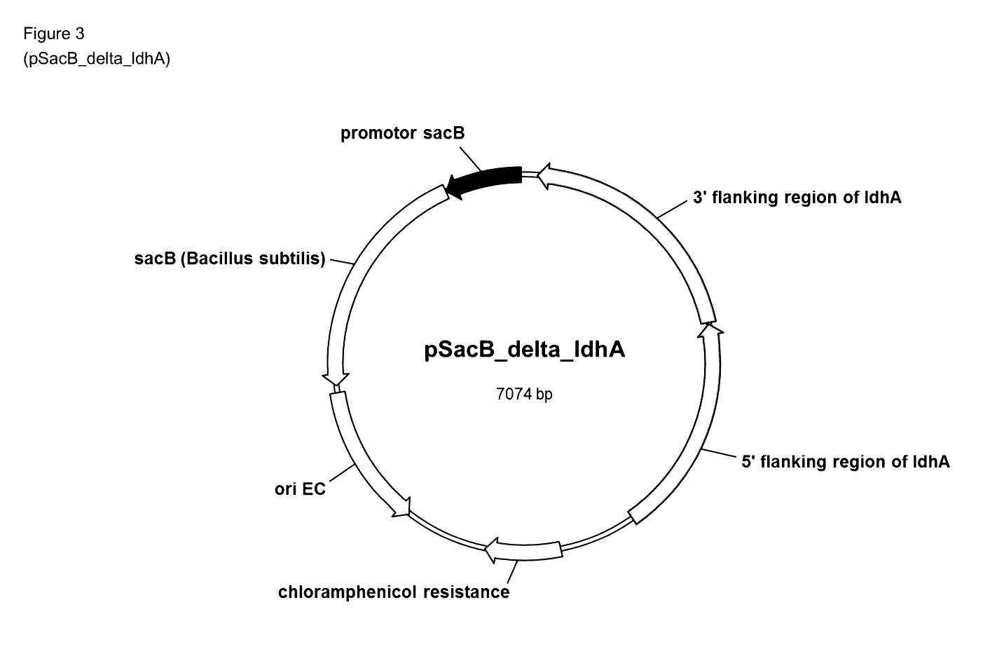

FIG. 3 shows a schematic map of plasmid pSacB ΔldhA.

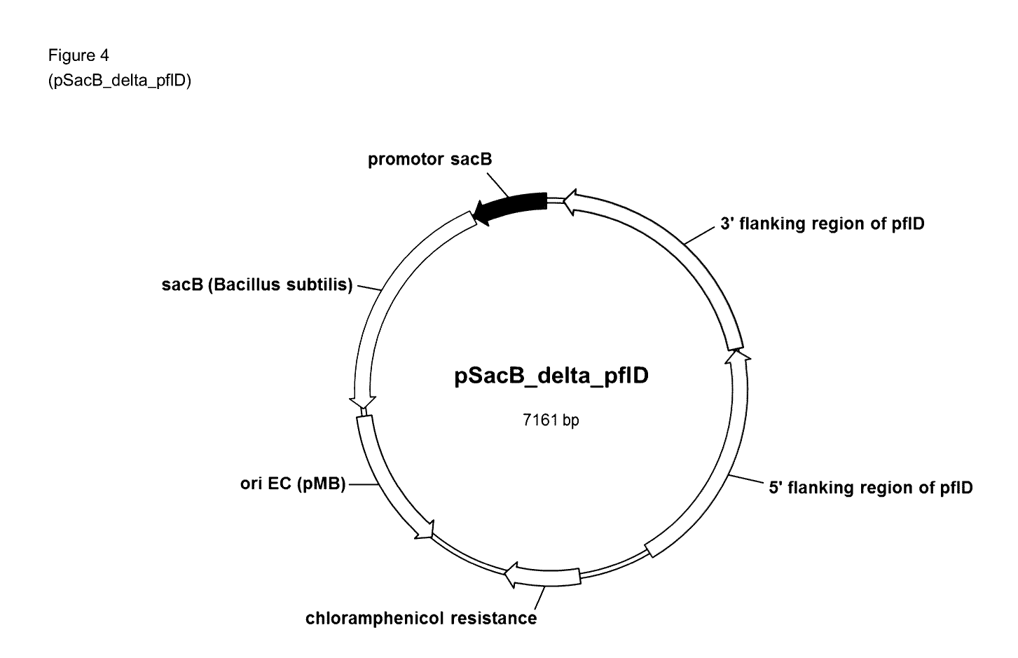

FIG. 4 shows a schematic map of plasmid pSacB ΔpflD.

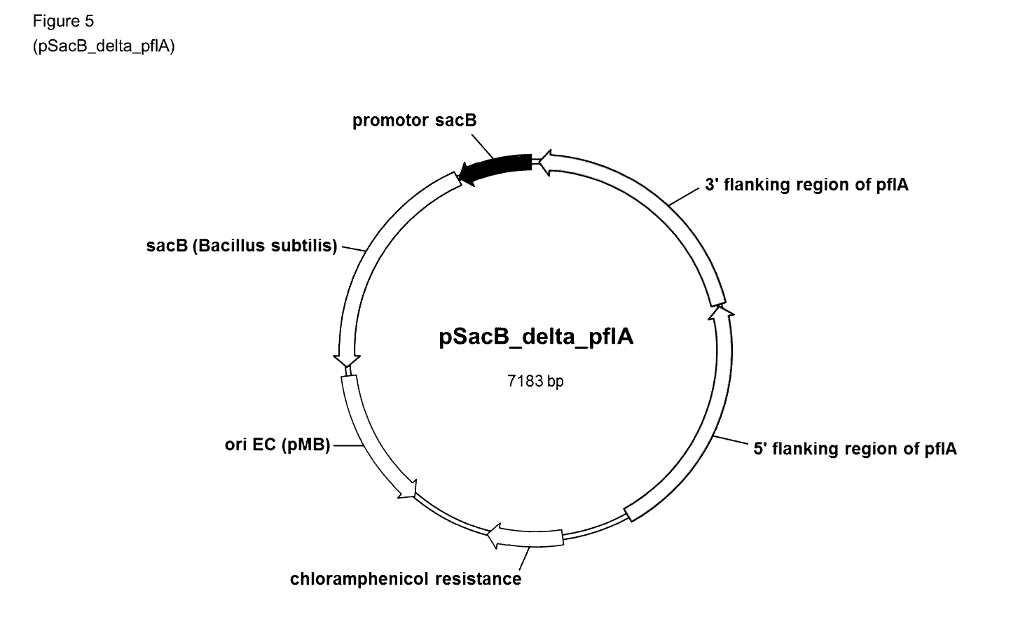

FIG. 5 shows a schematic map of plasmid pSacB ΔpflA.

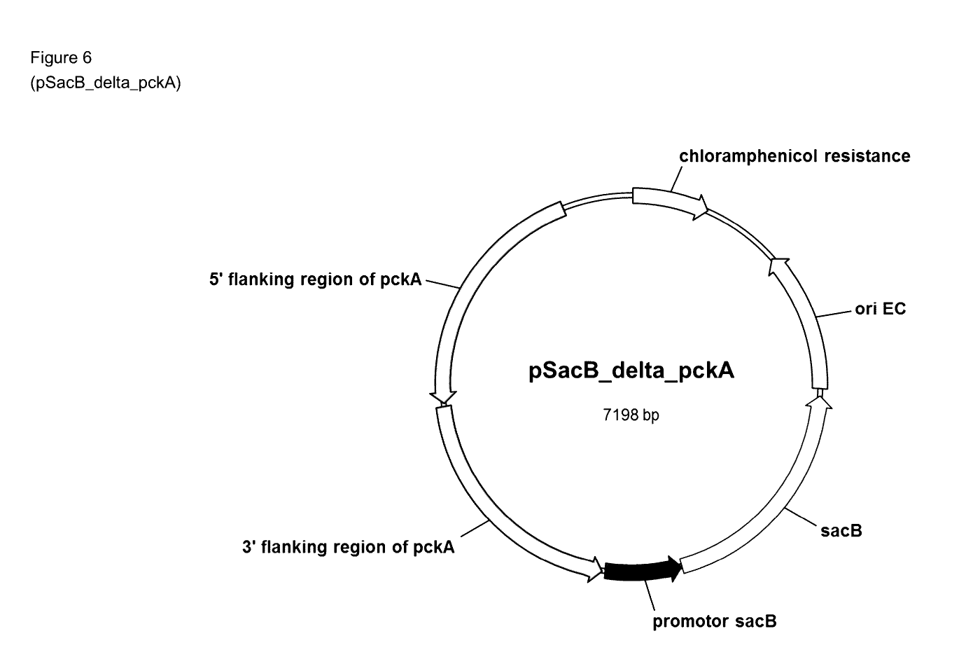

FIG. 6 shows a schematic map of plasmid pSacB ΔpckA.

### EXAMPLES

### Example 1

### General Method for the Transformation of Basfia succiniciproducens

TABLE 1
Nomenclature of the DD1-wildtype and
mutants referred to in the examples
Strain
Wildtype DD1 (deposit DSM18541)
DD1 ΔldhA ΔpflD (DD3)
DD1 ΔldhA ΔpflD alaD (DD3 alaD)
DD1 ΔldhA ΔpflD ΔpckA alaD (DD3 ΔpckA alaD)

Basfia succiniciproducens DD1 (wildtype) was transformed with DNA by electroporation using the following protocol:

For preparing a pre-culture DD1 was inoculated from frozen stock into 40 ml BHI (brain heart infusion; Becton, Dickinson and Company) in 100 ml shake flask. Incubation was performed over night at 37° C.; 200 rpm. For preparing the main-culture 100 ml BHI were placed in a 250 ml shake flask and inoculated to a final OD (600 nm) of 0.2 with the pre-culture. Incubation was performed at 37° C., 200 rpm. The cells were harvested at an OD of approximately 0.5, 0.6 and 0.7, pellet was washed once with 10% cold glycerol at 4° C. and re-suspended in 2 ml 10% glycerol (4° C.).

100 μl of competent cells were the mixed with 2-8 pg DNA and kept on ice for 2 min in an electroporation cuvette with a width of 0.2 cm. Electroporation under the following conditions: 400 0; 25 ρF; 2.5 kV (Gene Pulser, Bio-Rad). 1 ml of chilled BHI was added immediately after electroporation and incubation was performed for approximately 2 h at 37° C.

Cells were plated on BHI with 5 mg/L chloramphenicol and incubated for 2-5 d at 37° C. until the colonies of the transformants were visible. Clones were isolated and restreaked onto BHI with 5 mg/l chloramphenicol until purity of clones was obtained.

### Example 2

### Generation of Deletion Constructs

Mutation/deletion plasmids were constructed based on the vector pSacB (SEQ ID NO: 13). FIG. 1 shows a schematic map of plasmid pSacB. 5′- and 3′-flanking regions (approx. 1500 bp each) of the chromosomal fragment, which should be deleted were amplified by PCR from chromosomal DNA of Basfia succiniciproducens and introduced into said vector using standard techniques. Normally, at least 80% of the ORF were targeted for a deletion. In such a way the deletion plasmids for the lactate dehydrogenase ldhA, pSacB_delta_ldhA (SEQ ID NO: 15), the pyruvate formate lyase activating enzyme pflD pSacB_delta_pflD (SEQ ID No: 16), the pyruvate formate lyase activating enzyme pflA, pSacB_delta_pflA (SEQ ID No: 17) and the phosphoenolpyruvate craboxylase pSacB_delta_pckA (SEQ ID No: 18) were constructed. FIGS. 3, 4, 5 and 6 show schematic maps of plasmid pSacB_delta_ldhA, pSacB_delta_pflD, pSacB_delta_pflA, and pSacB_delta_pckA, respectively. The plasmid pSacB_alaD (SEQ ID NO:14) was constructed containing the 5′- and 3′-flanking regions of the pflD gene of Basfia succiniciproducens which bordered the alaD gene of Geobacillus stearothermophilus XL65-6. The alaD gene was ordered from DNA2.0. The plasmid pSacB_alaD can be used for introducing alaD gene in the pflD gene locus of Basfia succiniciproducens. FIG. 2 depicts a schematic map of plasmid pSacB_alaD (SEQ ID NO:14).

In the plasmid sequence of pSacB (SEQ ID NO:13) the sacB-gene is contained from bases 2380-3801. The sacB-promotor is contained from bases 3802-4264. The chloramphenicol gene is contained from base 526-984. The origin of replication for E. coli (on EC) is contained from base 1477-2337 (see FIG. 1).

In the plasmid sequence of pSacB_alaD (SEQ ID NO: 14) the 5′ flanking region of the pflD gene, which is homologous to the genome of Basfia succiniciproducens, is contained from bases 4-1574, while the 3′ flanking region of the pflD gene, which is homologous to the genome of Basfia succiniciproducens, is contained from bases 2694-4194. The alaD gene is contained from bases 1575-2693. The sacB gene is contained from bases 6466-7887. The sacB promoter is contained from bases 7888-8350. The chloramphenicol gene is contained from base 4612-5070. The origin of replication for E. coli (ori EC) is contained from base 5563-6423 (cf. FIG. 2).

In the plasmid sequence of pSacB_delta_idhA (SEQ ID NO: 15) the 5′ flanking region of the idhA-gene, which is homologous to the genome of Basfia succiniciproducens, is contained from bases 1519-2850, while the 3′ flanking region of the idhA-gene, which is homologous to the genome of Basfia succiniciproducens, is contained from bases 62-1518. The sacB-gene is contained from bases 5169-6590. The sacB-promoter is contained from bases 6591-7053. The chloramphenicol gene is contained from base 3315-3773. The origin of replication for E. coli (on EC) is contained from base 4266-5126 (see FIG. 3).

In the plasmid sequence of pSacB_delta_pflD (SEQ ID NO:16) the 5′ flanking region of the pflD gene, which is homologous to the genome of Basfia succiniciproducens, is contained from bases 1533-2955, while the 3′ flanking region of the pflD gene, which is homologous to the genome of Basfia succiniciproducens, is contained from bases 62-1532. The sacB gene is contained from bases 5256-6677. The sacB promoter is contained from bases 6678-7140. The chloramphenicol gene is contained from base 3402-3860. The origin of replication for E. coli (on EC) is contained from base 4353-5213 (see FIG. 4).

In the plasmid sequence of pSacB_delta_pflA (SEQ ID NO:17) the 5′ flanking region of the pflA-gene, which is homologous to the genome of Basfia succiniciproducens, is contained from bases 1506-3005, while the 3′ flanking region of the pflA-gene, which is homologous to the genome of Basfia succiniciproducens, is contained from bases 6-1505. The sacB-gene is contained from bases 5278-6699. The sacB-promoter is contained from bases 6700-7162. The chloramphenicol gene is contained from base 3424-3882. The origin of replication for E. coli (on EC) is contained from base 4375-5235 (see FIG. 5).

In the plasmid sequence of pSacB_delta_pckA (SEQ ID NO:18) the 5′ flanking region of the pckA gene, which is homologous to the genome of Basfia succiniciproducens, is contained from bases 5281-6780, while the 3′ flanking region of the pckA gene, which is homologous to the genome of Basfia succiniciproducens, is contained from bases 3766-5265. The sacB gene is contained from bases 1855-3276. The sacB promoter is contained from bases 3277-3739. The chloramphenicol gene is contained from base 1-459. The origin of replication for E. coli (on EC) is contained from base 952-1812 (see FIG. 6).

### Example 3

### Generation of Improved Succinate Alanine Strains

a) Basfia succiniciproducens DD1 was transformed as described above with the pSacB_delta_ldhA and “Campbelled in” to yield a “Campbell in” strain. Transformation and integration into the genome of Basfia succiniciproducens was confirmed by PCR yielding bands for the integrational event of the plasmid into the genome of Basfia succiniciproducens.
   The “Campbell in” strain was then “Campbelled out” using agar plates containing sucrose as a counter selection medium, selecting for the loss (of function) of the sacB gene. Therefore, the “Campbell in” strains were incubated in 25-35 ml of non selective medium (BHI containing no antibiotic) at 37° C., 220 rpm over night. The overnight culture was then streaked onto freshly prepared BHI containing sucrose plates (10%, no antibiotics) and incubated overnight at 37° C. (“first sucrose transfer”). Single colony obtained from first transfer were again streaked onto freshly prepared BHI containing sucrose plates (10%) and incubated overnight at 37° C. (“second sucrose transfer”). This procedure was repeated until a minimal completion of five transfers (“third, forth, fifth sucrose transfer”) in sucrose. The term “first to fifth sucrose transfer” refers to the transfer of a strain after chromosomal integration of a vector containing a sacB-levan-sucrase gene onto sucrose and growth medium containing agar plates for the purpose of selecting for strains with the loss of the sacB gene and the surrounding plasmid sequences. Single colony from the fifth transfer plates were inoculated onto 25-35 ml of non selective medium (BHI containing no antibiotic) and incubated at 37° C., 220 rpm over night. The overnight culture was serially diluted and plated onto BHI plates to obtain isolated single colonies.
   The “Campbelled out” strains containing either the wildtype situation of the ldhA-locus or the mutation/deletion of the ldhA-gene were confirmed by chloramphenicol sensitivity. The mutation/deletion mutants among these strains were identified and confirmed by PCR analysis. This led to the ldhA-deletion mutant Basfia succiniciproducens DD1 ΔldhA.
b) Basfia succiniciproducens DD1 ΔldhA was transformed with pSacB_delta_pflD as described above and “Campbelled in” to yield a “Campbell in” strain. Transformation and integration was confirmed by PCR. The “Campbell in” strain was then “Campbelled out” as described previously. The deletion mutants among these strains were identified and confirmed by PCR analysis. This led to the ldhA pflD-double deletion mutant Basfia succiniciproducens DD1 ΔldhA ΔpflD.
c) Basfia succiniciproducens DD1 ΔldhA ΔpflD (DD3) was transformed with pSacB_alaD as described above and “Campbelled in” to yield a “Campbell in” strain. Transformation and integration was confirmed by PCR. The “Campbell in” strain was then “Campbelled out” as described previously. The mutants among these strains were identified and confirmed by PCR analysis. This led to the ldhA pflD alaD mutant Basfia succiniciproducens DD1 ΔldhA ΔpflD alaD (DD3 alaD).
d) Basfia succiniciproducens DD1 ΔldhA ΔpflD alaD (DD3 alaD) was transformed with pSacB_delta_pckA as described above and “Campbelled in” to yield a “Campbell in” strain. Transformation and integration was confirmed by PCR. The “Campbell in” strain was then “Campbelled out” as described previously. The deletion mutants among these strains were identified and confirmed by PCR analysis. This led to the mutant Basfia succiniciproducens DD1 ΔldhA ΔpflD ΔpckA alaD (DD3 ΔpckA alaD).

### Example 4

### Cultivation of Various DD1-Strains on Glucose

### 1. Medium Preparation

The composition and preparation of the cultivation medium is as described in the following tables 2 and 3.

TABLE 2a)
Medium B4_AE (aerobic growth) composition
(pre-culture) for cultivation on glucose.

| | | Concentration |
|---|---|---|
| | Compound | [g/L] |
| 1 | Calcium carbonate | 50 |
| 2 | Succinic acid | 2.5 |
| 3 | D-(+)-Glucose | 50 |
| 4 | Salt solution* | 2, 5 |
| 5 | Sodium carbonate | 2 |
| 6 | Yeast extract | 12.5 |
| 7 | H₂O | ad 50 mL |

TABLE 2 b)
Medium B4_AN (anaerobic growth) composition
(pre-culture) for cultivation on glucose.

| | | Concentration |
|---|---|---|
| | Compound | [g/L] |
| 1 | Magnesium sulfate | 50 |
| 2 | Succinic acid | 2.5 |
| 3 | D-(+)-Glucose | 50 |
| 4 | Salt solution* | 2, 5 |
| 5 | Sodium carbonate | 2 |
| 6 | Yeast extract | 12.5 |
| 7 | H₂O | ad 50 mL |

| | Concentration |
|---|---|
| Compound | [g/L] |
| (NH₄)₂SO₄ | 150 |
| KH₂PO₄ | 100 |

*Salt solution:

TABLE 3a)
Medium B5_AE (aerobic growth) composition
(main-culture) for cultivation on glucose.

| | | Concentration |
|---|---|---|
| | Compound | [g/L] |
| 1 | Calcium carbonate | 50 |
| 2 | Succinic acid | 2.5 |
| 3 | D-(+)-Glucose | 50 |
| 4 | Salt solution* | 2, 5 |
| 5 | Ammonium sulfate | a) 6.5 |
| | | b) 10.1 |
| | | c) 13.7 |
| 6 | Sodium carbonate | 2 |
| 7 | Yeast extract | 12.5 |
| 8 | H₂O | ad 50 mL |

TABLE 3b)
Medium B5_AE (anaerobic growth) composition
(main-culture) for cultivation on glucose.

| | | Concentration |
|---|---|---|
| | Compound | [g/L] |
| 1 | Magnesium sulfate | 50 |
| 2 | Succinic acid | 2.5 |
| 3 | D-(+)-Glucose | 50 |
| 4 | Salt solution* | 2, 5 |
| 5 | Ammonium sulfate | a) 6.5 |
| | | b) 10.1 |
| | | c) 13.7 |
| 6 | Sodium carbonate | 2 |
| 7 | Yeast extract | 12.5 |
| 8 | H₂O | ad 50 mL |

| | Concentration |
|---|---|
| Compound | [g/L] |
| (NH₄)₂SO₄ | 150 |
| KH₂PO₄ | 100 |

*Salt solution:

### 2. Cultivations and Analytics

For growing the pre-culture bacteria from a freshly grown BHI-agar plate were used to inoculate a 250 ml shaking flask containing 50 ml of the liquid medium B4_AE as described in table 2a) or a 100 ml serum flask containing 50 ml of the liquid medium B4_AN described in table 2b). The flasks were incubated at 37° C. and 170 rpm (shaking diameter: 2.5 cm). Consumption of the C-sources and production of carboxylic acids was quantified via HPLC (HPLC methods are described in Table 10 and 11) after the times specified in the tables.
Cell growth was traced by measuring the absorbance at 600 nm (OD600) using a spectrophotometer (Ultrospec3000, Amersham Biosciences, Uppsala Sweden).
For growing the main culture the pre-culture was used to inoculate a 250 ml-shaking flask containing 50 ml of the liquid medium B5_AE described in table 3a) or a 100 ml-shaking flask containing 50 ml of the liquid medium B5_AN described in table 3b The flasks were incubated at 37° C. and 170 rpm (shaking diameter: 2.5 cm). Consumption of the C-sources and production of carboxylic acids was quantified via HPLC (HPLC methods are described in Table 10 and 11) after the times specified in the tables. Main cultures growing under aerobic conditions were inoculated with pre-cultures growing also under aerobic conditions. Main cultures growing under anaerobic conditions were inoculated with pre-cultures growing also under anaerobic conditions.
Cell growth was measured by measuring the absorbance at 600 nm (OD600) using a spectrophotometer (Ultrospec3000, Amersham Biosciences, Uppsala Sweden).

### 3. Results

Surprisingly the wild type strain Basfia succiniciproducens DD3 did not show any growth or alanine production under the used aerobic cultivation conditions in media B4_AE (Table 9). Accordingly, no main culture for Basfia succiniciproducens DD3 was cultivated.

The strain Basfia succiniciproducens DD3 alaD in contrast to the wild type strain Basfia succiniciproducens DD3 showed increased production of alanine under aerobic (media B4_AE and B5_AE; Table 4 and Table 5) and also anaerobic (media B4_AN and B5_AN; Table 6, Table 7, Table 8 and Table 9) cultivation conditions.

TABLE 4
Aerobic cultivation of pre-cultures of the DD3 strain, and the DD3 alaD strain.
Surprisingly the wild type strain Basfia succiniciproducens DD3 did not show
growth under the used aerobic cultivation conditions in media B4 AE.

| | | | DD3 | DD3 |
|---|---|---|---|---|
| | DD3 | DD3 | alaD | alaD |
| | pre-culture | | pre-culture | |
| Medium | Medium B4 AE | | Medium B4 AE | |
| Cultivation time [h] | 0 | 10 | 0 | 10 |
| Substrate | glucose | glucose | glucose | glucose |
| Glucose [g/L]^{a} | 0 | 0 | 0 | 18.5 |
| OD | 0.4 | 1.9 | 0.4 | 19.0 |
| Alanine [g/L]^{b} | 0.9 | 0.7 | 0.7 | 2.6 |
| Succinic acid [g/L]^{b} | 2.7 | 2.7 | 2.7 | 11.7 |
| Lactic acid [g/L]^{b} | 0.0 | 0.0 | 0.0 | 0.1 |
| Acetic acid [g/L]^{b} | 0.1 | 0.5 | 0.0 | 3.0 |
| Formic acid [g/L]^{b} | 0.0 | 0.0 | 0.0 | 0.0 |
| Pyruvic acid [g/L]^{b} | 0.0 | 1.7 | 0.0 | 3.6 |

^{a}consumption of substrate (glucose)
^{b}measured concentration of alanine, succinic acid, lactic acid, formic acid, acetic acid, pyruvic acid

TABLE 5
Aerobic cultivation of the DD3 strain, and the DD3 alaD strain

| | DD3 alaD | DD3 alaD | DD3 alaD | DD3 alaD |
|---|---|---|---|---|
| Medium | Medium B5_AE1 | | Medium B5_AE2 | |
| Incubation time [h] | 0 | 26 | 0 | 26 |
| Substrate | glucose | glucose | glucose | glucose |
| Glucose [g/L]^{a} | 0 | 47.4 | 0.0 | 27.5 |
| OD | 1.1 | 29.5 | 1.1 | 15.8 |
| Alanine [g/L]^{b} | 0.8 | 10.1 | 0.7 | 13.1 |
| Succinic acid [g/L]^{b} | 3.4 | 26.7 | 3.4 | 11.7 |
| Lactic acid [g/L]^{b} | 0.0 | 0.4 | 0.0 | 0.2 |
| Acetic acid [g/L]^{b} | 0.2 | 11.4 | 0.2 | 4.3 |
| Fumaric acid [g/L]^{b} | 0.0 | 0.0 | 0.0 | 0.0 |
| Pyruvic acid [g/L]^{b} | 0.0 | 1.0 | 0.0 | 2.0 |

¹overall concentration of (NH₄)₂SO₄:6.5 g/L
²overall concentration of (NH₄)₂SO₄:10.1 g/L
^{a}consumption of substrate (glucose)
^{b}measured concentration of alanine, succinic acid, lactic acid, formic acid, acetic acid, pyruvic acid

TABLE 6
Anaerobic cultivation of pre-cultures of the DD3 strain, and
the DD3 alaD strain (Medium B4_AN).

| | | | DD3 | DD3 |
|---|---|---|---|---|
| | DD3 | DD3 | alaD | alaD |
| | pre-culture | | pre-culture | |
| Medium | Medium B4_AN | | Medium B4 AN | |
| Incubation time [h] | 0 | 10 | 0 | 10 |
| substrate | glucose | glucose | glucose | glucose |
| Glucose [g/L]^{a} | 0 | 44.2 | 0 | 30.8 |
| OD | 0.4 | 27.0 | 0.4 | 20.5 |
| Alanine [g/L]^{b} | 0.7 | 0.7 | 0.7 | 2.9 |
| Succinic acid [g/L]^{b} | 2.8 | 33.9 | 2.8 | 25.8 |
| Lactic acid [g/L]^{b} | 0.0 | 0.2 | 0.0 | 0.2 |
| Acetic acid [g/L]^{b} | 0.1 | 1.2 | 0.1 | 2.6 |
| Fumaric acid [g/L]^{b} | 0.0 | 0.0 | 0.0 | 0.0 |
| Pyruvic acid [g/L]^{b} | 0.0 | 2.9 | 0.0 | 1.4 |

^{a}consumption of substrate (glucose)
^{b}measured concentration of alanine, succinic acid, lactic acid, formic acid, acetic acid, pyruvic acid

TABLE 7
Anaerobic cultivation of the DD3 strain, and
the DD3 alaD strain (Medium B5_AN).

| | | | DD3 | DD3 |
|---|---|---|---|---|
| | DD3 | DD3 | alaD | alaD |
| Medium | Medium B5_AN1 | | | |
| Incubation time [h] | 0 | 24 | 0 | 24 |
| substrate | glucose | glucose | glucose | glucose |
| Glucose [g/L]^{a} | 0 | 14.8 | 0 | 6.8 |
| OD | 1.5 | 7.0 | 1.1 | 2.4 |
| Alanine [g/L]^{b} | 0.7 | 0.8 | 0.9 | 3.1 |
| Succinic acid [g/L]^{b} | 4.2 | 15.1 | 4.1 | 8.2 |
| Lactic acid [g/L]^{b} | 0.0 | 0.2 | 0.0 | 0.1 |
| Acetic acid [g/L]^{b} | 0.2 | 1.3 | 0.2 | 0.7 |
| Fumaric acid [g/L]^{b} | 0.0 | 0.0 | 0.0 | 0.0 |
| Pyruvic acid [g/L]^{b} | 0.0 | 1.4 | 0.0 | 0.0 |

¹overall concentration of (NH₄)₂SO₄:6.5 g/L
^{a}consumption of substrate (glucose)
^{b}measured concentration of alanine, succinic acid, lactic acid, formic acid, acetic acid, pyruvic acid

TABLE 8
Anaerobic cultivation of the DD3 strain, and
the DD3 alaD strain (Medium B5_AN)

| | | | DD3 | DD3 |
|---|---|---|---|---|
| | DD3 | DD3 | alaD | alaD |
| Medium | Medium B5_AN1 | | | |
| Incubation time [h] | 0 | 24 | 0 | 24 |
| substrate | glucose | glucose | glucose | glucose |
| Glucose [g/L]^{a} | 0 | 5.4 | 0 | 5.7 |
| OD | 1.2 | 2.5 | 2.2 | 1.6 |
| Alanine [g/L]^{b} | 0.6 | 0.9 | 0.9 | 2.9 |
| Succinic acid [g/L]^{b} | 4.1 | 8.3 | 4.2 | 7.6 |
| Lactic acid [g/L]^{b} | 0.0 | 0.2 | 0.0 | 0.1 |
| Acetic acid [g/L]^{b} | 0.1 | 0.6 | 0.2 | 0.5 |
| Fumaric acid [g/L]^{b} | 0.0 | 0.0 | 0.0 | 0.0 |
| Pyruvic acid [g/L]^{b} | 0.0 | 0.6 | 0.0 | 0.0 |

¹overall concentration of (NH₄)₂SO₄:10.1 g/L
^{a}consumption of substrate (glucose)
^{b}measured concentration of alanine, succinic acid, lactic acid, formic acid, acetic acid, pyruvic acid

TABLE 9
Anaerobic cultivation of the DD3 strain, and
the DD3 alaD strain (Medium B5_AN).

| | | | DD3 | DD3 |
|---|---|---|---|---|
| | DD3 | DD3 | alaD | alaD |
| Medium | Medium B5 AN1 | | | |
| Incubation time [h] | 0 | 24 | 0 | 24 |
| substrate | glucose | glucose | glucose | glucose |
| Glucose [g/L]^{a} | 0 | 3.7 | 0 | 4.1 |
| OD | 1.1 | 1.9 | 1.0 | 4.0 |
| Alanine [g/L]^{b} | 0.9 | 0.9 | 0.8 | 2.9 |
| Succinic acid [g/L]^{b} | 4.1 | 6.6 | 4.1 | 6.9 |
| Lactic acid [g/L]^{b} | 0.0 | 0.1 | 0.0 | 0.1 |
| Acetic acid [g/L]^{b} | 0.2 | 0.5 | 0.2 | 0.4 |
| Fumaric acid [g/L]^{b} | 0.0 | 0.0 | 0.0 | 0.0 |
| Pyruvic acid [g/L]^{b} | 0.0 | 0.5 | 0.0 | 0.0 |

¹overall concentration of (NH₄)₂SO₄:13.7 g/L
^{a}consumption of substrate (glucose)
^{b}measured concentration of alanine, succinic acid, lactic acid, formic acid, acetic acid, pyruvic acid

TABLE 10
HPLC method (ZX-THF50) for analysis of glucose, succinic acid, formic
acid, lactic acid, acetic acid, pyruvic acid, propionic acid and ethanol.

| HPLC column | Aminex HPX-87 H, 300*7.8 mm (BioRad) | | | |
|---|---|---|---|---|
| Precolumn | Cation H | | | |
| Temperature | 50° C. | | | |
| Eluent flow rate | 0.50 ml/min | | | |
| Injection volume | 5.0 μl | | | |
| Diode array detector | RI-Detector | | | |
| Runtime | 28 min | | | |
| max. pressure | 140 bar | | | |
| Eluent A | 5 mM H₂SO₄ | | | |
| Eluent B | 5 mM H₂SO₄ | | | |
| Gradient | Time [min] | A[%] | B[%] | Flow [ml/min] |
| | 0.0 | 50 | 50 | 0.50 |
| | 28.0 | 50 | 50 | 0.50 |

TABLE 11
HPLC method AA-Alanin for analysis of alanine.

| HPLC column | Gemini C18, 150*4, 6 mm (Phenomenex) | | | |
|---|---|---|---|---|
| Precolumn | C18 Gemini | | | |
| Temperature | 40° C. | | | |
| Eluent flow rate | 1.50 ml/min | | | |
| Injection volume | 0.5 ml | | | |
| Diode array detector | UV-Detector | | | |
| Runtime | 12 min | | | |
| max. pressure | 300 bar | | | |
| Eluent A | 40 mM NaH₂PO₄ × H₂O | | | |
| | (pH 7, 8, 1, 85 ml/l NaOH [50%]) | | | |
| Eluent B | Acetonitril:Methanol:Water 45:45:10 | | | |
| Gradient | Time [min] | A[%] | B[%] | Flow [ml/min] |
| | 0 | 80 | 20 | 1.5 |
| | 6 | 80 | 20 | 1.5 |
| | 7 | 0 | 100 | 1.5 |
| | 11.5 | 0 | 100 | 1.5 |
| | 12.5 | 80 | 20 | 1.5 |

### Example 5

### Measurement of Activity of Alanine Dehydrogenase (alaD)

Enzyme activity assay Enzyme activities were measured spectrophotometrically at 33° C. Cells before starting alanine production were harvested by centrifugation (5,000×g, 4° C.; 10 min). The cell pellet was washed once with extraction buffer (100 mM Tris-HCl, pH 7.5, 20 mM KCl, 20 mM MgCl2, 0.1 mM EDTA, 2 mM DTT). The resulting cell suspensions were sonicated using an ultrasonic homogenizer in an ice-water bath for 15 min. Cell debris was removed by centrifugation (10,000×g, 4° C.; 30 min). The cell lysates, thus, produced were subsequently used as crude extracts for enzyme assays. Protein concentrations were measured using a protein assay kit (Bio-Rad, USA). AlaDH catalyzes formation of alanine from pyruvate and ammonium ion with consuming NADH. AlaDH activity was measured by following the decrease in absorbance of NADH at 340 nm, using a spectrophotometer. An assay mixture contained 0.5 mM NADH, 2 mM pyruvate, 100 mM NH4Cl in 100 mM Tris-HCl, pH 8.5. The reaction was started by the addition of the crude extracts to the assay mixture (Jojima et al. (2010): Engineering of sugar metabolism of Corynebacterium glutamicum for production of amino acid L-alanine under oxygen deprivation, Appl. Microbiol. 87, 159-165.

SEQUENCES

SEQ ID NO: 1 (nucleotide sequence of 16 S rDNA of strain DD1)
(Basfia succiniciproducens)
tttgatcctggctcagattgaacgctggcggcaggcttaacacatgcaagtcgaacggtagcgggaggaa
agcttgctttctttgccgacgagtggcggacgggtgagtaatgcttggggatctggcttatggaggggga
taacgacgggaaactgtcgctaataccgcgtaatatcttcggattaaagggtgggactttcgggccaccc
gccataagatgagcccaagtgggattaggtagttggtggggtaaaggcctaccaagccgacgatctctag
ctggtctgagaggatgaccagccacactggaactgagacacggtccagactcctacgggaggcagcagtg
gggaatattgcacaatggggggaaccctgatgcagccatgccgcgtgaatgaagaaggccttcgggttgt
aaagttctttcggtgacgaggaaggtgtttgttttaataggacaagcaattgacgttaatcacagaagaa
gcaccggctaactccgtgccagcagccgcggtaatacggagggtgcgagcgttaatcggaataactgggc
gtaaagggcatgcaggcggacttttaagtgagatgtgaaagccccgggcttaacctgggaattgcatttc
agactgggagtctagagtactttagggaggggtagaattccacgtgtagcggtgaaatgcgtagagatgt
ggaggaataccgaaggcgaaggcagccccttgggaagatactgacgctcatatgcgaaagcgtggggagc
aaacaggattagataccctggtagtccacgcggtaaacgctgtcgatttggggattgggctttaggcctg
gtgctcgtagctaacgtgataaatcgaccgcctggggagtacggccgcaaggttaaaactcaaatgaatt
gacgggggcccgcacaagcggtggagcatgtggtttaattcgatgcaacgcgaagaaccttacctactct
tgacatccagagaatcctgtagagatacgggagtgccttcgggagctctgagacaggtgctgcatggctg
tcgtcagctcgtgttgtgaaatgttgggttaagtcccgcaacgagcgcaacccttatcctttgttgccag
catgtaaagatgggaactcaaaggagactgccggtgacaaaccggaggaaggtggggatgacgtcaagtc
atcatggcccttacgagtagggctacacacgtgctacaatggtgcatacagagggcggcgataccgcgag
gtagagcgaatctcagaaagtgcatcgtagtccggattggagtctgcaactcgactccatgaagtcggaa
tcgctagtaatcgcaaatcagaatgttgcggtgaatacgttcccgggccttgtacacaccgcccgtcaca
ccatgggagtgggttgtaccagaagtagatagcttaaccttcggggggggcgtttaccacggtatgattc
atgactggggtgaagtcgtaacaaggtaaccgtaggggaacctgcgg
SEQ ID NO: 2 (nucleotide sequence of 23 S rDNA of strain DD1)
(Basfia succiniciproducens)
agtaataacg aacgacacag gtataagaat acttgaggtt gtatggttaa gtgactaagc
gtacaaggtg gatgccttgg caatcagagg cgaagaagga cgtgctaatc tgcgaaaagc
ttgggtgagt tgataagaag cgtctaaccc aagatatccg aatggggcaa cccagtagat
gaagaatcta ctatcaataa ccgaatccat aggttattga ggcaaaccgg gagaactgaa
acatctaagt accccgagga aaagaaatca accgagatta cgtcagtagc ggcgagcgaa
agcgtaagag ccggcaagtg atagcatgag gattagagga atcggctggg aagccgggcg
gcacagggtg atagccccgt acttgaaaat cattgtgtgg tactgagctt gcgagaagta
gggcgggaca cgagaaatcc tgtttgaaga aggggggacc atcctccaag gctaaatact
cctgattgac cgatagtgaa ccagtactgt gaaggaaagg cgaaaagaac cccggtgagg
ggagtgaaat agaacctgaa accttgtacg tacaagcagt gggagcccgc gagggtgact
gcgtaccttt tgtataatgg gtcagcgact tatattatgt agcgaggtta accgaatagg
ggagccgaag ggaaaccgag tcttaactgg gcgtcgagtt gcatgatata gacccgaaac
ccggtgatct agccatgggc aggttgaagg ttgggtaaca ctaactggag gaccgaaccg
actaatgttg aaaaattagc ggatgacctg tggctggggg tgaaaggcca atcaaaccgg
gagatagctg gttctccccg aaatctattt aggtagagcc ttatgtgaat accttcgggg
gtagagcact gtttcggcta gggggccatc ccggcttacc aacccgatgc aaactgcgaa
taccgaagag taatgcatag gagacacacg gcgggtgcta acgttcgtcg tggagaggga
aacaacccag accgccagct aaggtcccaa agtttatatt aagtgggaaa cgaagtggga
aggcttagac agctaggatg ttggcttaga agcagccatc atttaaagaa agcgtaatag
ctcactagtc gagtcggcct gcgcggaaga tgtaacgggg ctcaaatata gcaccgaagc
tgcggcatca ggcgtaagcc tgttgggtag gggagcgtcg tgtaagcgga agaaggtggt
tcgagagggc tgctggacgt atcacgagtg cgaatgctga cataagtaac gataaaacgg
gtgaaaaacc cgttcgccgg aagaccaagg gttcctgtcc aacgttaatc ggggcagggt
gagtcggccc ctaaggcgag gctgaagagc gtagtcgatg ggaaacgggt taatattccc
gtacttgtta taattgcgat gtggggacgg agtaggttag gttatcgacc tgttggaaaa
ggtcgtttaa gttggtaggt ggagcgttta ggcaaatccg gacgcttatc aacaccgaga
gatgatgacg aggcgctaag gtgccgaagt aaccgatacc acacttccag gaaaagccac
taagcgtcag attataataa accgtactat aaaccgacac aggtggtcag gtagagaata
ctcaggcgct tgagagaact cgggtgaagg aactaggcaa aatagcaccg taacttcggg
agaaggtgcg ccggcgtaga ttgtagaggt atacccttga aggttgaacc ggtcgaagtg
acccgctggc tgcaactgtt tattaaaaac acagcactct gcaaacacga aagtggacgt
atagggtgtg atgcctgccc ggtgctggaa ggttaattga tggcgttatc gcaagagaag
cgcctgatcg aagccccagt aaacggcggc cgtaactata acggtcctaa ggtagcgaaa
ttecttgtcg ggtaagttcc gacctgcacg aatggcataa tgatggccag gctgtctcca
cccgagactc agtgaaattg aaatcgccgt gaagatgcgg tgtacccgcg gctagacgga
aagaccccgt gaacctttac tatagcttga cactgaacct tgaattttga tgtgtaggat
aggtgggagg ctttgaagcg gtaacgccag ttatcgtgga gccatccttg aaataccacc
ctttaacgtt tgatgttcta acgaagtgcc cggaacgggt actcggacag tgtctggtgg
gtagtttgac tggggcggtc tcctcccaaa gagtaacgga ggagcacgaa ggtttgctaa
tgacggtcgg acatcgtcag gttagtgcaa tggtataagc aagcttaact gcgagacgga
caagtcgagc aggtgcgaaa gcaggtcata gtgatccggt ggttctgaat ggaagggcca
tcgctcaacg gataaaaggt actccgggga taacaggctg ataccgccca agagttcata
tcgacggcgg tgtttggcac ctcgatgtcg gctcatcaca tcctggggct gaagtaggtc
ccaagggtat ggctgttcgc catttaaagt ggtacgcgag ctgggtttaa aacgtcgtga
gacagtttgg tccctatctg ccgtgggcgt tggagaattg agaggggctg ctcctagtac
gagaggaccg gagtggacgc atcactggtg ttccggttgt gtcgccagac gcattgccgg
gtagctacat gcggaagaga taagtgctga aagcatctaa gcacgaaact tgcctcgaga
tgagttctcc cagtatttaa tactgtaagg gttgttggag acgacgacgt agataggccg
ggtgtgtaag cgttgcgaga cgttgagcta accggtacta attgcccgag aggcttagcc
atacaacgct caagtgtttt tggtagtgaa agttattacg gaataagtaa gtagtcaggg
aatcggct
SEQ ID NO: 3 (nucleotide sequence of alaD-gene)
(Geobacillus stearothermophilus optimized for E. Coli)
atgaaaattggcatccctaaagagattaagaacaatgaaaaccgtgtagcaatcaccccggcaggtgtta
tgactctggttaaagcgggccacgatgtgtacgtcgaaaccgaagcgggtgccggcagcggcttcagcga
cagcgagtatgagaaggcgggtgcggttattgtgactaaggcggaggacgcttgggcagccgaaatggtt
ctgaaggtgaaagaaccgctggcggaggagtttcgctattttcgtccgggtctgattttgttcacctacc
tgcacctggctgcggccgaggcgctgaccaaggcactggtggagcagaaggttgttggcatcgcgtacga
aacggttcaactggcgaatggttccctgccgctgctgacccctatgtctgaagttgcgggtcgcatgagc
gttcaagtcggcgctcagtttctggagaaaccgcacggtggcaagggcattttgctgggtggtgttccgg
gtgtccgccgtggtaaagtgacgatcattggcggtggtacggccggtacgaacgcggccaagattgccgt
aggtctgggtgcagatgtgaccattctggacatcaacgcggaacgtttgcgtgagctggacgatctgttt
ggcgaccaagtcaccaccctgatgagcaacagctaccacatcgcggagtgcgtccgtgaaagcgatttgg
tcgttggtgcggtgctgatcccgggtgcaaaagccccgaaactggtgaccgaggagatggtccgtagcat
gaccccgggttcggttctggtcgacgtggcaattgaccagggcggtatcttcgaaaccaccgaccgcgtc
acgacccatgatgacccgacctatgtgaaacatggcgtggttcactatgcggtcgcgaatatgccgggtg
cagtgccgcgcacgtccacgttcgcgctgacgaacgtgacgattccatacgctctgcagatcgccaataa
gggctatcgtgcggcgtgtctggataatccggcattgctgaaaggcatcaataccctggatggtcatatc
gtttacgaggctgtggctgcagcacacaacatgccgtacactgatgtccatagcttgctgcaaggctaa
SEQ ID NO: 4 (amino acid sequence of the enzyme encoded by the above AlaD-gene)
(Geobacillus stearothermophilus)
mkigipkeiknnenrvaitpagvmtlvkaghdvyveteagagsgfsdseyekagavivtkaedawaaemv
lkvkeplaeefryfrpglilftylhlaaaealtkalveqkvvgiayetvqlangslplltpmsevagrms
vqvgaqflekphggkgillggvpgvrrgkvtiigggtagtnaakiavglgadvtildinaerlrelddlf
gdqvttlmsnsyhiaecvresdlvvgavlipgakapklvteemvrsmtpgsvlvdvaidqggifettdrv
tthddptyvkhgvvhyavanmpgavprtstfaltnvtipyalqiankgyraacldnpallkgintldghi
vyeavaaahnmpytdvhsllqg
SEQ ID NO: 5 (nucleotide sequence of IdhA gene)
(Basfia succiniciproducens)
ttgacaaaatcagtatgtttaaataaggagctaactatgaaagttgccgtttacagtactaaaaattatg
atcgcaaacatctggatttggcgaataaaaaatttaattttgagcttcatttctttgattttttacttga
tgaacaaaccgcgaaaatggcggagggcgccgatgccgtctgtattttcgtcaatgatgatgcgagccgc
ccggtgttaacaaagttggcgcaaatcggagtgaaaattatcgctttacgttgtgccggttttaataatg
tggatttggaggcggcaaaagagctgggattaaaagtcgtacgggtgcctgcgtattcgccggaagccgt
tgccgagcatgcgatcggattaatgctgactttaaaccgccgtatccataaggcttatcagcgtacccgc
gatgcgaatttttctctggaaggattggtcggttttaatatgttcggcaaaaccgccggagtgattggta
cgggaaaaatcggcttggcggctattcgcattttaaaaggcttcggtatggacgttctggcgtttgatcc
ttttaaaaatccggcggcggaagcgttgggcgcaaaatatgtcggtttagacgagctttatgcaaaatcc
catgttatcactttgcattgcccggctacggcggataattatcatttattaaatgaagcggcttttaata
aaatgcgcgacggtgtaatgattattaataccagccgcggcgttttaattgacagccgggcggcaatcga
agcgttaaaacggcagaaaatcggcgctctcggtatggatgtttatgaaaatgaacgggatttgtttttc
gaggataaatctaacgatgttattacggatgatgtattccgtcgcctttcttcctgtcataatgtgcttt
ttaccggtcatcaggcgtttttaacggaagaagcgctgaataatatcgccgatgtgactttatcgaatat
tcaggcggtttccaaaaatgcaacgtgcgaaaatagcgttgaaggctaa
SEQ ID NO: 6 (amino acid sequence of the enzyme encoded by the above IdhA-gene)
(Basfia succiniciproducens)
MTKSVCLNKELTMKVAVYSTKNYDRKHLDLANKKFNFELHFFDFLLDEQTAKMAEGADAVCIFVNDDASR
PVLTKLAQIGVKIIALRCAGFNNVDLEAAKELGLKVVRVPAYSPEAVAEHAIGLMLTLNRRIHKAYQRTR
DANFSLEGLVGFNMEGKTAGVIGTGKIGLAAIRILKGFGMDVLAFDPFKNPAAEALGAKYVGLDELYAKS
HVITLHCPATADNYHLLNEAAFNKMRDGVMIINTSRGVLIDSRAAIEALKRQKIGALGMDVYENERDLFF
EDKSNDVITDDVFRRLSSCHNVLFTGHQAFLTEEALNNIADVTLSNIQAVSKNATCENSVEG
SEQ ID NO: 7 (nucleotide sequence of pflD-gene)
(Basfia succiniciproducens)
atggctgaattaacagaagctcaaaaaaaagcatgggaaggattcgttcccggtgaatggcaaaacggcg
taaatttacgtgactttatccaaaaaaactatactccgtatgaaggtgacgaatcattcttagctgatgc
gactcctgcaaccagcgagttgtggaacagcgtgatggaaggcatcaaaatcgaaaacaaaactcacgca
cctttagatttcgacgaacatactccgtcaactatcacttctcacaagcctggttatatcaataaagatt
tagaaaaaatcgttggtcttcaaacagacgctccgttaaaacgtgcaattatgccgtacggcggtatcaa
aatgatcaaaggttcttgcgaagtttacggtcgtaaattagatccgcaagtagaatttattttcaccgaa
tatcgtaaaacccataaccaaggcgtattcgacgtttatacgccggatattttacgctgccgtaaatcag
gcgtgttaaccggtttaccggatgcttacggtcgtggtcgtattatcggtgactaccgtcgtttagcggt
atacggtattgattacctgatgaaagataaaaaagcccaattcgattcattacaaccgcgtttggaagcg
ggcgaagacattcaggcaactatccaattacgtgaagaaattgccgaacaacaccgcgctttaggcaaaa
tcaaagaaatggcggcatcttacggttacgacatttccggccctgcgacaaacgcacaggaagcaatcca
atggacatattttgcttatctggcagcggttaaatcacaaaacggtgcggcaatgtcattcggtcgtacg
tctacattcttagatatctatatcgaacgtgacttaaaacgcggtttaatcactgaacaacaggcgcagg
aattaatggaccacttagtaatgaaattacgtatggttcgtttcttacgtacgccggaatacgatcaatt
attctcaggcgacccgatgtgggcaaccgaaactatcgccggtatgggcttagacggtcgtccgttggta
actaaaaacagcttccgcgtattacatactttatacactatgggtacttctccggaaccaaacttaacta
ttctttggtccgaacaattacctgaagcgttcaaacgtttctgtgcgaaagtatctattgatacttcctc
cgtacaatacgaaaatgatgacttaatgcgtcctgacttcaacaacgatgactatgcaatcgcatgctgc
gtatcaccgatggtcgtaggtaaacaaatgcaattcttcggtgcgcgcgcaaacttagctaaaactatgt
tatacgcaattaacggcggtatcgatgagaaaaatggtatgcaagtcggtcctaaaactgcgccgattac
agacgaagtattgaatttcgataccgtaatcgaacgtatggacagtttcatggactggttggcgactcaa
tatgtaaccgcattgaacatcatccacttcatgcacgataaatatgcatatgaagcggcattgatggcgt
tccacgatcgcgacgtattccgtacaatggcttgcggtatcgcgggtctttccgtggctgcggactcatt
atccgcaatcaaatatgcgaaagttaaaccgattcgcggcgacatcaaagataaagacggtaatgtcgtg
gcctcgaatgttgctatcgacttcgaaattgaaggcgaatatccgcaattcggtaacaatgatccgcgtg
ttgatgatttagcggtagacttagttgaacgtttcatgaaaaaagttcaaaaacacaaaacttaccgcaa
cgcaactccgacacaatctatcctgactatcacttctaacgtggtatacggtaagaaaaccggtaatact
ccggacggtcgtcgagcaggcgcgccattcggaccgggtgcaaacccaatgcacggtcgtgaccaaaaag
gtgcggttgcttcacttacttctgtggctaaacttccgttcgcttacgcgaaagacggtatttcatatac
cttctctatcgtaccgaacgcattaggtaaagatgacgaagcgcaaaaacgcaaccttgccggtttaatg
gacggttatttccatcatgaagcgacagtggaaggcggtcaacacttgaatgttaacgttcttaaccgtg
aaatgttgttagacgcgatggaaaatccggaaaaatacccgcaattaaccattcgtgtttcaggttacgc
ggttcgtttcaactcattaactaaagagcaacaacaagacgtcatcactcgtacgtttacacaatcaatg
taa
SEQ ID NO: 8 (amino acid sequence of the enzyme encoded by the above pflD-gene)
(Basfia succiniciproducens)
MAELTEAQKKAWEGFVPGEWQNGVNLRDFIQKNYTPYEGDESFLADATPATSELWNSVMEGIKIENKTHA
PLDFDEHTPSTITSHKPGYINKDLEKIVGLQTDAPLKRAIMPYGGIKMIKGSCEVYGRKLDPQVEFIFTE
YRKTHNQGVFDVYTPDILRCRKSGVLTGLPDAYGRGRIIGDYRRLAVYGIDYLMKDKKAQFDSLQPRLEA
GEDIQATIQLREEIAEQHRALGKIKEMAASYGYDISGPATNAQEAIQWTYFAYLAAVKSQNGAAMSFGRT
STFLDIYIERDLKRGLITEQQAQELMDHLVMKLRMVRFLRTPEYDQLFSGDPMWATETIAGMGLDGRPLV
TKNSFRVLHTLYTMGTSPEPNLTILWSEQLPEAFKRECAKVSIDTSSVQYENDDLMRPDFNNDDYAIACC
VSPMVVGKQMQFFGARANLAKTMLYAINGGIDEKNGMQVGPKTAPITDEVLNFDTVIERMDSFMDWLATQ
YVTALNIIHFMHDKYAYEAALMAFHDRDVFRTMACGIAGLSVAADSLSAIKYAKVKPIRGDIKDKDGNVV
ASNVAIDFEIEGEYPQFGNNDPRVDDLAVDLVERFMKKVQKHKTYRNATPTQSILTITSNVVYGKKTGNT
PDGRRAGAPFGPGANPMHGRDQKGAVASLTSVAKLPFAYAKDGISYTFSIVPNALGKDDEAQKRNLAGLM
DGYFHHEATVEGGQHLNVNVLNREMLLDAMENPEKYPQLTIRVSGYAVRFNSLTKEQQQDVITRTFTQSM
SEQ ID NO: 9 (nucleotide sequence of pflA-gene)
(Basfia succiniciproducens)
atgtcggttttaggacgaattcattcatttgaaacctgcgggacagttgacgggccgggaatccgcttta
ttttatttttacaaggctgcttaatgcgttgtaaatactgccataatagagacacctgggatttgcacgg
cggtaaagaaatttccgttgaagaattaatgaaagaagtggtgacctatcgccattttatgaacgcctcg
ggcggcggagttaccgcttccggcggtgaagctattttacaggcggaatttgtacgggactggttcagag
cctgccataaagaaggaattaatacttgcttggataccaacggtttcgtccgtcatcatgatcatattat
tgatgaattgattgatgacacggatcttgtgttgcttgacctgaaagaaatgaatgaacgggttcacgaa
agcctgattggcgtgccgaataaaagagtgctcgaattcgcaaaatatttagcggatcgaaatcagcgta
cctggatccgccatgttgtagtgccgggttatacagatagtgacgaagatttgcacatgctggggaattt
cattaaagatatgaagaatatcgaaaaagtggaattattaccttatcaccgtctaggcgcccataaatgg
gaagtactcggcgataaatacgagcttgaagatgtaaaaccgccgacaaaagaattaatggagcatgtta
aggggttgcttgcaggctacgggcttaatgtgacatattag
SEQ ID NO: 10 (amino acid sequence of the enzyme encoded by the above pflA-gene)
(Basfia succiniciproducens)
MSVLGRIHSFETCGTVDGPGIRFILFLQGCLMRCKYCHNRDTWDLHGGKEISVEELMKEVVTYRHFMNAS
GGGVTASGGEAILQAEFVRDWFRACHKEGINTCLDTNGFVRHHDHIIDELIDDTDLVLLDLKEMNERVHE
SLIGVPNKRVLEFAKYLADRNQRTWIRHVVVPGYTDSDEDLHMLGNFIKDMKNIEKVELLPYHRLGAHKW
EVLGDKYELEDVKPPTKELMEHVKGLLAGYGLNVTY
SEQ ID NO: 11 (nucleotide sequence of pckA-gene)
(Basfia succiniciproducens)
atgacagatcttaatcaattaactcaagaacttggtgctttaggtattcatgatgtacaagaagttgtgt
ataacccgagctatgaacttctttttgcggaagaaaccaaaccaggtttagacggttatgaaaaaggtac
tgtaactaatcaaggagcggttgctgtaaataccggtatttttaccggtcgttctccgaaagataaatat
atcgttttagacgacaaaactaaagataccgtatggtggaccagcgaaaaagttaaaaacgataacaaac
caatgagtcaagatacctggaacagtttgaaaggtttagttgccgatcaactttccggtaaacgtttatt
tgttgttgacgcattctgtggcgcgaataaagatacgcgtttagctgttcgtgtggttactgaagttgca
tggcaggcgcattttgtaacaaatatgtttatccgcccttcagcggaagaattaaaaggtttcaaacctg
atttcgtggtaatgaacggtgcaaaatgtacaaatcctaactggaaagagcaaggattaaattccgaaaa
cttcgttgcgttcaacattacagaaggcgttcaattaatcggcggtacttggtacggcggtgaaatgaaa
aaaggtatgttctcaatgatgaactacttcttaccacttcgcggtattgcatcaatgcactgttccgcaa
acgttggtaaagacggcgataccgcaattttcttcggtttgtcaggtacaggtaaaactacattatcaac
agatcctaaacgtcaactaatcggtgatgacgaacacggttgggacgatgaaggcgtatttaacttcgaa
ggtggttgctacgcgaaaaccattaacttatccgctgaaaacgagccggatatctatggcgctatcaaac
gtgacgcattattggaaaacgtggtcgttttagataacggtgacgttgactatgcagacggttccaaaac
agaaaatacacgtgtttcttatccgatttatcacattcaaaatatcgttaaacctgtttctaaagctggc
ccggcaactaaagttatcttcttgtctgccgatgcattcggtgtattaccgccggtgtctaaattaactc
cggaacaaaccaaatactatttcttatccggttttactgcgaaattagcgggcacagagcgtggtattac
agagcctacaccaacattttctgcatgttttggtgcggctttcttaagcttgcatccgacgcaatatgcc
gaagtgttagtaaaacgtatgcaagaatcaggtgcggaagcgtatcttgttaatacaggttggaacggta
ccggcaaacgtatctcaattaaagatacccgtggtattattgatgcaattttagacggctcaattgataa
agcggaaatgggctcattaccaatcttcgatttctcaattcctaaagcattacctggtgttaaccctgca
atcttagatccgcgcgatacttatgcggataaagcgcaatgggaagaaaaagctcaagatcttgcaggtc
gctttgtgaaaaactttgaaaaatataccggtacggcggaaggtcaggcattagttgctgccggtcctaa
agcataa
SEQ ID NO: 12 (amino acid sequence of the enzyme encoded by the above pckA-gene)
(Basfia succiniciproducens)
MTDLNQLTQELGALGIHDVQEVVYNPSYELLFAEETKPGLDGYEKGTVTNQGAVAVNTGIF
TGRSPKDKY
IVLDDKTKDTVWWTSEKVKNDNKPMSQDTWNSLKGLVADQLSGKRLFVVDAFCGANKDT
RLAVRVVTEVA
WQAHFVTNMFIRPSAEELKGFKPDFVVMNGAKCTNPNWKEQGLNSENFVAFNITEGVQLI
GGTWYGGEMK
KGMFSMMNYFLPLRGIASMHCSANVGKDGDTAIFFGLSGTGKTTLSTDPKRQLIGDDEHG
WDDEGVFNFE
GGCYAKTINLSAENEPDIYGAIKRDALLENVVVLDNGDVDYADGSKTENTRVSYPIYHIQNIV
KPVSKAG
PATKVIFLSADAFGVLPPVSKLTPEQTKYYFLSGFTAKLAGTERGITEPTPTFSACFGAAFLS
LHPTQYA
EVLVKRMQESGAEAYLVNTGWNGTGKRISIKDTRGIIDAILDGSIDKAEMGSLPIFDFSIPKA
LPGVNPA
ILDPRDTYADKAQWEEKAQDLAGRFVKNFEKYTGTAEGQALVAAGPKA
SEQ ID NO: 13 (complete nucleotide sequence of plasmid pSacB)
(artificial)
tcgagaggcctgacgtcgggcccggtaccacgcgtcatatgactagttcggacctagggatatcgtcgac
atcgatgctcttctgcgttaattaacaattgggatcctctagactccataggccgctttcctggctttgc
ttccagatgtatgctctcctccggagagtaccgtgactttattttcggcacaaatacaggggtcgatgga
taaatacggcgatagtttcctgacggatgatccgtatgtaccggcggaagacaagctgcaaacctgtcag
atggagattgatttaatggcggatgtgctgagagcaccgccccgtgaatccgcagaactgatccgctatg
tgtttgcggatgattggccggaataaataaagccgggcttaatacagattaagcccgtatagggtattat
tactgaataccaaacagcttacggaggacggaatgttacccattgagacaaccagactgccttctgatta
ttaatatttttcactattaatcagaaggaataaccatgaattttacccggattgacctgaatacctggaa
tcgcagggaacactttgccctttatcgtcagcagattaaatgcggattcagcctgaccaccaaactcgat
attaccgctttgcgtaccgcactggcggagacaggttataagttttatccgctgatgatttacctgatct
cccgggctgttaatcagtttccggagttccggatggcactgaaagacaatgaacttatttactgggacca
gtcagacccggtctttactgtctttcataaagaaaccgaaacattctctgcactgtcctgccgttatttt
ccggatctcagtgagtttatggcaggttataatgcggtaacggcagaatatcagcatgataccagattgt
ttccgcagggaaatttaccggagaatcacctgaatatatcatcattaccgtgggtgagttttgacgggat
ttaacctgaacatcaccggaaatgatgattattttgccccggtttttacgatggcaaagtttcagcagga
aggtgaccgcgtattattacctgtttctgtacaggttcatcatgcagtctgtgatggctttcatgcagca
cggtttattaatacacttcagctgatgtgtgataacatactgaaataaattaattaattctgtatttaag
ccaccgtatccggcaggaatggtggctttttttttatattttaaccgtaatctgtaatttcgtttcagac
tggttcaggatgagctcgcttggactcctgttgatagatccagtaatgacctcagaactccatctggatt
tgttcagaacgctcggttgccgccgggcgttttttattggtgagaatccaagcactagcggcgcgccggc
cggcccggtgtgaaataccgcacagatgcgtaaggagaaaataccgcatcaggcgctcttccgcttcctc
gctcactgactcgctgcgctcggtcgttcggctgcggcgagcggtatcagctcactcaaaggcggtaata
cggttatccacagaatcaggggataacgcaggaaagaacatgtgagcaaaaggccagcaaaaggccagga
accgtaaaaaggccgcgttgctggcgtttttccataggctccgcccccctgacgagcatcacaaaaatcg
acgctcaagtcagaggtggcgaaacccgacaggactataaagataccaggcgtttccccctggaagctcc
ctcgtgcgctctcctgttccgaccctgccgcttaccggatacctgtccgcctttctcccttcgggaagcg
tggcgctttctcatagctcacgctgtaggtatctcagttcggtgtaggtcgttcgctccaagctgggctg
tgtgcacgaaccccccgttcagcccgaccgctgcgccttatccggtaactatcgtcttgagtccaacccg
gtaagacacgacttatcgccactggcagcagccactggtaacaggattagcagagcgaggtatgtaggcg
gtgctacagagttcttgaagtggtggcctaactacggctacactagaaggacagtatttggtatctgcgc
tctgctgaagccagttaccttcggaaaaagagttggtagctcttgatccggcaaacaaaccaccgctggt
agcggtggtttttttgtttgcaagcagcagattacgcgcagaaaaaaaggatctcaagaagatcctttga
tcttttctacggggtctgacgctcagtggaacgaaaactcacgttaagggattttggtcatgagattatc
aaaaaggatcttcacctagatccttttaaaggccggccgcggccgccatcggcattttcttttgcgtttt
tatttgttaactgttaattgtccttgttcaaggatgctgtctttgacaacagatgttttcttgcctttga
tgttcagcaggaagctcggcgcaaacgttgattgtttgtctgcgtagaatcctctgtttgtcatatagct
tgtaatcacgacattgtttcctttcgcttgaggtacagcgaagtgtgagtaagtaaaggttacatcgtta
ggatcaagatccatttttaacacaaggccagttttgttcagcggcttgtatgggccagttaaagaattag
aaacataaccaagcatgtaaatatcgttagacgtaatgccgtcaatcgtcatttttgatccgcgggagtc
agtgaacaggtaccatttgccgttcattttaaagacgttcgcgcgttcaatttcatctgttactgtgtta
gatgcaatcagcggtttcatcacttttttcagtgtgtaatcatcgtttagctcaatcataccgagagcgc
cgtttgctaactcagccgtgcgttttttatcgctttgcagaagtttttgactttcttgacggaagaatga
tgtgcttttgccatagtatgctttgttaaataaagattcttcgccttggtagccatcttcagttccagtg
tttgcttcaaatactaagtatttgtggcctttatcttctacgtagtgaggatctctcagcgtatggttgt
cgcctgagctgtagttgccttcatcgatgaactgctgtacattttgatacgtttttccgtcaccgtcaaa
gattgatttataatcctctacaccgttgatgttcaaagagctgtctgatgctgatacgttaacttgtgca
gttgtcagtgtttgtttgccgtaatgtttaccggagaaatcagtgtagaataaacggatttttccgtcag
atgtaaatgtggctgaacctgaccattcttgtgtttggtcttttaggatagaatcatttgcatcgaattt
gtcgctgtctttaaagacgcggccagcgtttttccagctgtcaatagaagtttcgccgactttttgatag
aacatgtaaatcgatgtgtcatccgcatttttaggatctccggctaatgcaaagacgatgtggtagccgt
gatagtttgcgacagtgccgtcagcgttttgtaatggccagctgtcccaaacgtccaggccttttgcaga
agagatatttttaattgtggacgaatcaaattcagaaacttgatatttttcatttttttgctgttcaggg
atttgcagcatatcatggcgtgtaatatgggaaatgccgtatgtttccttatatggcttttggttcgttt
ctttcgcaaacgcttgagttgcgcctcctgccagcagtgcggtagtaaaggttaatactgttgcttgttt
tgcaaactttttgatgttcatcgttcatgtctccttttttatgtactgtgttagcggtctgcttcttcca
gccctcctgtttgaagatggcaagttagttacgcacaataaaaaaagacctaaaatatgtaaggggtgac
gccaaagtatacactttgccctttacacattttaggtcttgcctgctttatcagtaacaaacccgcgcga
tttacttttcgacctcattctattagactctcgtttggattgcaactggtctattttcctcttttgtttg
atagaaaatcataaaaggatttgcagactacgggcctaaagaactaaaaaatctatctgtttcttttcat
tctctgtattttttatagtttctgttgcatgggcataaagttgcctttttaatcacaattcagaaaatat
cataatatctcatttcactaaataatagtgaacggcaggtatatgtgatgggttaaaaaggatcggcggc
cgctcgatttaaatc
SEQ ID NO: 14
(complete nucleotide sequence of plasmid pSacB_alaD)
(artificial)
tcgagtaagtgcatatgaatatgaaatacttcttgcccgccgtgtttgttacaattgacaattaaacggt
agccgtcttccgcaataccttccagtttggcaattttagcggcagtaataaataagcgccctaatacggc
ttcatcttctgcggttacgtcgtttactgtcggaatcaatttattcggaataattaaaatatgagttttt
gcctgcggcgcaatatcgcgaaatgcggtgacaagatcgtcttgatatataatgtcggcgggaatttctt
tacgaataattttactgaaaattgtttcttctgccattttgtgtttccttatttttgggaaaaatctacc
gcactttttatcagaaatcagcttaaatagcaatttatctcgtaaaccaaaggaataaatccacaccctt
tataatggtattattactctatttgggtaattttgatttaggtcaaaaaatctgtaaaaggtgatatgga
tcactcaaattagctattatctaatttatgaatcttttataatccccccgttaaataatattcaacaatt
ttggattttttaatctatcatttatgctttaaggcagttctactcatttccgagtagttttattactaag
gaaagctcaatgaaatcggaagattttaaattggcttggatggcttcgccaaccgagatggctcaaaccg
ggttagacgtcggcgtttataaagctacgaaaaaacaagcctattcatttttatcggcgatctctgccgg
tatgtttattgctcttgcattcgttttttatacaacaactcaaacagcctctgcgggagcgccttgggga
ttaactaaactggtcggcggtttggtgttctctctcggggtaattatggtggtggtttgcggctgtgaac
tatttacttcatcaactttatcgactattgcccgctttgagagtaaaattacaacaattcagatgttacg
taactggattgtggtttatttcggtaattttgtcggcggtttatttattgttgcattaatttggttttcc
ggtcagatcatggcggcaaacggtcagtggggattaaccattttaaatacggcacaacataaaatagaac
atacctggattgaagccttctgtttaggtattctttgcaacattatggtatgtattgccgtttggatggc
ctatgccggcaaaactctaacggataaagcttttattatgatcctgccgatcgggttatttgtcgcttca
ggctttgaacactgcgtagcaaatatgtttatgatccctatgggcatggtaattgcaaatttcgcatcgc
cggaattctggcaggcaacgggtttaaatgccgagcagtttgcaaatttagatatgtaccatttagtaat
taaaaatttaattcctgttactttaggtaacatcgtcggtggtggtgtttgcattggtctaatgcaatgg
tttaccagtcgtccacattagttgggtgagagtgacggcaaatccgccgtcatccttgcaaggtttcaat
cttatcaatactagaaaagaaggaagtattaaaaatgaaaattggcatccctaaagagattaagaacaat
gaaaaccgtgtagcaatcaccccggcaggtgttatgactctggttaaagcgggccacgatgtgtacgtcg
aaaccgaagcgggtgccggcagcggcttcagcgacagcgagtatgagaaggcgggtgcggttattgtgac
taaggcggaggacgcttgggcagccgaaatggttctgaaggtgaaagaaccgctggcggaggagtttcgc
tattttcgtccgggtctgattttgttcacctacctgcacctggctgcggccgaggcgctgaccaaggcac
tggtggagcagaaggttgttggcatcgcgtacgaaacggttcaactggcgaatggttccctgccgctgct
gacccctatgtctgaagttgcgggtcgcatgagcgttcaagtcggcgctcagtttctggagaaaccgcac
ggtggcaagggcattttgctgggtggtgttccgggtgtccgccgtggtaaagtgacgatcattggcggtg
gtacggccggtacgaacgcggccaagattgccgtaggtctgggtgcagatgtgaccattctggacatcaa
cgcggaacgtttgcgtgagctggacgatctgtttggcgaccaagtcaccaccctgatgagcaacagctac
cacatcgcggagtgcgtccgtgaaagcgatttggtcgttggtgcggtgctgatcccgggtgcaaaagccc
cgaaactggtgaccgaggagatggtccgtagcatgaccccgggttcggttctggtcgacgtggcaattga
ccagggcggtatcttcgaaaccaccgaccgcgtcacgacccatgatgacccgacctatgtgaaacatggc
gtggttcactatgcggtcgcgaatatgccgggtgcagtgccgcgcacgtccacgttcgcgctgacgaacg
tgacgattccatacgctctgcagatcgccaataagggctatcgtgcggcgtgtctggataatccggcatt
gctgaaaggcatcaataccctggatggtcatatcgtttacgaggctgtggctgcagcacacaacatgccg
tacactgatgtccatagcttgctgcaaggctaattgagagtttgtcttattgcttaataaattccgcctc
aataggcggaatttttttgttttaattcccctgattaaagcggataaaagtgcggtagttttttgcgaag
atttgactattctctgaaaaaaacgaaattctttgctataatcttcttgctatattttgttgattattta
agggcatattatgtcggttttaggacgaattcattcatttgaaacctgcgggacagttgacgggccggga
atccgctttattttatttttacaaggctgcttaatgcgttgtaaatactgccataatagagacacctggg
atttgcacggcggtaaagaaatttccgttgaagaattaatgaaagaagtggtgacctatcgccattttat
gaacgcctcgggcggcggagttaccgcttccggcggtgaagctattttacaggcggaatttgtacgggac
tggttcagagcctgccataaagaaggaattaatacttgcttggataccaacggtttcgtccgtcatcatg
atcatattattgatgaattgattgatgacacggatcttgtgttgcttgacctgaaagaaatgaatgaacg
ggttcacgaaagcctgattggcgtgccgaataaaagagtgctcgaattcgcaaaatatttagcggatcga
aatcagcgtacctggatccgccatgttgtagtgccgggttatacagatagtgacgaagatttgcacatgc
tggggaatttcattaaagatatgaagaatatcgaaaaagtggaattattaccttatcaccgtctaggcgc
ccataaatgggaagtactcggcgataaatacgagcttgaagatgtaaaaccgccgacaaaagaattaatg
gagcatgttaaggggttgcttgcaggctacgggcttaatgtgacatattagaagaaataaaaaaaccgtc
gtaaacattatgacggtttttttgtcactatttttcagaggagttaagccgggggtgttgtaaaagtgcg
gtagctttttgttgttttttctgttccctgcgcttttggaaaaagcggcttaacttctgactgcattgat
cctgtaagacaccgcttgtgatctcaaccccatgattcattttataatcctcaaaaaaatgaaatctgga
acccaccgcaccggttttgtaatcggacgccccgaataccaagcgtttgattcggctgtgtaaaatcgcg
ccggcgcacatggtgcagggttctaaagtcacgtataaagtggtattgagcaggcggtaattttggattt
tctgcgcggcgttacgcaacgcaataatttcggcatgggcggtgggatccgagttcacaatagagaggtt
ccagccttcaccaatgatattgccccgttcatccaccaatacggcacctacgggaatttcccctaaagct
tccgccttgtcggcaaggaaaagagctcgattcatcattttttcgtcaaagctaatttgttgatctagac
tccataggccgctttcctggctttgcttccagatgtatgctctcctccggagagtaccgtgactttattt
tcggcacaaatacaggggtcgatggataaatacggcgatagtttcctgacggatgatccgtatgtaccgg
cggaagacaagctgcaaacctgtcagatggagattgatttaatggcggatgtgctgagagcaccgccccg
tgaatccgcagaactgatccgctatgtgtttgcggatgattggccggaataaataaagccgggcttaata
cagattaagcccgtatagggtattattactgaataccaaacagcttacggaggacggaatgttacccatt
gagacaaccagactgccttctgattattaatatttttcactattaatcagaaggaataaccatgaatttt
acccggattgacctgaatacctggaatcgcagggaacactttgccctttatcgtcagcagattaaatgcg
gattcagcctgaccaccaaactcgatattaccgctttgcgtaccgcactggcggagacaggttataagtt
ttatccgctgatgatttacctgatctcccgggctgttaatcagtttccggagttccggatggcactgaaa
gacaatgaacttatttactgggaccagtcagacccggtctttactgtctttcataaagaaaccgaaacat
tctctgcactgtcctgccgttattttccggatctcagtgagtttatggcaggttataatgcggtaacggc
agaatatcagcatgataccagattgtttccgcagggaaatttaccggagaatcacctgaatatatcatca
ttaccgtgggtgagttttgacgggatttaacctgaacatcaccggaaatgatgattattttgccccggtt
tttacgatggcaaagtttcagcaggaaggtgaccgcgtattattacctgtttctgtacaggttcatcatg
cagtctgtgatggctttcatgcagcacggtttattaatacacttcagctgatgtgtgataacatactgaa
ataaattaattaattctgtatttaagccaccgtatccggcaggaatggtggctttttttttatattttaa
ccgtaatctgtaatttcgtttcagactggttcaggatgagctcgcttggactcctgttgatagatccagt
aatgacctcagaactccatctggatttgttcagaacgctcggttgccgccgggcgttttttattggtgag
aatccaagcactagcggcgcgccggccggcccggtgtgaaataccgcacagatgcgtaaggagaaaatac
cgcatcaggcgctcttccgcttcctcgctcactgactcgctgcgctcggtcgttcggctgcggcgagcgg
tatcagctcactcaaaggcggtaatacggttatccacagaatcaggggataacgcaggaaagaacatgtg
agcaaaaggccagcaaaaggccaggaaccgtaaaaaggccgcgttgctggcgtttttccataggctccgc
ccccctgacgagcatcacaaaaatcgacgctcaagtcagaggtggcgaaacccgacaggactataaagat
accaggcgtttccccctggaagctccctcgtgcgctctcctgttccgaccctgccgcttaccggatacct
gtccgcctttctcccttcgggaagcgtggcgctttctcatagctcacgctgtaggtatctcagttcggtg
taggtcgttcgctccaagctgggctgtgtgcacgaaccccccgttcagcccgaccgctgcgccttatccg
gtaactatcgtcttgagtccaacccggtaagacacgacttatcgccactggcagcagccactggtaacag
gattagcagagcgaggtatgtaggcggtgctacagagttcttgaagtggtggcctaactacggctacact
agaaggacagtatttggtatctgcgctctgctgaagccagttaccttcggaaaaagagttggtagctctt
gatccggcaaacaaaccaccgctggtagcggtggtttttttgtttgcaagcagcagattacgcgcagaaa
aaaaggatctcaagaagatcctttgatcttttctacggggtctgacgctcagtggaacgaaaactcacgt
taagggattttggtcatgagattatcaaaaaggatcttcacctagatccttttaaaggccggccgcggcc
gccatcggcattttcttttgcgtttttatttgttaactgttaattgtccttgttcaaggatgctgtcttt
gacaacagatgttttcttgcctttgatgttcagcaggaagctcggcgcaaacgttgattgtttgtctgcg
tagaatcctctgtttgtcatatagcttgtaatcacgacattgtttcctttcgcttgaggtacagcgaagt
gtgagtaagtaaaggttacatcgttaggatcaagatccatttttaacacaaggccagttttgttcagcgg
cttgtatgggccagttaaagaattagaaacataaccaagcatgtaaatatcgttagacgtaatgccgtca
atcgtcatttttgatccgcgggagtcagtgaacaggtaccatttgccgttcattttaaagacgttcgcgc
gttcaatttcatctgttactgtgttagatgcaatcagcggtttcatcacttttttcagtgtgtaatcatc
gtttagctcaatcataccgagagcgccgtttgctaactcagccgtgcgttttttatcgctttgcagaagt
ttttgactttcttgacggaagaatgatgtgcttttgccatagtatgctttgttaaataaagattcttcgc
cttggtagccatcttcagttccagtgtttgcttcaaatactaagtatttgtggcctttatcttctacgta
gtgaggatctctcagcgtatggttgtcgcctgagctgtagttgccttcatcgatgaactgctgtacattt
tgatacgtttttccgtcaccgtcaaagattgatttataatcctctacaccgttgatgttcaaagagctgt
ctgatgctgatacgttaacttgtgcagttgtcagtgtttgtttgccgtaatgtttaccggagaaatcagt
gtagaataaacggatttttccgtcagatgtaaatgtggctgaacctgaccattcttgtgtttggtctttt
aggatagaatcatttgcatcgaatttgtcgctgtctttaaagacgcggccagcgtttttccagctgtcaa
tagaagtttcgccgactttttgatagaacatgtaaatcgatgtgtcatccgcatttttaggatctccggc
taatgcaaagacgatgtggtagccgtgatagtttgcgacagtgccgtcagcgttttgtaatggccagctg
tcccaaacgtccaggccttttgcagaagagatatttttaattgtggacgaatcaaattcagaaacttgat
atttttcatttttttgctgttcagggatttgcagcatatcatggcgtgtaatatgggaaatgccgtatgt
ttccttatatggcttttggttcgtttctttcgcaaacgcttgagttgcgcctcctgccagcagtgcggta
gtaaaggttaatactgttgcttgttttgcaaactttttgatgttcatcgttcatgtctccttttttatgt
actgtgttagcggtctgcttcttccagccctcctgtttgaagatggcaagttagttacgcacaataaaaa
aagacctaaaatatgtaaggggtgacgccaaagtatacactttgccctttacacattttaggtcttgcct
gctttatcagtaacaaacccgcgcgatttacttttcgacctcattctattagactctcgtttggattgca
actggtctattttcctcttttgtttgatagaaaatcataaaaggatttgcagactacgggcctaaagaac
taaaaaatctatctgtttcttttcattctctgtattttttatagtttctgttgcatgggcataaagttgc
ctttttaatcacaattcagaaaatatcataatatctcatttcactaaataatagtgaacggcaggtatat
gtgatgggttaaaaaggatcggcggccgctcgatttaaatc
SEQ ID NO: 15
(complete nucleotide sequence of plasmid pSacB_delta_IdhA)
(artificial)
tcgagaggcctgacgtcgggcccggtaccacgcgtcatatgactagttcggacctagggatgggtcagcc
tgaacgaaccgcacttgtatgtaggtagttttgaccgcccgaatattcgttataccttggtggaaaaatt
caaaccgatggagcaattatacaattttgtggcggcgcaaaaaggtaaaagcggtatcgtctattgcaac
agccgtagcaaagtggagcgcattgcggaagccctgaagaaaagaggcatttccgcagccgcttatcatg
cgggcatggagccgtcgcagcgggaagcggtgcaacaggcgtttcaacgggataatattcaagtggtggt
ggcgaccattgcttttggtatggggatcaacaaatctaatgtgcgttttgtggcgcattttgatttatct
cgcagcattgaggcgtattatcaggaaaccgggcgcgcggggcgggacgacctgccggcggaagcggtac
tgttttacgagccggcggattatgcctggttgcataaaattttattggaagagccggaaagcccgcaacg
ggatattaaacggcataagctggaagccatcggcgaatttgccgaaagccagacctgccgtcgtttagtg
ctgttaaattatttcggcgaaaaccgccaaacgccatgtaataactgtgatatctgcctcgatccgccga
aaaaatatgacggattattagacgcgcagaaaatcctttcgaccatttatcgcaccgggcaacgtttcgg
cacgcaatacgtaatcggcgtaatgcgcggtttgcagaatcagaaaataaaagaaaatcaacatgatgag
ttgaaagtctacggaattggcaaagataaaagcaaagaatactggcaatcggtaattcgtcagctgattc
atttgggctttgtgcaacaaatcatcagcgatttcggcatggggaccagattacagctcaccgaaagcgc
gcgtcccgtgctgcgcggcgaagtgtctttggaactggccatgccgagattatcttccattaccatggta
caggctccgcaacgcaatgcggtaaccaactacgacaaagatttatttgcccgcctgcgtttcctgcgca
aacagattgccgacaaagaaaacattccgccttatattgtgttcagtgacgcgaccttgcaggaaatgtc
gttgtatcagccgaccagcaaagtggaaatgctgcaaatcaacggtgtcggcgccatcaaatggcagcgc
ttcggacagccttttatggcgattattaaagaacatcaggctttgcgtaaagcgggtaagaatccgttgg
aattgcaatcttaaaatttttaactttttgaccgcacttttaaggttagcaaattccaataaaaagtgcg
gtgggttttcgggaatttttaacgcgctgatttcctcgtcttttcaatttyttcgyctccatttgttcgg
yggttgccggatcctttcttgactgagatccataagagagtagaatagcgccgcttatatttttaatagc
gtacctaatcgggtacgctttttttatgcggaaaatccatatttttctaccgcactttttctttaaagat
ttatacttaagtctgtttgattcaatttatttggaggttttatgcaacacattcaactggctcccgattt
aacattcagtcgcttaattcaaggattctggcggttaaaaagctggcggaaatcgccgcaggaattgctt
acattcgttaagcaaggattagaattaggcgttgatacgctggatcatgccgcttgttacggggctttta
cttccgaggcggaattcggacgggcgctggcgctggataaatccttgcgcgcacagcttactttggtgac
caaatgcgggattttgtatcctaatgaagaattacccgatataaaatcccatcactatgacaacagctac
cgccatattatgtggtcggcgcaacgttccattgaaaaactgcaatgcgactatttagatgtattgctga
ttcaccgwctttctccctgtgcggatcccgaacaaatcgcgcgggcttttgatgaactttatcaaaccgg
raaagtacgttatttcggggtatctaactatacgccggctaagttcgccatgttgcaatcttatgtgaat
cagccgttaatcactaatcaaattgagatttcgcctcttcatcgtcaggcttttgatgacggtaccctgg
attttttactggaaaaacgtattcaaccgatggcatggtcgccacttgccggcggtcgtttattcaatca
ggatgagaacagtcgggcggtgcaaaaaacattactcgaaatcggtgaaacgaaaggagaaacccgttta
gatacattggcttatgcctggttattggcgcatccggcaaaaattatgccggttatggggtccggtaaaa
ttgaacgggtaaaaagcgcggcggatgcgttacgaatttccttcactgaggaagaatggattaaggttta
tgttgccgcacagggacgggatattccgtaacatcatccgtctaatcctgcgtatctggggaaagatgcg
tcatcgtaagaggtctataatattcgtcgttttgataagggtgccatatccggcacccgttaaaatcaca
ttgcgttcgcaacaaaattattccttacgaatagcattcacctcttttaacagatgttgaatatccgtat
cggcaaaaatatcctctatatttgcggttaaacggcgccgccagttagcatattgagtgctggttcccgg
aatattgacgggttcggtcataccgagccagtcttcaggttggaatccccatcgtcgacatcgatgctct
tctgcgttaattaacaattgggatcctctagactccataggccgctttcctggctttgcttccagatgta
tgctctcctccggagagtaccgtgactttattttcggcacaaatacaggggtcgatggataaatacggcg
atagtttcctgacggatgatccgtatgtaccggcggaagacaagctgcaaacctgtcagatggagattga
tttaatggcggatgtgctgagagcaccgccccgtgaatccgcagaactgatccgctatgtgtttgcggat
gattggccggaataaataaagccgggcttaatacagattaagcccgtatagggtattattactgaatacc
aaacagcttacggaggacggaatgttacccattgagacaaccagactgccttctgattattaatattttt
cactattaatcagaaggaataaccatgaattttacccggattgacctgaatacctggaatcgcagggaac
actttgccctttatcgtcagcagattaaatgcggattcagcctgaccaccaaactcgatattaccgcttt
gcgtaccgcactggcggagacaggttataagttttatccgctgatgatttacctgatctcccgggctgtt
aatcagtttccggagttccggatggcactgaaagacaatgaacttatttactgggaccagtcagacccgg
tctttactgtctttcataaagaaaccgaaacattctctgcactgtcctgccgttattttccggatctcag
tgagtttatggcaggttataatgcggtaacggcagaatatcagcatgataccagattgtttccgcaggga
aatttaccggagaatcacctgaatatatcatcattaccgtgggtgagttttgacgggatttaacctgaac
atcaccggaaatgatgattattttgccccggtttttacgatggcaaagtttcagcaggaaggtgaccgcg
tattattacctgtttctgtacaggttcatcatgcagtctgtgatggctttcatgcagcacggtttattaa
tacacttcagctgatgtgtgataacatactgaaataaattaattaattctgtatttaagccaccgtatcc
ggcaggaatggtggctttttttttatattttaaccgtaatctgtaatttcgtttcagactggttcaggat
gagctcgcttggactcctgttgatagatccagtaatgacctcagaactccatctggatttgttcagaacg
ctcggttgccgccgggcgttttttattggtgagaatccaagcactagcggcgcgccggccggcccggtgt
gaaataccgcacagatgcgtaaggagaaaataccgcatcaggcgctcttccgcttcctcgctcactgact
cgctgcgctcggtcgttcggctgcggcgagcggtatcagctcactcaaaggcggtaatacggttatccac
agaatcaggggataacgcaggaaagaacatgtgagcaaaaggccagcaaaaggccaggaaccgtaaaaag
gccgcgttgctggcgtttttccataggctccgcccccctgacgagcatcacaaaaatcgacgctcaagtc
agaggtggcgaaacccgacaggactataaagataccaggcgtttccccctggaagctccctcgtgcgctc
tcctgttccgaccctgccgcttaccggatacctgtccgcctttctcccttcgggaagcgtggcgctttct
catagctcacgctgtaggtatctcagttcggtgtaggtcgttcgctccaagctgggctgtgtgcacgaac
cccccgttcagcccgaccgctgcgccttatccggtaactatcgtcttgagtccaacccggtaagacacga
cttatcgccactggcagcagccactggtaacaggattagcagagcgaggtatgtaggcggtgctacagag
ttcttgaagtggtggcctaactacggctacactagaaggacagtatttggtatctgcgctctgctgaagc
cagttaccttcggaaaaagagttggtagctcttgatccggcaaacaaaccaccgctggtagcggtggttt
ttttgtttgcaagcagcagattacgcgcagaaaaaaaggatctcaagaagatcctttgatcttttctacg
gggtctgacgctcagtggaacgaaaactcacgttaagggattttggtcatgagattatcaaaaaggatct
tcacctagatccttttaaaggccggccgcggccgccatcggcattttcttttgcgtttttatttgttaac
tgttaattgtccttgttcaaggatgctgtctttgacaacagatgttttcttgcctttgatgttcagcagg
aagctcggcgcaaacgttgattgtttgtctgcgtagaatcctctgtttgtcatatagcttgtaatcacga
cattgtttcctttcgcttgaggtacagcgaagtgtgagtaagtaaaggttacatcgttaggatcaagatc
catttttaacacaaggccagttttgttcagcggcttgtatgggccagttaaagaattagaaacataacca
agcatgtaaatatcgttagacgtaatgccgtcaatcgtcatttttgatccgcgggagtcagtgaacaggt
accatttgccgttcattttaaagacgttcgcgcgttcaatttcatctgttactgtgttagatgcaatcag
cggtttcatcacttttttcagtgtgtaatcatcgtttagctcaatcataccgagagcgccgtttgctaac
tcagccgtgcgttttttatcgctttgcagaagtttttgactttcttgacggaagaatgatgtgcttttgc
catagtatgctttgttaaataaagattcttcgccttggtagccatcttcagttccagtgtttgcttcaaa
tactaagtatttgtggcctttatcttctacgtagtgaggatctctcagcgtatggttgtcgcctgagctg
tagttgccttcatcgatgaactgctgtacattttgatacgtttttccgtcaccgtcaaagattgatttat
aatcctctacaccgttgatgttcaaagagctgtctgatgctgatacgttaacttgtgcagttgtcagtgt
ttgtttgccgtaatgtttaccggagaaatcagtgtagaataaacggatttttccgtcagatgtaaatgtg
gctgaacctgaccattcttgtgtttggtcttttaggatagaatcatttgcatcgaatttgtcgctgtctt
taaagacgcggccagcgtttttccagctgtcaatagaagtttcgccgactttttgatagaacatgtaaat
cgatgtgtcatccgcatttttaggatctccggctaatgcaaagacgatgtggtagccgtgatagtttgcg
acagtgccgtcagcgttttgtaatggccagctgtcccaaacgtccaggccttttgcagaagagatatttt
taattgtggacgaatcaaattcagaaacttgatatttttcatttttttgctgttcagggatttgcagcat
atcatggcgtgtaatatgggaaatgccgtatgtttccttatatggcttttggttcgtttctttcgcaaac
gcttgagttgcgcctcctgccagcagtgcggtagtaaaggttaatactgttgcttgttttgcaaactttt
tgatgttcatcgttcatgtctccttttttatgtactgtgttagcggtctgcttcttccagccctcctgtt
tgaagatggcaagttagttacgcacaataaaaaaagacctaaaatatgtaaggggtgacgccaaagtata
cactttgccctttacacattttaggtcttgcctgctttatcagtaacaaacccgcgcgatttacttttcg
acctcattctattagactctcgtttggattgcaactggtctattttcctcttttgtttgatagaaaatca
taaaaggatttgcagactacgggcctaaagaactaaaaaatctatctgtttcttttcattctctgtattt
tttatagtttctgttgcatgggcataaagttgcctttttaatcacaattcagaaaatatcataatatctc
atttcactaaataatagtgaacggcaggtatatgtgatgggttaaaaaggatcggcggccgctcgattta
aatc
SEQ ID NO: 16 (complete nucleotide sequence of plasmid pSacB_delta_pflD)
(artificial)
tcgagaggcctgacgtcgggcccggtaccacgcgtcatatgactagttcggacctagggatgggatcgag
ctcttttccttgccgacaaggcggaagctttaggggaaattcccgtaggtgccgtattggtggatgaacg
gggcaatatcattggtgaaggctggaacctctctattgtgaactcggatcccaccgcccatgccgaaatt
attgcgttgcgtaacgccgcgcagaaaatccaaaattaccgcctgctcaataccactttatacgtgactt
tagaaccctgcaccatgtgcgccggcgcgattttacacagccgaatcaaacgcttggtattcggggcgtc
cgattacaaaaccggtgcggtgggttccagatttcatttttttgaggattataaaatgaatcatggggtt
gagatcacaagcggtgtcttataggatcaatgcagtcagaagttaagccgctttttccaaaagcgcaggg
aacagaaaaaacaacaaaaagctaccgcacttttacaacacccccggcttaactcctctgaaaaatagtg
acaaaaaaaccgtcataatgtttacgacggtttttttatttcttctaatatgtcacattaagcccgtagc
ctgcaagcaaccccttaacatgctccattaattcttttgtcggcggttttacatcttcaagctcgtattt
atcgccgagtacttcccatttatgggcgcctagacggtgataaggtaataattccactttttcgatattc
ttcatatctttaatgaaattccccagcatgtgcaaatcttcgtcactatctgtataacccggcactacaa
catggcggatccaggtacgctgatttcgatccgctaaatattttgcgaattcgagcactcttttattcgg
cacgccaatcaggctttcgtgaacccgttcattcatttctttcaggtcaagcaacacaagatccgtgtca
tcaatcaattcatcaataatatgatcatgatgacggacgaaaccgttggtatccaagcaagtattaattc
cttctttatggcaggctctgaaccagtcccgtacaaattccgcctgtaaaatagcttcaccgccggaagc
ggtaactccgccgcccgaggcgttcataaaatggcgataggtcaccacttctttcattaattcttcaacg
gaaatttctttaccgccgtgcaaatcccaggtgtctctgttatggcaatatttacaacgcattaagcagc
cttgtaaaaataaaataaagcggattcccggcccgtcaactgtcccgcaggtttcaaatgaatgaattcg
tcctaaaaccgacataatatgcccttaaataatcaacaaaatatagcaagaagattatagcaaagaattt
cgtttttttcagagaatagtcaaatcttcgcaaaaaactaccgcacttttatccgctttaatcaggggaa
ttaaaacaaaaaaattccgcctattgaggcggaatttattaagcaataagacaaactctcaattttaata
cttccttcttttctagtattgataagattgaaaccttgcaaggatgacggcggatttgccgtcactctca
cccaactaatgtggacgactggtaaaccattgcattagaccaatgcaaacaccaccaccgacgatgttac
ctaaagtaacaggaattaaatttttaattactaaatggtacatatctaaatttgcaaactgctcggcatt
taaacccgttgcctgccagaattccggcgatgcgaaatttgcaattaccatgcccatagggatcataaac
atatttgctacgcagtgttcaaagcctgaagcgacaaayaacccgatcggcaggatcataataaaagctt
tatccgttagagtyttgccggcataggccatccaaacggcaatacataccataatgttgcaaagaatacc
taaacagaaggcttcaayccaggtatgttctattttatgttgtgccgtatttaaaatggttaatccccac
tgaccgtttgccgccatgatctgaccggaaaaccaaattaatgcaacaataaataaaccgccgacaaaat
taccgaartaaaccacaatccagttacgtaacatctgaattgttgtaattttactctcaaagcgggcaat
agtcgataaagttgatgaagtaaatagttcacagccgcaaaccgccaccataattaccccgagagagaac
accaaaccgccgaccagtttagttaatccccaaggcgctcccgcagaggctgtttgagttgttgtataaa
aaacgaatgcaagagcaataaacataccggcagagatcgccgataaaaatgaataggcttgttttttcgt
agctttataaacgccgacgtctaacccggtttgagccatctcggttggcgaagccatccaagccaattta
aaatcttccgatttcattgagctttccttagtaataaaactactcggaaatgagtagaactgccttaaag
cataaatgatagattaaaaaatccaaaattgttgaatattatttaacggggggattataaaagattcata
aattagataatagctaatttgagtgatccatatcaccttttacagattttttgacctaaatcaaaattac
ccaaatagagtaataataccattataaagggtgtggatttattcctttggtttacgagataaattgctat
ttaagctgatttctgataaaaagtgcggtagatttttcccaaaaataaggaaacacaaaatggcagaaga
aacaattttcagtaaaattattcgtaaagaaattcccgccgacattatatatcaagacgatcttgtcacc
gcatttcgcgatattgcgccgcaggcaaaaactcatattttaattattccgaataaattgattccgacag
taaacgacgtaaccgcccatcgtcgacatcgatgctcttctgcgttaattaacaattgggatcctctaga
ctttgcttccagatgtatgctctcctccggagagtaccgtgactttattttcggcacaaatacaggggtc
gatggataaatacggcgatagtttcctgacggatgatccgtatgtaccggcggaagacaagctgcaaacc
tgtcagatggagattgatttaatggcggatgtgctgagagcaccgccccgtgaatccgcagaactgatcc
gctatgtgtttgcggatgattggccggaataaataaagccgggcttaatacagattaagcccgtataggg
tattattactgaataccaaacagcttacggaggacggaatgttacccattgagacaaccagactgccttc
tgattattaatatttttcactattaatcagaaggaataaccatgaattttacccggattgacctgaatac
ctggaatcgcagggaacactttgccctttatcgtcagcagattaaatgcggattcagcctgaccaccaaa
ctcgatattaccgctttgcgtaccgcactggcggagacaggttataagttttatccgctgatgatttacc
tgatctcccgggctgttaatcagtttccggagttccggatggcactgaaagacaatgaacttatttactg
ggaccagtcagacccggtctttactgtctttcataaagaaaccgaaacattctctgcactgtcctgccgt
tattttccggatctcagtgagtttatggcaggttataatgcggtaacggcagaatatcagcatgatacca
gattgtttccgcagggaaatttaccggagaatcacctgaatatatcatcattaccgtgggtgagttttga
cgggatttaacctgaacatcaccggaaatgatgattattttgccccggtttttacgatggcaaagtttca
gcaggaaggtgaccgcgtattattacctgtttctgtacaggttcatcatgcagtctgtgatggctttcat
gcagcacggtttattaatacacttcagctgatgtgtgataacatactgaaataaattaattaattctgta
tttaagccaccgtatccggcaggaatggtggctttttttttatattttaaccgtaatctgtaatttcgtt
tcagactggttcaggatgagctcgcttggactcctgttgatagatccagtaatgacctcagaactccatc
tggatttgttcagaacgctcggttgccgccgggcgttttttattggtgagaatccaagcactagcggcgc
gccggccggcccggtgtgaaataccgcacagatgcgtaaggagaaaataccgcatcaggcgctcttccgc
ttcctcgctcactgactcgctgcgctcggtcgttcggctgcggcgagcggtatcagctcactcaaaggcg
gtaatacggttatccacagaatcaggggataacgcaggaaagaacatgtgagcaaaaggccagcaaaagg
ccaggaaccgtaaaaaggccgcgttgctggcgtttttccataggctccgcccccctgacgagcatcacaa
aaatcgacgctcaagtcagaggtggcgaaacccgacaggactataaagataccaggcgtttccccctgga
agctccctcgtgcgctctcctgttccgaccctgccgcttaccggatacctgtccgcctttctcccttcgg
gaagcgtggcgctttctcatagctcacgctgtaggtatctcagttcggtgtaggtcgttcgctccaagct
gggctgtgtgcacgaaccccccgttcagcccgaccgctgcgccttatccggtaactatcgtcttgagtcc
aacccggtaagacacgacttatcgccactggcagcagccactggtaacaggattagcagagcgaggtatg
taggcggtgctacagagttcttgaagtggtggcctaactacggctacactagaaggacagtatttggtat
ctgcgctctgctgaagccagttaccttcggaaaaagagttggtagctcttgatccggcaaacaaaccacc
gctggtagcggtggtttttttgtttgcaagcagcagattacgcgcagaaaaaaaggatctcaagaagatc
ctttgatcttttctacggggtctgacgctcagtggaacgaaaactcacgttaagggattttggtcatgag
attatcaaaaaggatcttcacctagatccttttaaaggccggccgcggccgccatcggcattttcttttg
cgtttttatttgttaactgttaattgtccttgttcaaggatgctgtctttgacaacagatgttttcttgc
ctttgatgttcagcaggaagctcggcgcaaacgttgattgtttgtctgcgtagaatcctctgtttgtcat
atagcttgtaatcacgacattgtttcctttcgcttgaggtacagcgaagtgtgagtaagtaaaggttaca
tcgttaggatcaagatccatttttaacacaaggccagttttgttcagcggcttgtatgggccagttaaag
aattagaaacataaccaagcatgtaaatatcgttagacgtaatgccgtcaatcgtcatttttgatccgcg
ggagtcagtgaacaggtaccatttgccgttcattttaaagacgttcgcgcgttcaatttcatctgttact
gtgttagatgcaatcagcggtttcatcacttttttcagtgtgtaatcatcgtttagctcaatcataccga
gagcgccgtttgctaactcagccgtgcgttttttatcgctttgcagaagtttttgactttcttgacggaa
gaatgatgtgcttttgccatagtatgctttgttaaataaagattcttcgccttggtagccatcttcagtt
ccagtgtttgcttcaaatactaagtatttgtggcctttatcttctacgtagtgaggatctctcagcgtat
ggttgtcgcctgagctgtagttgccttcatcgatgaactgctgtacattttgatacgtttttccgtcacc
gtcaaagattgatttataatcctctacaccgttgatgttcaaagagctgtctgatgctgatacgttaact
tgtgcagttgtcagtgtttgtttgccgtaatgtttaccggagaaatcagtgtagaataaacggatttttc
cgtcagatgtaaatgtggctgaacctgaccattcttgtgtttggtcttttaggatagaatcatttgcatc
gaatttgtcgctgtctttaaagacgcggccagcgtttttccagctgtcaatagaagtttcgccgactttt
tgatagaacatgtaaatcgatgtgtcatccgcatttttaggatctccggctaatgcaaagacgatgtggt
agccgtgatagtttgcgacagtgccgtcagcgttttgtaatggccagctgtcccaaacgtccaggccttt
tgcagaagagatatttttaattgtggacgaatcaaattcagaaacttgatatttttcatttttttgctgt
tcagggatttgcagcatatcatggcgtgtaatatgggaaatgccgtatgtttccttatatggcttttggt
tcgtttctttcgcaaacgcttgagttgcgcctcctgccagcagtgcggtagtaaaggttaatactgttgc
ttgttttgcaaactttttgatgttcatcgttcatgtctccttttttatgtactgtgttagcggtctgctt
cttccagccctcctgtttgaagatggcaagttagttacgcacaataaaaaaagacctaaaatatgtaagg
ggtgacgccaaagtatacactttgccctttacacattttaggtcttgcctgctttatcagtaacaaaccc
gcgcgatttacttttcgacctcattctattagactctcgtttggattgcaactggtctattttcctcttt
tgtttgatagaaaatcataaaaggatttgcagactacgggcctaaagaactaaaaaatctatctgtttct
tttcattctctgtattttttatagtttctgttgcatgggcataaagttgcctttttaatcacaattcaga
aaatatcataatatctcatttcactaaataatagtgaacggcaggtatatgtgatgggttaaaaaggatc
ggcggccgctcgatttaaatc
SEQ ID NO: 17 (complete nucleotide sequence of plasmid pSacB_delta_pflA)
(artificial)
tttttggtcacgaccgtgcattgggtttgcacccggtccgaatggcgcgcctgctcgacgaccgtccgga
gtattaccggttttcttaccgtataccacgttagaagtgatagtcaggatagattgtgtcggagttgcgt
tgcggtaagttttgtgtttttgaacttttttcatgaaacgttcaactaagtctaccgctaaatcatcaac
acgcggatcattgttaccgaattgcggatattcgccttcaatttcgaagtcgatagcaacattcgaggcc
acgacattaccgtctttatctttgatgtcgccgcgaatcggtttaactttcgcatatttgattgcggata
atgagtccgcagccacggaaagacccgcgataccgcaagccattgtacggaatacgtcgcgatcgtggaa
cgccatcaatgccgcttcatatgcatatttatcgtgcatgaagtggatgatgttcaatgcggttacatat
tgagtcgccaaccagtccatgaaactgtccatacgttcgattacggtatcgaaattcaatacttcgtctg
taatcggcgcagttttaggaccgacttgcataccatttttctcatcgataccgccgttaattgcgtataa
catagttttagctaagtttgcgcgcgcaccgaagaattgcatttgtttacctacgaccatcggtgatacg
cagcatgcgattgcatagtcatcgttgttgaagtcaggacgcattaagtcatcattttcgtattgtacgg
aggaagtatcaatagatactttcgcacagaaacgtttgaacgcttcaggtaattgttcggaccaaagaat
agttaagtttggttccggagaagtacccatagtgtataaagtatgtaatacgcggaagctgtttttagtt
accaacggacgaccgtctaagcccataccggcgatagtttcggttgccctctagactccataggccgctt
tcctggctttgcttccagatgtatgctctcctccggagagtaccgtgactttattttcggcacaaataca
ggggtcgatggataaatacggcgatagtttcctgacggatgatccgtatgtaccggcggaagacaagctg
caaacctgtcagatggagattgatttaatggcggatgtgctgagagcaccgccccgtgaatccgcagaac
tgatccgctatgtgtttgcggatgattggccggaataaataaagccgggcttaatacagattaagcccgt
atagggtattattactgaataccaaacagcttacggaggacggaatgttacccattgagacaaccagact
gccttctgattattaatatttttcactattaatcagaaggaataaccatgaattttacccggattgacct
gaatacctggaatcgcagggaacactttgccctttatcgtcagcagattaaatgcggattcagcctgacc
accaaactcgatattaccgctttgcgtaccgcactggcggagacaggttataagttttatccgctgatga
tttacctgatctcccgggctgttaatcagtttccggagttccggatggcactgaaagacaatgaacttat
ttactgggaccagtcagacccggtctttactgtctttcataaagaaaccgaaacattctctgcactgtcc
tgccgttattttccggatctcagtgagtttatggcaggttataatgcggtaacggcagaatatcagcatg
ataccagattgtttccgcagggaaatttaccggagaatcacctgaatatatcatcattaccgtgggtgag
ttttgacgggatttaacctgaacatcaccggaaatgatgattattttgccccggtttttacgatggcaaa
gtttcagcaggaaggtgaccgcgtattattacctgtttctgtacaggttcatcatgcagtctgtgatggc
tttcatgcagcacggtttattaatacacttcagctgatgtgtgataacatactgaaataaattaattaat
tctgtatttaagccaccgtatccggcaggaatggtggctttttttttatattttaaccgtaatctgtaat
ttcgtttcagactggttcaggatgagctcgcttggactcctgttgatagatccagtaatgacctcagaac
tccatctggatttgttcagaacgctcggttgccgccgggcgttttttattggtgagaatccaagcactag
cggcgcgccggccggcccggtgtgaaataccgcacagatgcgtaaggagaaaataccgcatcaggcgctc
ttccgcttcctcgctcactgactcgctgcgctcggtcgttcggctgcggcgagcggtatcagctcactca
aaggcggtaatacggttatccacagaatcaggggataacgcaggaaagaacatgtgagcaaaaggccagc
aaaaggccaggaaccgtaaaaaggccgcgttgctggcgtttttccataggctccgcccccctgacgagca
tcacaaaaatcgacgctcaagtcagaggtggcgaaacccgacaggactataaagataccaggcgtttccc
cctggaagctccctcgtgcgctctcctgttccgaccctgccgcttaccggatacctgtccgcctttctcc
cttcgggaagcgtggcgctttctcatagctcacgctgtaggtatctcagttcggtgtaggtcgttcgctc
caagctgggctgtgtgcacgaaccccccgttcagcccgaccgctgcgccttatccggtaactatcgtctt
gagtccaacccggtaagacacgacttatcgccactggcagcagccactggtaacaggattagcagagcga
ggtatgtaggcggtgctacagagttcttgaagtggtggcctaactacggctacactagaaggacagtatt
tggtatctgcgctctgctgaagccagttaccttcggaaaaagagttggtagctcttgatccggcaaacaa
accaccgctggtagcggtggtttttttgtttgcaagcagcagattacgcgcagaaaaaaaggatctcaag
aagatcctttgatcttttctacggggtctgacgctcagtggaacgaaaactcacgttaagggattttggt
catgagattatcaaaaaggatcttcacctagatccttttaaaggccggccgcggccgccatcggcatttt
cttttgcgtttttatttgttaactgttaattgtccttgttcaaggatgctgtctttgacaacagatgttt
tcttgcctttgatgttcagcaggaagctcggcgcaaacgttgattgtttgtctgcgtagaatcctctgtt
tgtcatatagcttgtaatcacgacattgtttcctttcgcttgaggtacagcgaagtgtgagtaagtaaag
gttacatcgttaggatcaagatccatttttaacacaaggccagttttgttcagcggcttgtatgggccag
ttaaagaattagaaacataaccaagcatgtaaatatcgttagacgtaatgccgtcaatcgtcatttttga
tccgcgggagtcagtgaacaggtaccatttgccgttcattttaaagacgttcgcgcgttcaatttcatct
gttactgtgttagatgcaatcagcggtttcatcacttttttcagtgtgtaatcatcgtttagctcaatca
taccgagagcgccgtttgctaactcagccgtgcgttttttatcgctttgcagaagtttttgactttcttg
acggaagaatgatgtgcttttgccatagtatgctttgttaaataaagattcttcgccttggtagccatct
tcagttccagtgtttgcttcaaatactaagtatttgtggcctttatcttctacgtagtgaggatctctca
gcgtatggttgtcgcctgagctgtagttgccttcatcgatgaactgctgtacattttgatacgtttttcc
gtcaccgtcaaagattgatttataatcctctacaccgttgatgttcaaagagctgtctgatgctgatacg
ttaacttgtgcagttgtcagtgtttgtttgccgtaatgtttaccggagaaatcagtgtagaataaacgga
tttttccgtcagatgtaaatgtggctgaacctgaccattcttgtgtttggtcttttaggatagaatcatt
tgcatcgaatttgtcgctgtctttaaagacgcggccagcgtttttccagctgtcaatagaagtttcgccg
actttttgatagaacatgtaaatcgatgtgtcatccgcatttttaggatctccggctaatgcaaagacga
tgtggtagccgtgatagtttgcgacagtgccgtcagcgttttgtaatggccagctgtcccaaacgtccag
gccttttgcagaagagatatttttaattgtggacgaatcaaattcagaaacttgatatttttcatttttt
tgctgttcagggatttgcagcatatcatggcgtgtaatatgggaaatgccgtatgtttccttatatggct
tttggttcgtttctttcgcaaacgcttgagttgcgcctcctgccagcagtgcggtagtaaaggttaatac
tgttgcttgttttgcaaactttttgatgttcatcgttcatgtctccttttttatgtactgtgttagcggt
ctgcttcttccagccctcctgtttgaagatggcaagttagttacgcacaataaaaaaagacctaaaatat
gtaaggggtgacgccaaagtatacactttgccctttacacattttaggtcttgcctgctttatcagtaac
aaacccgcgcgatttacttttcgacctcattctattagactctcgtttggattgcaactggtctattttc
ctcttttgtttgatagaaaatcataaaaggatttgcagactacgggcctaaagaactaaaaaatctatct
gtttcttttcattctctgtattttttatagtttctgttgcatgggcataaagttgcctttttaatcacaa
ttcagaaaatatcataatatctcatttcactaaataatagtgaacggcaggtatatgtgatgggttaaaa
aggatcggcggccgctcgatttaaatc
SEQ ID NO: 18 (complete nucleotide sequence of plasmid pSacB_delta_pckA)
(artificial)
atgaattttacccggattgacctgaatacctggaatcgcagggaacactttgccctttatcgtcagcagattaaatgcggattcagc
ctgaccaccaaactcgatattaccgctttgcgtaccgcactggcggagacaggttataagttttatccgctgatgatttacctgatct
cccgggctgttaatcagtttccggagttccggatggcactgaaagacaatgaacttatttactgggaccagtcagacccggtcttt
actgtctttcataaagaaaccgaaacattctctgcactgtcctgccgttattttccggatctcagtgagtttatggcaggttataatgcg
gtaacggcagaatatcagcatgataccagattgtttccgcagggaaatttaccggagaatcacctgaatatatcatcattaccgt
gggtgagttttgacgggatttaacctgaacatcaccggaaatgatgattattttgccccggtttttacgatggcaaagtttcagcagg
aaggtgaccgcgtattattacctgtttctgtacaggttcatcatgcagtctgtgatggctttcatgcagcacggtttattaatacacttca
gctgatgtgtgataacatactgaaataaattaattaattctgtatttaagccaccgtatccggcaggaatggtggctttttttttatatttt
aaccgtaatctgtaatttcgtttcagactggttcaggatgagctcgcttggactcctgttgatagatccagtaatgacctcagaactc
catctggatttgttcagaacgctcggttgccgccgggcgttttttattggtgagaatccaagcactagcggcgcgccggccggccc
ggtgtgaaataccgcacagatgcgtaaggagaaaataccgcatcaggcgctcttccgcttcctcgctcactgactcgctgcgct
cggtcgttcggctgcggcgagcggtatcagctcactcaaaggcggtaatacggttatccacagaatcaggggataacgcagg
aaagaacatgtgagcaaaaggccagcaaaaggccaggaaccgtaaaaaggccgcgttgctggcgtttttccataggctccg
cccccctgacgagcatcacaaaaatcgacgctcaagtcagaggtggcgaaacccgacaggactataaagataccaggcgtt
tccccctggaagctccctcgtgcgctctcctgttccgaccctgccgcttaccggatacctgtccgcctttctcccttcgggaagcgtg
gcgctttctcatagctcacgctgtaggtatctcagttcggtgtaggtcgttcgctccaagctgggctgtgtgcacgaaccccccgttc
agcccgaccgctgcgccttatccggtaactatcgtcttgagtccaacccggtaagacacgacttatcgccactggcagcagcca
ctggtaacaggattagcagagcgaggtatgtaggcggtgctacagagttcttgaagtggtggcctaactacggctacactagaa
ggacagtatttggtatctgcgctctgctgaagccagttaccttcggaaaaagagttggtagctcttgatccggcaaacaaaccac
cgctggtagcggtggtttttttgtttgcaagcagcagattacgcgcagaaaaaaaggatctcaagaagatcctttgatcttttctacg
gggtctgacgctcagtggaacgaaaactcacgttaagggattttggtcatgagattatcaaaaaggatcttcacctagatcctttta
aaggccggccgcggccgccatcggcattttcttttgcgtttttatttgttaactgttaattgtccttgttcaaggatgctgtctttgacaac
agatgttttcttgcctttgatgttcagcaggaagctcggcgcaaacgttgattgtttgtctgcgtagaatcctctgtttgtcatatagcttg
taatcacgacattgtttcctttcgcttgaggtacagcgaagtgtgagtaagtaaaggttacatcgttaggatcaagatccatttttaac
acaaggccagttttgttcagcggcttgtatgggccagttaaagaattagaaacataaccaagcatgtaaatatcgttagacgtaat
gccgtcaatcgtcatttttgatccgcgggagtcagtgaacaggtaccatttgccgttcattttaaagacgttcgcgcgttcaatttcat
ctgttactgtgttagatgcaatcagcggtttcatcacttttttcagtgtgtaatcatcgtttagctcaatcataccgagagcgccgtttgct
aactcagccgtgcgttttttatcgctttgcagaagtttttgactttcttgacggaagaatgatgtgcttttgccatagtatgctttgttaaat
aaagattcttcgccttggtagccatcttcagttccagtgtttgcttcaaatactaagtatttgtggcctttatcttctacgtagtgaggatct
ctcagcgtatggttgtcgcctgagctgtagttgccttcatcgatgaactgctgtacattttgatacgtttttccgtcaccgtcaaagattg
atttataatcctctacaccgttgatgttcaaagagctgtctgatgctgatacgttaacttgtgcagttgtcagtgtttgtttgccgtaatgtt
taccggagaaatcagtgtagaataaacggatttttccgtcagatgtaaatgtggctgaacctgaccattcttgtgtttggtcttttagg
atagaatcatttgcatcgaatttgtcgctgtctttaaagacgcggccagcgtttttccagctgtcaatagaagtttcgccgactttttgat
agaacatgtaaatcgatgtgtcatccgcatttttaggatctccggctaatgcaaagacgatgtggtagccgtgatagtttgcgaca
gtgccgtcagcgttttgtaatggccagctgtcccaaacgtccaggccttttgcagaagagatatttttaattgtggacgaatcaaatt
cagaaacttgatatttttcatttttttgctgttcagggatttgcagcatatcatggcgtgtaatatgggaaatgccgtatgtttccttatatg
gcttttggttcgtttctttcgcaaacgcttgagttgcgcctcctgccagcagtgcggtagtaaaggttaatactgttgcttgttttgcaaa
ctttttgatgttcatcgttcatgtctccttttttatgtactgtgttagcggtctgcttcttccagccctcctgtttgaagatggcaagttagttac
gcacaataaaaaaagacctaaaatatgtaaggggtgacgccaaagtatacactttgccctttacacattttaggtcttgcctgcttt
atcagtaacaaacccgcgcgatttacttttcgacctcattctattagactctcgtttggattgcaactggtctattttcctcttttgtttgata
gaaaatcataaaaggatttgcagactacgggcctaaagaactaaaaaatctatctgtttcttttcattctctgtattttttatagtttctgtt
gcatgggcataaagttgcctttttaatcacaattcagaaaatatcataatatctcatttcactaaataatagtgaacggcaggtatat
gtgatgggttaaaaaggatcggcggccgctcgatttaaatctcgagggtcggtaaaaatccgatacatccatgttttagagaaca
gagagtaggagaaattttcgattttattatgctcaatccctaaaaagattgttctccctttcgggttgttggaaaacgccaacattcaa
aaagtagcacttttgtaaccgcacttttgaggtatttaaatgaaaaaacatttcacccgctccatccaaacattgcttgtaacggca
accgcattcttctcaacctccctgcttgcagcgaccaaacagctgtacatctataactggaccgattacattccttcggatttaatttc
taaattcaccaaagaaaccggtattaaagtgaattattccaccttcgaaagcaacgaagaaatgttttccaaattgaaattaaca
atcaacaagccggggtacgatcttgtttttccctcaagttattacatcggtaaaatggtgaaagaaaatatgctggcacccatcga
acacagaaaactgacgaatttcaaacaaatcccggtcaatttattaaacaaagatttcgatccgacaaataaattttctttgcctta
tgtttacggtctgacaggaatcggtattaatacctctttcgtaaatcctgacgaagtcaccggttggggcgacttatggaaagaaa
aattcaaaggcaaagtgttattaaccgccgattcccgggaagtattccatattgcactgttattagacggaaaatcgccaaacact
caaaatgaagaagaaatccgtaacgcctaccaacgtttaacaaaaatactgccaaatgtagcggcatttaactcagatacacc
ggaactaccatacattcagggtgaagtagaactcggtatgatttggaatggttcggcttatatggcggaaaaagaaaatccggc
tattaaatttatttatccgaaagaaggcgccattttctggatggataattatgcgattcctaaaaatgcccgtaacatcgagggagc
ccataaatttatcgactttatgcttcgtccggaacacgccaaaatcattatcgaacgcatgggattttccatgcctaatgaaggcgt
gaaagtattgctaaaacctgaagaccgcgtaaacccattactgttcccgccggaagaggaagtgaaaaaaggcgtatttcag
gcagatgtaggcgatgcaaccgacatttatgaaaaatattggaataaactgaaaaccaactaaacgcttactcactttaatcaa
gcctgataacttcaccaaccttcaaaaataaccatttttttaccgcacttttactttaaaaagagcggtgaaaaacaacaagtttttta
tttaaatccgtataagtaaaaggtgaagtcaaccgtcctaaagtagaaaacaatttgttatacagattaaataatttttgccgattttc
ccacggtcttttcggctattatttccgacataaaaataagccctctgaaaagagggcttaggattgaatcaaattaaccgaattaa
gatctgtcatacatcacctcataaaataaattaaaaaataataaaaactaatgtttcgcattataggacaaaagatacctaaaaa
atgttatctagatcaaattattggaaaatatatgaaaataatttttgtttaaaaagcgaacgacattagtatttttcataaaaatacgta
cattgttatccgtcgctatttatgtaataattaatacataaataattcagataactctaaaacatggaacagaaattatcaccgaagc
aaaaaggtagacctagaacttttgatagagaaaaagcgttagaatcggcgctttttgttttttggaatcaaggttatacaaatacct
caattgcggatttatgtaatgcaattaacataaatccgccaagtttatatgctgcctttggtaataaatcacaattttttattgaaatatt
agattactatcgtcgggtgtattgggatgttatctatgccaaaatggatgttgaaaaagatattcatcgggcgattcatatattcttcc
gggactctgttaacgtagtgacagtagcaaatacgcccggtggctgtttaagtgctgttgctacattaaatttatcggcggaagaa
actaaaattcaacaacacatgaaacagttaaagtccgatattttaaaacgttttgagaaccgcttaaaacgagcgattgtggata
aacaattaccgtcgcaaaccgatattccagcattagcgctagctttacaaacttatttatatggtattgccatacaagctcaagccg
gtacaagtaaagatgatttattaaaagtggcatcgaaagccggcttattactccctaaattaatttaacaaggaaatcctttatgaa
tcctattttcagtccattatttcaaccttacaccttaaataacggtgtagaaattaaaaaccgcttagtggttgccccgatgacccact
tcggttcaaatacggacggtacattgggcgagcaagaacatcgctttatatcaaatcgtgccggtgacatgggaatgtttattcttg
ccgcaaccttagtccaagatggcggtaaagcattccacggtcaaccggaagctattcacacaagccaattaccaagtttgaaa
gccactgctgatattattaaagcgcaaggtgcaaaagcaattttacaaattcatcacggtggtaaacaggcaattaccgaattatt
aaacggcaaagataaaatttcagccagcgccgacgaagaatccggtactcgagccgcaactattgaagaaatccacacttta
attgacgctttcggcaatgctgcagatcttgccattcaagcaggttttgacggtgtagaaattcacggcgcaaacaattatctgattc
agcaattctactcgggtcattcaaatcgccgtaccgatgaatggggcggttcgcgtgaaaatcgtatgcgtttcccgttagcggta
attgatgcggtagttgcggctaaaataaagcatctctagactccataggccgctttcctggctttgcttccagatgtatgctctcctcc
ggagagtaccgtgactttattttcggcacaaatacaggggtcgatggataaatacggcgatagtttcctgacggatgatccgtatg
taccggcggaagacaagctgcaaacctgtcagatggagattgatttaatggcggatgtgctgagagcaccgccccgtgaatcc
gcagaactgatccgctatgtgtttgcggatgattggccggaataaataaagccgggcttaatacagattaagcccgtatagggta
ttattactgaataccaaacagcttacggaggacggaatgttacccattgagacaaccagactgccttctgattattaatatttttcact
attaatcagaaggaataacc

1. A modified microorganism from the family of Pasteurellaceae having an increased expression and/or increased activity of the enzyme that is encoded by the alaD-gene which encodes the alanine dehydrogenase EC 1.4.1.1,
wherein the increased expression and/or activity of the alaD-gene compared to its wildtype is achieved
a) by inserting an expression construct expressing the alaD-gene into the genome of the modified microorganism,
b) by increasing the copy number of the alaD-gene,
c) a stronger promotor compared to the wildtype of the alaD-gene,
d) by increasing the activity of genes upregulating the activity of the alaD-gene or by decreasing the activity of genes down-regulating the activity of the alaD-gene.

2. The modified microorganism according to claim 1 having a 16S rDNA of SEQ ID NO: 1 or a sequence, which shows a sequence identity of at least 96% with SEQ ID NO: 1 and/or having a 23S rDNA of SEQ ID NO: 2 or a sequence, which shows a sequence identity of at least 96% with SEQ ID NO: 2.

3. The modified microorganism according claim 1, wherein the modified microorganism belongs to the genus Basfia.

4. The modified microorganism according to claim 3, wherein the modified microorganism belongs to the species Basfia succinicproducens.

5. The modified microorganism according to claim 4, wherein the wildtype from which the modified microorganism has been derived is Basfia succiniciproducens strain DD1 as deposited under DSM 18541 with the DSMZ, Germany.

6. The modified microorganism according to claim 1, wherein the alaD-gene comprises a nucleic acid selected from the group consisting of:
a) nucleic acid having the nucleotide sequence of SEQ ID NO: 3;
b) nucleic acid encoding the amino acid sequence of SEQ ID NO: 4;
c) nucleic acids which are at least 80% identical to the nucleic acid of a) or b), the identity being the identity over the total length of the nucleic acids of a) or b); and
d) nucleic acids encoding an amino acid sequence which is at least 60% identical to the amino acid sequence encoded by the nucleic acid of a) or b), the identity being the identity over the total length of amino acid sequence encoded by the nucleic acids of a) or b)

7. The modified microorganism according to claim 1, wherein the microorganism further has
a) a reduced pyruvate formate lyase activity,
b) a reduced lactate dehydrogenase activity,
c) a reduced phosphenolpyruvate carboxylase activity or
d) any combination thereof.

8. The modified microorganism according to claim 1, wherein the microorganism comprises:
a) a deletion of the ldhA-gene or at least a part thereof, a deletion of a regulatory element of the ldhA-gene or at least a part thereof or an introduction of at least one deleterious mutation into the ldhA-gene;
b) a deletion of the pflD-gene or at least a part thereof, a deletion of a regulatory element of the pflD-gene or at least a part thereof or an introduction of at least one deleterious mutation into the pflD-gene;
c) a deletion of the pflA-gene or at least a part thereof, a deletion of a regulatory element of the pflA-gene or at least a part thereof or an introduction of at least one deleterious mutation into the pflA-gene;
d) a deletion of the pckA-gene or at least a part thereof, a deletion of a regulatory element of the pckA-gene or at least a part thereof or an introduction of at least one deleterious mutation into the pckA-gene; or
e) any combination thereof.

9. The modified microorganism according to claim 8, wherein the ldhA-gene comprises a nucleic acid selected from the group consisting of:
a) nucleic acid having the nucleotide sequence of SEQ ID NO: 5;
b) nucleic acid encoding the amino acid sequence of SEQ ID NO: 6;
c) nucleic acid which are at least 80% identical to the nucleic acid of a) or b), the identity being the identity over the total length of the nucleic acids of a) or b); and
d) nucleic acids encoding an amino acid sequence which is at least 80% identical to the amino acid sequence encoded by the nucleic acid of a) or b), the identity being the identity over the total length of amino acid sequence encoded by the nucleic acids of a) or b).

10. The modified microorganism according to claim 8,
wherein the pflD-gene comprises a nucleic acid selected from the group consisting of:
a) nucleic acid having the nucleotide sequence of SEQ ID NO: 7;
b) nucleic acid encoding the amino acid sequence of SEQ ID NO: 8;
c) nucleic acids which are at least 80% identical to the nucleic acid of a) or b), the identity being the identity over the total length of the nucleic acids of a) or b); and
d) nucleic acids encoding an amino acid sequence which is at least 80% identical to the amino acid sequence encoded by the nucleic acid of a) or b), the identity being the identity over the total length of amino acid sequence encoded by the nucleic acids of a) or b).

11. The modified microorganism according to claim 8,
wherein the pflA-gene comprises a nucleic acid selected from the group consisting of:
a) nucleic acid having the nucleotide sequence of SEQ ID NO: 9;
b) nucleic acid encoding the amino acid sequence of SEQ ID NO: 10;
c) nucleic acids which are at least 80% identical to the nucleic acid of a) or b), the identity being the identity over the total length of the nucleic acids of a) or b); and
d) nucleic acids encoding an amino acid sequence which is at least 80% identical to the amino acid sequence encoded by the nucleic acid of a) or b), the identity being the identity over the total length of amino acid sequence encoded by the nucleic acids of a) or b).

12. The modified microorganism according to claim 8, wherein the pckA-gene comprises:
a) nucleic acid having the nucleotide sequence of SEQ ID NO: 11;
b) nucleic acid encoding the amino acid sequence of SEQ ID NO: 12;
c) nucleic acid which are at least 80% identical to the nucleic acid of a) or b), the identity being the identity over the total length of the nucleic acids of a) or b); and
d) nucleic acids encoding an amino acid sequence which is at least 80% identical to the amino acid sequence encoded by the nucleic acid of a) or b), the identity being the identity over the total length of amino acid sequence encoded by the nucleic acids of a) or b).

13. A method of producing alanine comprising:
I) cultivating the modified microorganism according to claim 1 under suitable culture conditions in a culture medium to allow the modified microorganism to produce alanine, thereby obtaining a fermentation broth comprising alanine;
II) recovering alanine from the fermentation broth obtained in process step I).

14. The method according to claim 13, wherein the culture medium comprises as assimilable carbon source of glucose, sucrose, xylose, arabinose and/or glycerol.

15. The method according to claim 13, wherein the cultivation of the modified microorganism is performed under anaerobic or microaerobic conditions.

16. The method according to claim 13, wherein the process further comprises the process step:
conversion of alanine contained in the fermentation broth obtained in process step I) or conversion of the recovered alanine obtained in process step II) into a secondary organic product being different from alanine by at least one chemical reaction.

17. (canceled)


SEQUENCE LISTING (18 sequences)

SEQ ID NO: 1 - DNA, 1517 bp, Basfia succiniciproducens
tttgatcctggctcagattgaacgctggcggcaggcttaacacatgcaagtcgaacggtagcgggaggaaagcttgctttctttgccgacgagtggcggacgggtgagtaatgcttggggatctggcttatggagggggataacgacgggaaactgtcgctaataccgcgtaatatcttcggattaaagggtgggactttcgggccacccgccataagatgagcccaagtgggattaggtagttggtggggtaaaggcctaccaagccgacgatctctagctggtctgagaggatgaccagccacactggaactgagacacggtccagactcctacgggaggcagcagtggggaatattgcacaatggggggaaccctgatgcagccatgccgcgtgaatgaagaaggccttcgggttgtaaagttctttcggtgacgaggaaggtgtttgttttaataggacaagcaattgacgttaatcacagaagaagcaccggctaactccgtgccagcagccgcggtaatacggagggtgcgagcgttaatcggaataactgggcgtaaagggcatgcaggcggacttttaagtgagatgtgaaagccccgggcttaacctgggaattgcatttcagactgggagtctagagtactttagggaggggtagaattccacgtgtagcggtgaaatgcgtagagatgtggaggaataccgaaggcgaaggcagccccttgggaagatactgacgctcatatgcgaaagcgtggggagcaaacaggattagataccctggtagtccacgcggtaaacgctgtcgatttggggattgggctttaggcctggtgctcgtagctaacgtgataaatcgaccgcctggggagtacggccgcaaggttaaaactcaaatgaattgacgggggcccgcacaagcggtggagcatgtggtttaattcgatgcaacgcgaagaaccttacctactcttgacatccagagaatcctgtagagatacgggagtgccttcgggagctctgagacaggtgctgcatggctgtcgtcagctcgtgttgtgaaatgttgggttaagtcccgcaacgagcgcaacccttatcctttgttgccagcatgtaaagatgggaactcaaaggagactgccggtgacaaaccggaggaaggtggggatgacgtcaagtcatcatggcccttacgagtagggctacacacgtgctacaatggtgcatacagagggcggcgataccgcgaggtagagcgaatctcagaaagtgcatcgtagtccggattggagtctgcaactcgactccatgaagtcggaatcgctagtaatcgcaaatcagaatgttgcggtgaatacgttcccgggccttgtacacaccgcccgtcacaccatgggagtgggttgtaccagaagtagatagcttaaccttcggggggggcgtttaccacggtatgattcatgactggggtgaagtcgtaacaaggtaaccgtaggggaacctgcgg

SEQ ID NO: 2 - DNA, 3008 bp, Basfia succiniciproducens
agtaataacgaacgacacaggtataagaatacttgaggttgtatggttaagtgactaagcgtacaaggtggatgccttggcaatcagaggcgaagaaggacgtgctaatctgcgaaaagcttgggtgagttgataagaagcgtctaacccaagatatccgaatggggcaacccagtagatgaagaatctactatcaataaccgaatccataggttattgaggcaaaccgggagaactgaaacatctaagtaccccgaggaaaagaaatcaaccgagattacgtcagtagcggcgagcgaaagcgtaagagccggcaagtgatagcatgaggattagaggaatcggctgggaagccgggcggcacagggtgatagccccgtacttgaaaatcattgtgtggtactgagcttgcgagaagtagggcgggacacgagaaatcctgtttgaagaaggggggaccatcctccaaggctaaatactcctgattgaccgatagtgaaccagtactgtgaaggaaaggcgaaaagaaccccggtgaggggagtgaaatagaacctgaaaccttgtacgtacaagcagtgggagcccgcgagggtgactgcgtaccttttgtataatgggtcagcgacttatattatgtagcgaggttaaccgaataggggagccgaagggaaaccgagtcttaactgggcgtcgagttgcatgatatagacccgaaacccggtgatctagccatgggcaggttgaaggttgggtaacactaactggaggaccgaaccgactaatgttgaaaaattagcggatgacctgtggctgggggtgaaaggccaatcaaaccgggagatagctggttctccccgaaatctatttaggtagagccttatgtgaataccttcgggggtagagcactgtttcggctagggggccatcccggcttaccaacccgatgcaaactgcgaataccgaagagtaatgcataggagacacacggcgggtgctaacgttcgtcgtggagagggaaacaacccagaccgccagctaaggtcccaaagtttatattaagtgggaaacgaagtgggaaggcttagacagctaggatgttggcttagaagcagccatcatttaaagaaagcgtaatagctcactagtcgagtcggcctgcgcggaagatgtaacggggctcaaatatagcaccgaagctgcggcatcaggcgtaagcctgttgggtaggggagcgtcgtgtaagcggaagaaggtggttcgagagggctgctggacgtatcacgagtgcgaatgctgacataagtaacgataaaacgggtgaaaaacccgttcgccggaagaccaagggttcctgtccaacgttaatcggggcagggtgagtcggcccctaaggcgaggctgaagagcgtagtcgatgggaaacgggttaatattcccgtacttgttataattgcgatgtggggacggagtaggttaggttatcgacctgttggaaaaggtcgtttaagttggtaggtggagcgtttaggcaaatccggacgcttatcaacaccgagagatgatgacgaggcgctaaggtgccgaagtaaccgataccacacttccaggaaaagccactaagcgtcagattataataaaccgtactataaaccgacacaggtggtcaggtagagaatactcaggcgcttgagagaactcgggtgaaggaactaggcaaaatagcaccgtaacttcgggagaaggtgcgccggcgtagattgtagaggtatacccttgaaggttgaaccggtcgaagtgacccgctggctgcaactgtttattaaaaacacagcactctgcaaacacgaaagtggacgtatagggtgtgatgcctgcccggtgctggaaggttaattgatggcgttatcgcaagagaagcgcctgatcgaagccccagtaaacggcggccgtaactataacggtcctaaggtagcgaaattccttgtcgggtaagttccgacctgcacgaatggcataatgatggccaggctgtctccacccgagactcagtgaaattgaaatcgccgtgaagatgcggtgtacccgcggctagacggaaagaccccgtgaacctttactatagcttgacactgaaccttgaattttgatgtgtaggataggtgggaggctttgaagcggtaacgccagttatcgtggagccatccttgaaataccaccctttaacgtttgatgttctaacgaagtgcccggaacgggtactcggacagtgtctggtgggtagtttgactggggcggtctcctcccaaagagtaacggaggagcacgaaggtttgctaatgacggtcggacatcgtcaggttagtgcaatggtataagcaagcttaactgcgagacggacaagtcgagcaggtgcgaaagcaggtcatagtgatccggtggttctgaatggaagggccatcgctcaacggataaaaggtactccggggataacaggctgataccgcccaagagttcatatcgacggcggtgtttggcacctcgatgtcggctcatcacatcctggggctgaagtaggtcccaagggtatggctgttcgccatttaaagtggtacgcgagctgggtttaaaacgtcgtgagacagtttggtccctatctgccgtgggcgttggagaattgagaggggctgctcctagtacgagaggaccggagtggacgcatcactggtgttccggttgtgtcgccagacgcattgccgggtagctacatgcggaagagataagtgctgaaagcatctaagcacgaaacttgcctcgagatgagttctcccagtatttaatactgtaagggttgttggagacgacgacgtagataggccgggtgtgtaagcgttgcgagacgttgagctaaccggtactaattgcccgagaggcttagccatacaacgctcaagtgtttttggtagtgaaagttattacggaataagtaagtagtcagggaatcggct

SEQ ID NO: 3 - DNA, 1119 bp, Artificial sequence
  OTHER INFORMATION Geobacillus stearothermophilus sequence optimized for E.Coli
atgaaaattggcatccctaaagagattaagaacaatgaaaaccgtgtagcaatcaccccggcaggtgttatgactctggttaaagcgggccacgatgtgtacgtcgaaaccgaagcgggtgccggcagcggcttcagcgacagcgagtatgagaaggcgggtgcggttattgtgactaaggcggaggacgcttgggcagccgaaatggttctgaaggtgaaagaaccgctggcggaggagtttcgctattttcgtccgggtctgattttgttcacctacctgcacctggctgcggccgaggcgctgaccaaggcactggtggagcagaaggttgttggcatcgcgtacgaaacggttcaactggcgaatggttccctgccgctgctgacccctatgtctgaagttgcgggtcgcatgagcgttcaagtcggcgctcagtttctggagaaaccgcacggtggcaagggcattttgctgggtggtgttccgggtgtccgccgtggtaaagtgacgatcattggcggtggtacggccggtacgaacgcggccaagattgccgtaggtctgggtgcagatgtgaccattctggacatcaacgcggaacgtttgcgtgagctggacgatctgtttggcgaccaagtcaccaccctgatgagcaacagctaccacatcgcggagtgcgtccgtgaaagcgatttggtcgttggtgcggtgctgatcccgggtgcaaaagccccgaaactggtgaccgaggagatggtccgtagcatgaccccgggttcggttctggtcgacgtggcaattgaccagggcggtatcttcgaaaccaccgaccgcgtcacgacccatgatgacccgacctatgtgaaacatggcgtggttcactatgcggtcgcgaatatgccgggtgcagtgccgcgcacgtccacgttcgcgctgacgaacgtgacgattccatacgctctgcagatcgccaataagggctatcgtgcggcgtgtctggataatccggcattgctgaaaggcatcaataccctggatggtcatatcgtttacgaggctgtggctgcagcacacaacatgccgtacactgatgtccatagcttgctgcaaggctaa

SEQ ID NO: 4 - protein, 372 aa, Geobacillus stearothermophilus
MKIGIPKEIKNNENRVAITPAGVMTLVKAGHDVYVETEAGAGSGFSDSEYEKAGAVIVTKAEDAWAAEMVLKVKEPLAEEFRYFRPGLILFTYLHLAAAEALTKALVEQKVVGIAYETVQLANGSLPLLTPMSEVAGRMSVQVGAQFLEKPHGGKGILLGGVPGVRRGKVTIIGGGTAGTNAAKIAVGLGADVTILDINAERLRELDDLFGDQVTTLMSNSYHIAECVRESDLVVGAVLIPGAKAPKLVTEEMVRSMTPGSVLVDVAIDQGGIFETTDRVTTHDDPTYVKHGVVHYAVANMPGAVPRTSTFALTNVTIPYALQIANKGYRAACLDNPALLKGINTLDGHIVYEAVAAAHNMPYTDVHSLLQG

SEQ ID NO: 5 - DNA, 1029 bp, Basfia succiniciproducens
ttgacaaaatcagtatgtttaaataaggagctaactatgaaagttgccgtttacagtactaaaaattatgatcgcaaacatctggatttggcgaataaaaaatttaattttgagcttcatttctttgattttttacttgatgaacaaaccgcgaaaatggcggagggcgccgatgccgtctgtattttcgtcaatgatgatgcgagccgcccggtgttaacaaagttggcgcaaatcggagtgaaaattatcgctttacgttgtgccggttttaataatgtggatttggaggcggcaaaagagctgggattaaaagtcgtacgggtgcctgcgtattcgccggaagccgttgccgagcatgcgatcggattaatgctgactttaaaccgccgtatccataaggcttatcagcgtacccgcgatgcgaatttttctctggaaggattggtcggttttaatatgttcggcaaaaccgccggagtgattggtacgggaaaaatcggcttggcggctattcgcattttaaaaggcttcggtatggacgttctggcgtttgatccttttaaaaatccggcggcggaagcgttgggcgcaaaatatgtcggtttagacgagctttatgcaaaatcccatgttatcactttgcattgcccggctacggcggataattatcatttattaaatgaagcggcttttaataaaatgcgcgacggtgtaatgattattaataccagccgcggcgttttaattgacagccgggcggcaatcgaagcgttaaaacggcagaaaatcggcgctctcggtatggatgtttatgaaaatgaacgggatttgtttttcgaggataaatctaacgatgttattacggatgatgtattccgtcgcctttcttcctgtcataatgtgctttttaccggtcatcaggcgtttttaacggaagaagcgctgaataatatcgccgatgtgactttatcgaatattcaggcggtttccaaaaatgcaacgtgcgaaaatagcgttgaaggctaa

SEQ ID NO: 6 - protein, 342 aa, Basfia succiniciproducens
MTKSVCLNKELTMKVAVYSTKNYDRKHLDLANKKFNFELHFFDFLLDEQTAKMAEGADAVCIFVNDDASRPVLTKLAQIGVKIIALRCAGFNNVDLEAAKELGLKVVRVPAYSPEAVAEHAIGLMLTLNRRIHKAYQRTRDANFSLEGLVGFNMFGKTAGVIGTGKIGLAAIRILKGFGMDVLAFDPFKNPAAEALGAKYVGLDELYAKSHVITLHCPATADNYHLLNEAAFNKMRDGVMIINTSRGVLIDSRAAIEALKRQKIGALGMDVYENERDLFFEDKSNDVITDDVFRRLSSCHNVLFTGHQAFLTEEALNNIADVTLSNIQAVSKNATCENSVEG

SEQ ID NO: 7 - DNA, 2313 bp, Basfia succiniciproducens
atggctgaattaacagaagctcaaaaaaaagcatgggaaggattcgttcccggtgaatggcaaaacggcgtaaatttacgtgactttatccaaaaaaactatactccgtatgaaggtgacgaatcattcttagctgatgcgactcctgcaaccagcgagttgtggaacagcgtgatggaaggcatcaaaatcgaaaacaaaactcacgcacctttagatttcgacgaacatactccgtcaactatcacttctcacaagcctggttatatcaataaagatttagaaaaaatcgttggtcttcaaacagacgctccgttaaaacgtgcaattatgccgtacggcggtatcaaaatgatcaaaggttcttgcgaagtttacggtcgtaaattagatccgcaagtagaatttattttcaccgaatatcgtaaaacccataaccaaggcgtattcgacgtttatacgccggatattttacgctgccgtaaatcaggcgtgttaaccggtttaccggatgcttacggtcgtggtcgtattatcggtgactaccgtcgtttagcggtatacggtattgattacctgatgaaagataaaaaagcccaattcgattcattacaaccgcgtttggaagcgggcgaagacattcaggcaactatccaattacgtgaagaaattgccgaacaacaccgcgctttaggcaaaatcaaagaaatggcggcatcttacggttacgacatttccggccctgcgacaaacgcacaggaagcaatccaatggacatattttgcttatctggcagcggttaaatcacaaaacggtgcggcaatgtcattcggtcgtacgtctacattcttagatatctatatcgaacgtgacttaaaacgcggtttaatcactgaacaacaggcgcaggaattaatggaccacttagtaatgaaattacgtatggttcgtttcttacgtacgccggaatacgatcaattattctcaggcgacccgatgtgggcaaccgaaactatcgccggtatgggcttagacggtcgtccgttggtaactaaaaacagcttccgcgtattacatactttatacactatgggtacttctccggaaccaaacttaactattctttggtccgaacaattacctgaagcgttcaaacgtttctgtgcgaaagtatctattgatacttcctccgtacaatacgaaaatgatgacttaatgcgtcctgacttcaacaacgatgactatgcaatcgcatgctgcgtatcaccgatggtcgtaggtaaacaaatgcaattcttcggtgcgcgcgcaaacttagctaaaactatgttatacgcaattaacggcggtatcgatgagaaaaatggtatgcaagtcggtcctaaaactgcgccgattacagacgaagtattgaatttcgataccgtaatcgaacgtatggacagtttcatggactggttggcgactcaatatgtaaccgcattgaacatcatccacttcatgcacgataaatatgcatatgaagcggcattgatggcgttccacgatcgcgacgtattccgtacaatggcttgcggtatcgcgggtctttccgtggctgcggactcattatccgcaatcaaatatgcgaaagttaaaccgattcgcggcgacatcaaagataaagacggtaatgtcgtggcctcgaatgttgctatcgacttcgaaattgaaggcgaatatccgcaattcggtaacaatgatccgcgtgttgatgatttagcggtagacttagttgaacgtttcatgaaaaaagttcaaaaacacaaaacttaccgcaacgcaactccgacacaatctatcctgactatcacttctaacgtggtatacggtaagaaaaccggtaatactccggacggtcgtcgagcaggcgcgccattcggaccgggtgcaaacccaatgcacggtcgtgaccaaaaaggtgcggttgcttcacttacttctgtggctaaacttccgttcgcttacgcgaaagacggtatttcatataccttctctatcgtaccgaacgcattaggtaaagatgacgaagcgcaaaaacgcaaccttgccggtttaatggacggttatttccatcatgaagcgacagtggaaggcggtcaacacttgaatgttaacgttcttaaccgtgaaatgttgttagacgcgatggaaaatccggaaaaatacccgcaattaaccattcgtgtttcaggttacgcggttcgtttcaactcattaactaaagagcaacaacaagacgtcatcactcgtacgtttacacaatcaatgtaa

SEQ ID NO: 8 - protein, 770 aa, Basfia succiniciproducens
MAELTEAQKKAWEGFVPGEWQNGVNLRDFIQKNYTPYEGDESFLADATPATSELWNSVMEGIKIENKTHAPLDFDEHTPSTITSHKPGYINKDLEKIVGLQTDAPLKRAIMPYGGIKMIKGSCEVYGRKLDPQVEFIFTEYRKTHNQGVFDVYTPDILRCRKSGVLTGLPDAYGRGRIIGDYRRLAVYGIDYLMKDKKAQFDSLQPRLEAGEDIQATIQLREEIAEQHRALGKIKEMAASYGYDISGPATNAQEAIQWTYFAYLAAVKSQNGAAMSFGRTSTFLDIYIERDLKRGLITEQQAQELMDHLVMKLRMVRFLRTPEYDQLFSGDPMWATETIAGMGLDGRPLVTKNSFRVLHTLYTMGTSPEPNLTILWSEQLPEAFKRFCAKVSIDTSSVQYENDDLMRPDFNNDDYAIACCVSPMVVGKQMQFFGARANLAKTMLYAINGGIDEKNGMQVGPKTAPITDEVLNFDTVIERMDSFMDWLATQYVTALNIIHFMHDKYAYEAALMAFHDRDVFRTMACGIAGLSVAADSLSAIKYAKVKPIRGDIKDKDGNVVASNVAIDFEIEGEYPQFGNNDPRVDDLAVDLVERFMKKVQKHKTYRNATPTQSILTITSNVVYGKKTGNTPDGRRAGAPFGPGANPMHGRDQKGAVASLTSVAKLPFAYAKDGISYTFSIVPNALGKDDEAQKRNLAGLMDGYFHHEATVEGGQHLNVNVLNREMLLDAMENPEKYPQLTIRVSGYAVRFNSLTKEQQQDVITRTFTQSM

SEQ ID NO: 9 - DNA, 741 bp, Basfia succiniciproducens
atgtcggttttaggacgaattcattcatttgaaacctgcgggacagttgacgggccgggaatccgctttattttatttttacaaggctgcttaatgcgttgtaaatactgccataatagagacacctgggatttgcacggcggtaaagaaatttccgttgaagaattaatgaaagaagtggtgacctatcgccattttatgaacgcctcgggcggcggagttaccgcttccggcggtgaagctattttacaggcggaatttgtacgggactggttcagagcctgccataaagaaggaattaatacttgcttggataccaacggtttcgtccgtcatcatgatcatattattgatgaattgattgatgacacggatcttgtgttgcttgacctgaaagaaatgaatgaacgggttcacgaaagcctgattggcgtgccgaataaaagagtgctcgaattcgcaaaatatttagcggatcgaaatcagcgtacctggatccgccatgttgtagtgccgggttatacagatagtgacgaagatttgcacatgctggggaatttcattaaagatatgaagaatatcgaaaaagtggaattattaccttatcaccgtctaggcgcccataaatgggaagtactcggcgataaatacgagcttgaagatgtaaaaccgccgacaaaagaattaatggagcatgttaaggggttgcttgcaggctacgggcttaatgtgacatattag

SEQ ID NO: 10 - protein, 246 aa, Basfia succiniciproducens
MSVLGRIHSFETCGTVDGPGIRFILFLQGCLMRCKYCHNRDTWDLHGGKEISVEELMKEVVTYRHFMNASGGGVTASGGEAILQAEFVRDWFRACHKEGINTCLDTNGFVRHHDHIIDELIDDTDLVLLDLKEMNERVHESLIGVPNKRVLEFAKYLADRNQRTWIRHVVVPGYTDSDEDLHMLGNFIKDMKNIEKVELLPYHRLGAHKWEVLGDKYELEDVKPPTKELMEHVKGLLAGYGLNVTY

SEQ ID NO: 11 - DNA, 1617 bp, Basfia succiniciproducens
atgacagatcttaatcaattaactcaagaacttggtgctttaggtattcatgatgtacaagaagttgtgtataacccgagctatgaacttctttttgcggaagaaaccaaaccaggtttagacggttatgaaaaaggtactgtaactaatcaaggagcggttgctgtaaataccggtatttttaccggtcgttctccgaaagataaatatatcgttttagacgacaaaactaaagataccgtatggtggaccagcgaaaaagttaaaaacgataacaaaccaatgagtcaagatacctggaacagtttgaaaggtttagttgccgatcaactttccggtaaacgtttatttgttgttgacgcattctgtggcgcgaataaagatacgcgtttagctgttcgtgtggttactgaagttgcatggcaggcgcattttgtaacaaatatgtttatccgcccttcagcggaagaattaaaaggtttcaaacctgatttcgtggtaatgaacggtgcaaaatgtacaaatcctaactggaaagagcaaggattaaattccgaaaacttcgttgcgttcaacattacagaaggcgttcaattaatcggcggtacttggtacggcggtgaaatgaaaaaaggtatgttctcaatgatgaactacttcttaccacttcgcggtattgcatcaatgcactgttccgcaaacgttggtaaagacggcgataccgcaattttcttcggtttgtcaggtacaggtaaaactacattatcaacagatcctaaacgtcaactaatcggtgatgacgaacacggttgggacgatgaaggcgtatttaacttcgaaggtggttgctacgcgaaaaccattaacttatccgctgaaaacgagccggatatctatggcgctatcaaacgtgacgcattattggaaaacgtggtcgttttagataacggtgacgttgactatgcagacggttccaaaacagaaaatacacgtgtttcttatccgatttatcacattcaaaatatcgttaaacctgtttctaaagctggcccggcaactaaagttatcttcttgtctgccgatgcattcggtgtattaccgccggtgtctaaattaactccggaacaaaccaaatactatttcttatccggttttactgcgaaattagcgggcacagagcgtggtattacagagcctacaccaacattttctgcatgttttggtgcggctttcttaagcttgcatccgacgcaatatgccgaagtgttagtaaaacgtatgcaagaatcaggtgcggaagcgtatcttgttaatacaggttggaacggtaccggcaaacgtatctcaattaaagatacccgtggtattattgatgcaattttagacggctcaattgataaagcggaaatgggctcattaccaatcttcgatttctcaattcctaaagcattacctggtgttaaccctgcaatcttagatccgcgcgatacttatgcggataaagcgcaatgggaagaaaaagctcaagatcttgcaggtcgctttgtgaaaaactttgaaaaatataccggtacggcggaaggtcaggcattagttgctgccggtcctaaagcataa

SEQ ID NO: 12 - protein, 538 aa, Basfia succiniciproducens
MTDLNQLTQELGALGIHDVQEVVYNPSYELLFAEETKPGLDGYEKGTVTNQGAVAVNTGIFTGRSPKDKYIVLDDKTKDTVWWTSEKVKNDNKPMSQDTWNSLKGLVADQLSGKRLFVVDAFCGANKDTRLAVRVVTEVAWQAHFVTNMFIRPSAEELKGFKPDFVVMNGAKCTNPNWKEQGLNSENFVAFNITEGVQLIGGTWYGGEMKKGMFSMMNYFLPLRGIASMHCSANVGKDGDTAIFFGLSGTGKTTLSTDPKRQLIGDDEHGWDDEGVFNFEGGCYAKTINLSAENEPDIYGAIKRDALLENVVVLDNGDVDYADGSKTENTRVSYPIYHIQNIVKPVSKAGPATKVIFLSADAFGVLPPVSKLTPEQTKYYFLSGFTAKLAGTERGITEPTPTFSACFGAAFLSLHPTQYAEVLVKRMQESGAEAYLVNTGWNGTGKRISIKDTRGIIDAILDGSIDKAEMGSLPIFDFSIPKALPGVNPAILDPRDTYADKAQWEEKAQDLAGRFVKNFEKYTGTAEGQALVAAGPKA

SEQ ID NO: 13 - DNA, 4285 bp, artificial
  plasmid
tcgagaggcctgacgtcgggcccggtaccacgcgtcatatgactagttcggacctagggatatcgtcgacatcgatgctcttctgcgttaattaacaattgggatcctctagactccataggccgctttcctggctttgcttccagatgtatgctctcctccggagagtaccgtgactttattttcggcacaaatacaggggtcgatggataaatacggcgatagtttcctgacggatgatccgtatgtaccggcggaagacaagctgcaaacctgtcagatggagattgatttaatggcggatgtgctgagagcaccgccccgtgaatccgcagaactgatccgctatgtgtttgcggatgattggccggaataaataaagccgggcttaatacagattaagcccgtatagggtattattactgaataccaaacagcttacggaggacggaatgttacccattgagacaaccagactgccttctgattattaatatttttcactattaatcagaaggaataaccatgaattttacccggattgacctgaatacctggaatcgcagggaacactttgccctttatcgtcagcagattaaatgcggattcagcctgaccaccaaactcgatattaccgctttgcgtaccgcactggcggagacaggttataagttttatccgctgatgatttacctgatctcccgggctgttaatcagtttccggagttccggatggcactgaaagacaatgaacttatttactgggaccagtcagacccggtctttactgtctttcataaagaaaccgaaacattctctgcactgtcctgccgttattttccggatctcagtgagtttatggcaggttataatgcggtaacggcagaatatcagcatgataccagattgtttccgcagggaaatttaccggagaatcacctgaatatatcatcattaccgtgggtgagttttgacgggatttaacctgaacatcaccggaaatgatgattattttgccccggtttttacgatggcaaagtttcagcaggaaggtgaccgcgtattattacctgtttctgtacaggttcatcatgcagtctgtgatggctttcatgcagcacggtttattaatacacttcagctgatgtgtgataacatactgaaataaattaattaattctgtatttaagccaccgtatccggcaggaatggtggctttttttttatattttaaccgtaatctgtaatttcgtttcagactggttcaggatgagctcgcttggactcctgttgatagatccagtaatgacctcagaactccatctggatttgttcagaacgctcggttgccgccgggcgttttttattggtgagaatccaagcactagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgatttaaatc

SEQ ID NO: 14 - DNA, 8371 bp, artificial
  plasmid
tcgagtaagtgcatatgaatatgaaatacttcttgcccgccgtgtttgttacaattgacaattaaacggtagccgtcttccgcaataccttccagtttggcaattttagcggcagtaataaataagcgccctaatacggcttcatcttctgcggttacgtcgtttactgtcggaatcaatttattcggaataattaaaatatgagtttttgcctgcggcgcaatatcgcgaaatgcggtgacaagatcgtcttgatatataatgtcggcgggaatttctttacgaataattttactgaaaattgtttcttctgccattttgtgtttccttatttttgggaaaaatctaccgcactttttatcagaaatcagcttaaatagcaatttatctcgtaaaccaaaggaataaatccacaccctttataatggtattattactctatttgggtaattttgatttaggtcaaaaaatctgtaaaaggtgatatggatcactcaaattagctattatctaatttatgaatcttttataatccccccgttaaataatattcaacaattttggattttttaatctatcatttatgctttaaggcagttctactcatttccgagtagttttattactaaggaaagctcaatgaaatcggaagattttaaattggcttggatggcttcgccaaccgagatggctcaaaccgggttagacgtcggcgtttataaagctacgaaaaaacaagcctattcatttttatcggcgatctctgccggtatgtttattgctcttgcattcgttttttatacaacaactcaaacagcctctgcgggagcgccttggggattaactaaactggtcggcggtttggtgttctctctcggggtaattatggtggtggtttgcggctgtgaactatttacttcatcaactttatcgactattgcccgctttgagagtaaaattacaacaattcagatgttacgtaactggattgtggtttatttcggtaattttgtcggcggtttatttattgttgcattaatttggttttccggtcagatcatggcggcaaacggtcagtggggattaaccattttaaatacggcacaacataaaatagaacatacctggattgaagccttctgtttaggtattctttgcaacattatggtatgtattgccgtttggatggcctatgccggcaaaactctaacggataaagcttttattatgatcctgccgatcgggttatttgtcgcttcaggctttgaacactgcgtagcaaatatgtttatgatccctatgggcatggtaattgcaaatttcgcatcgccggaattctggcaggcaacgggtttaaatgccgagcagtttgcaaatttagatatgtaccatttagtaattaaaaatttaattcctgttactttaggtaacatcgtcggtggtggtgtttgcattggtctaatgcaatggtttaccagtcgtccacattagttgggtgagagtgacggcaaatccgccgtcatccttgcaaggtttcaatcttatcaatactagaaaagaaggaagtattaaaaatgaaaattggcatccctaaagagattaagaacaatgaaaaccgtgtagcaatcaccccggcaggtgttatgactctggttaaagcgggccacgatgtgtacgtcgaaaccgaagcgggtgccggcagcggcttcagcgacagcgagtatgagaaggcgggtgcggttattgtgactaaggcggaggacgcttgggcagccgaaatggttctgaaggtgaaagaaccgctggcggaggagtttcgctattttcgtccgggtctgattttgttcacctacctgcacctggctgcggccgaggcgctgaccaaggcactggtggagcagaaggttgttggcatcgcgtacgaaacggttcaactggcgaatggttccctgccgctgctgacccctatgtctgaagttgcgggtcgcatgagcgttcaagtcggcgctcagtttctggagaaaccgcacggtggcaagggcattttgctgggtggtgttccgggtgtccgccgtggtaaagtgacgatcattggcggtggtacggccggtacgaacgcggccaagattgccgtaggtctgggtgcagatgtgaccattctggacatcaacgcggaacgtttgcgtgagctggacgatctgtttggcgaccaagtcaccaccctgatgagcaacagctaccacatcgcggagtgcgtccgtgaaagcgatttggtcgttggtgcggtgctgatcccgggtgcaaaagccccgaaactggtgaccgaggagatggtccgtagcatgaccccgggttcggttctggtcgacgtggcaattgaccagggcggtatcttcgaaaccaccgaccgcgtcacgacccatgatgacccgacctatgtgaaacatggcgtggttcactatgcggtcgcgaatatgccgggtgcagtgccgcgcacgtccacgttcgcgctgacgaacgtgacgattccatacgctctgcagatcgccaataagggctatcgtgcggcgtgtctggataatccggcattgctgaaaggcatcaataccctggatggtcatatcgtttacgaggctgtggctgcagcacacaacatgccgtacactgatgtccatagcttgctgcaaggctaattgagagtttgtcttattgcttaataaattccgcctcaataggcggaatttttttgttttaattcccctgattaaagcggataaaagtgcggtagttttttgcgaagatttgactattctctgaaaaaaacgaaattctttgctataatcttcttgctatattttgttgattatttaagggcatattatgtcggttttaggacgaattcattcatttgaaacctgcgggacagttgacgggccgggaatccgctttattttatttttacaaggctgcttaatgcgttgtaaatactgccataatagagacacctgggatttgcacggcggtaaagaaatttccgttgaagaattaatgaaagaagtggtgacctatcgccattttatgaacgcctcgggcggcggagttaccgcttccggcggtgaagctattttacaggcggaatttgtacgggactggttcagagcctgccataaagaaggaattaatacttgcttggataccaacggtttcgtccgtcatcatgatcatattattgatgaattgattgatgacacggatcttgtgttgcttgacctgaaagaaatgaatgaacgggttcacgaaagcctgattggcgtgccgaataaaagagtgctcgaattcgcaaaatatttagcggatcgaaatcagcgtacctggatccgccatgttgtagtgccgggttatacagatagtgacgaagatttgcacatgctggggaatttcattaaagatatgaagaatatcgaaaaagtggaattattaccttatcaccgtctaggcgcccataaatgggaagtactcggcgataaatacgagcttgaagatgtaaaaccgccgacaaaagaattaatggagcatgttaaggggttgcttgcaggctacgggcttaatgtgacatattagaagaaataaaaaaaccgtcgtaaacattatgacggtttttttgtcactatttttcagaggagttaagccgggggtgttgtaaaagtgcggtagctttttgttgttttttctgttccctgcgcttttggaaaaagcggcttaacttctgactgcattgatcctgtaagacaccgcttgtgatctcaaccccatgattcattttataatcctcaaaaaaatgaaatctggaacccaccgcaccggttttgtaatcggacgccccgaataccaagcgtttgattcggctgtgtaaaatcgcgccggcgcacatggtgcagggttctaaagtcacgtataaagtggtattgagcaggcggtaattttggattttctgcgcggcgttacgcaacgcaataatttcggcatgggcggtgggatccgagttcacaatagagaggttccagccttcaccaatgatattgccccgttcatccaccaatacggcacctacgggaatttcccctaaagcttccgccttgtcggcaaggaaaagagctcgattcatcattttttcgtcaaagctaatttgttgatctagactccataggccgctttcctggctttgcttccagatgtatgctctcctccggagagtaccgtgactttattttcggcacaaatacaggggtcgatggataaatacggcgatagtttcctgacggatgatccgtatgtaccggcggaagacaagctgcaaacctgtcagatggagattgatttaatggcggatgtgctgagagcaccgccccgtgaatccgcagaactgatccgctatgtgtttgcggatgattggccggaataaataaagccgggcttaatacagattaagcccgtatagggtattattactgaataccaaacagcttacggaggacggaatgttacccattgagacaaccagactgccttctgattattaatatttttcactattaatcagaaggaataaccatgaattttacccggattgacctgaatacctggaatcgcagggaacactttgccctttatcgtcagcagattaaatgcggattcagcctgaccaccaaactcgatattaccgctttgcgtaccgcactggcggagacaggttataagttttatccgctgatgatttacctgatctcccgggctgttaatcagtttccggagttccggatggcactgaaagacaatgaacttatttactgggaccagtcagacccggtctttactgtctttcataaagaaaccgaaacattctctgcactgtcctgccgttattttccggatctcagtgagtttatggcaggttataatgcggtaacggcagaatatcagcatgataccagattgtttccgcagggaaatttaccggagaatcacctgaatatatcatcattaccgtgggtgagttttgacgggatttaacctgaacatcaccggaaatgatgattattttgccccggtttttacgatggcaaagtttcagcaggaaggtgaccgcgtattattacctgtttctgtacaggttcatcatgcagtctgtgatggctttcatgcagcacggtttattaatacacttcagctgatgtgtgataacatactgaaataaattaattaattctgtatttaagccaccgtatccggcaggaatggtggctttttttttatattttaaccgtaatctgtaatttcgtttcagactggttcaggatgagctcgcttggactcctgttgatagatccagtaatgacctcagaactccatctggatttgttcagaacgctcggttgccgccgggcgttttttattggtgagaatccaagcactagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgatttaaatc

SEQ ID NO: 15 - DNA, 7074 bp, artificial
  plasmid
tcgagaggcctgacgtcgggcccggtaccacgcgtcatatgactagttcggacctagggatgggtcagcctgaacgaaccgcacttgtatgtaggtagttttgaccgcccgaatattcgttataccttggtggaaaaattcaaaccgatggagcaattatacaattttgtggcggcgcaaaaaggtaaaagcggtatcgtctattgcaacagccgtagcaaagtggagcgcattgcggaagccctgaagaaaagaggcatttccgcagccgcttatcatgcgggcatggagccgtcgcagcgggaagcggtgcaacaggcgtttcaacgggataatattcaagtggtggtggcgaccattgcttttggtatggggatcaacaaatctaatgtgcgttttgtggcgcattttgatttatctcgcagcattgaggcgtattatcaggaaaccgggcgcgcggggcgggacgacctgccggcggaagcggtactgttttacgagccggcggattatgcctggttgcataaaattttattggaagagccggaaagcccgcaacgggatattaaacggcataagctggaagccatcggcgaatttgccgaaagccagacctgccgtcgtttagtgctgttaaattatttcggcgaaaaccgccaaacgccatgtaataactgtgatatctgcctcgatccgccgaaaaaatatgacggattattagacgcgcagaaaatcctttcgaccatttatcgcaccgggcaacgtttcggcacgcaatacgtaatcggcgtaatgcgcggtttgcagaatcagaaaataaaagaaaatcaacatgatgagttgaaagtctacggaattggcaaagataaaagcaaagaatactggcaatcggtaattcgtcagctgattcatttgggctttgtgcaacaaatcatcagcgatttcggcatggggaccagattacagctcaccgaaagcgcgcgtcccgtgctgcgcggcgaagtgtctttggaactggccatgccgagattatcttccattaccatggtacaggctccgcaacgcaatgcggtaaccaactacgacaaagatttatttgcccgcctgcgtttcctgcgcaaacagattgccgacaaagaaaacattccgccttatattgtgttcagtgacgcgaccttgcaggaaatgtcgttgtatcagccgaccagcaaagtggaaatgctgcaaatcaacggtgtcggcgccatcaaatggcagcgcttcggacagccttttatggcgattattaaagaacatcaggctttgcgtaaagcgggtaagaatccgttggaattgcaatcttaaaatttttaactttttgaccgcacttttaaggttagcaaattccaataaaaagtgcggtgggttttcgggaatttttaacgcgctgatttcctcgtcttttcaatttyttcgyctccatttgttcggyggttgccggatcctttcttgactgagatccataagagagtagaatagcgccgcttatatttttaatagcgtacctaatcgggtacgctttttttatgcggaaaatccatatttttctaccgcactttttctttaaagatttatacttaagtctgtttgattcaatttatttggaggttttatgcaacacattcaactggctcccgatttaacattcagtcgcttaattcaaggattctggcggttaaaaagctggcggaaatcgccgcaggaattgcttacattcgttaagcaaggattagaattaggcgttgatacgctggatcatgccgcttgttacggggcttttacttccgaggcggaattcggacgggcgctggcgctggataaatccttgcgcgcacagcttactttggtgaccaaatgcgggattttgtatcctaatgaagaattacccgatataaaatcccatcactatgacaacagctaccgccatattatgtggtcggcgcaacgttccattgaaaaactgcaatgcgactatttagatgtattgctgattcaccgwctttctccctgtgcggatcccgaacaaatcgcgcgggcttttgatgaactttatcaaaccggraaagtacgttatttcggggtatctaactatacgccggctaagttcgccatgttgcaatcttatgtgaatcagccgttaatcactaatcaaattgagatttcgcctcttcatcgtcaggcttttgatgacggtaccctggattttttactggaaaaacgtattcaaccgatggcatggtcgccacttgccggcggtcgtttattcaatcaggatgagaacagtcgggcggtgcaaaaaacattactcgaaatcggtgaaacgaaaggagaaacccgtttagatacattggcttatgcctggttattggcgcatccggcaaaaattatgccggttatggggtccggtaaaattgaacgggtaaaaagcgcggcggatgcgttacgaatttccttcactgaggaagaatggattaaggtttatgttgccgcacagggacgggatattccgtaacatcatccgtctaatcctgcgtatctggggaaagatgcgtcatcgtaagaggtctataatattcgtcgttttgataagggtgccatatccggcacccgttaaaatcacattgcgttcgcaacaaaattattccttacgaatagcattcacctcttttaacagatgttgaatatccgtatcggcaaaaatatcctctatatttgcggttaaacggcgccgccagttagcatattgagtgctggttcccggaatattgacgggttcggtcataccgagccagtcttcaggttggaatccccatcgtcgacatcgatgctcttctgcgttaattaacaattgggatcctctagactccataggccgctttcctggctttgcttccagatgtatgctctcctccggagagtaccgtgactttattttcggcacaaatacaggggtcgatggataaatacggcgatagtttcctgacggatgatccgtatgtaccggcggaagacaagctgcaaacctgtcagatggagattgatttaatggcggatgtgctgagagcaccgccccgtgaatccgcagaactgatccgctatgtgtttgcggatgattggccggaataaataaagccgggcttaatacagattaagcccgtatagggtattattactgaataccaaacagcttacggaggacggaatgttacccattgagacaaccagactgccttctgattattaatatttttcactattaatcagaaggaataaccatgaattttacccggattgacctgaatacctggaatcgcagggaacactttgccctttatcgtcagcagattaaatgcggattcagcctgaccaccaaactcgatattaccgctttgcgtaccgcactggcggagacaggttataagttttatccgctgatgatttacctgatctcccgggctgttaatcagtttccggagttccggatggcactgaaagacaatgaacttatttactgggaccagtcagacccggtctttactgtctttcataaagaaaccgaaacattctctgcactgtcctgccgttattttccggatctcagtgagtttatggcaggttataatgcggtaacggcagaatatcagcatgataccagattgtttccgcagggaaatttaccggagaatcacctgaatatatcatcattaccgtgggtgagttttgacgggatttaacctgaacatcaccggaaatgatgattattttgccccggtttttacgatggcaaagtttcagcaggaaggtgaccgcgtattattacctgtttctgtacaggttcatcatgcagtctgtgatggctttcatgcagcacggtttattaatacacttcagctgatgtgtgataacatactgaaataaattaattaattctgtatttaagccaccgtatccggcaggaatggtggctttttttttatattttaaccgtaatctgtaatttcgtttcagactggttcaggatgagctcgcttggactcctgttgatagatccagtaatgacctcagaactccatctggatttgttcagaacgctcggttgccgccgggcgttttttattggtgagaatccaagcactagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgatttaaatc

SEQ ID NO: 16 - DNA, 7161 bp, artificial
  plasmid
tcgagaggcctgacgtcgggcccggtaccacgcgtcatatgactagttcggacctagggatgggatcgagctcttttccttgccgacaaggcggaagctttaggggaaattcccgtaggtgccgtattggtggatgaacggggcaatatcattggtgaaggctggaacctctctattgtgaactcggatcccaccgcccatgccgaaattattgcgttgcgtaacgccgcgcagaaaatccaaaattaccgcctgctcaataccactttatacgtgactttagaaccctgcaccatgtgcgccggcgcgattttacacagccgaatcaaacgcttggtattcggggcgtccgattacaaaaccggtgcggtgggttccagatttcatttttttgaggattataaaatgaatcatggggttgagatcacaagcggtgtcttataggatcaatgcagtcagaagttaagccgctttttccaaaagcgcagggaacagaaaaaacaacaaaaagctaccgcacttttacaacacccccggcttaactcctctgaaaaatagtgacaaaaaaaccgtcataatgtttacgacggtttttttatttcttctaatatgtcacattaagcccgtagcctgcaagcaaccccttaacatgctccattaattcttttgtcggcggttttacatcttcaagctcgtatttatcgccgagtacttcccatttatgggcgcctagacggtgataaggtaataattccactttttcgatattcttcatatctttaatgaaattccccagcatgtgcaaatcttcgtcactatctgtataacccggcactacaacatggcggatccaggtacgctgatttcgatccgctaaatattttgcgaattcgagcactcttttattcggcacgccaatcaggctttcgtgaacccgttcattcatttctttcaggtcaagcaacacaagatccgtgtcatcaatcaattcatcaataatatgatcatgatgacggacgaaaccgttggtatccaagcaagtattaattccttctttatggcaggctctgaaccagtcccgtacaaattccgcctgtaaaatagcttcaccgccggaagcggtaactccgccgcccgaggcgttcataaaatggcgataggtcaccacttctttcattaattcttcaacggaaatttctttaccgccgtgcaaatcccaggtgtctctgttatggcaatatttacaacgcattaagcagccttgtaaaaataaaataaagcggattcccggcccgtcaactgtcccgcaggtttcaaatgaatgaattcgtcctaaaaccgacataatatgcccttaaataatcaacaaaatatagcaagaagattatagcaaagaatttcgtttttttcagagaatagtcaaatcttcgcaaaaaactaccgcacttttatccgctttaatcaggggaattaaaacaaaaaaattccgcctattgaggcggaatttattaagcaataagacaaactctcaattttaatacttccttcttttctagtattgataagattgaaaccttgcaaggatgacggcggatttgccgtcactctcacccaactaatgtggacgactggtaaaccattgcattagaccaatgcaaacaccaccaccgacgatgttacctaaagtaacaggaattaaatttttaattactaaatggtacatatctaaatttgcaaactgctcggcatttaaacccgttgcctgccagaattccggcgatgcgaaatttgcaattaccatgcccatagggatcataaacatatttgctacgcagtgttcaaagcctgaagcgacaaayaacccgatcggcaggatcataataaaagctttatccgttagagtyttgccggcataggccatccaaacggcaatacataccataatgttgcaaagaatacctaaacagaaggcttcaayccaggtatgttctattttatgttgtgccgtatttaaaatggttaatccccactgaccgtttgccgccatgatctgaccggaaaaccaaattaatgcaacaataaataaaccgccgacaaaattaccgaartaaaccacaatccagttacgtaacatctgaattgttgtaattttactctcaaagcgggcaatagtcgataaagttgatgaagtaaatagttcacagccgcaaaccgccaccataattaccccgagagagaacaccaaaccgccgaccagtttagttaatccccaaggcgctcccgcagaggctgtttgagttgttgtataaaaaacgaatgcaagagcaataaacataccggcagagatcgccgataaaaatgaataggcttgttttttcgtagctttataaacgccgacgtctaacccggtttgagccatctcggttggcgaagccatccaagccaatttaaaatcttccgatttcattgagctttccttagtaataaaactactcggaaatgagtagaactgccttaaagcataaatgatagattaaaaaatccaaaattgttgaatattatttaacggggggattataaaagattcataaattagataatagctaatttgagtgatccatatcaccttttacagattttttgacctaaatcaaaattacccaaatagagtaataataccattataaagggtgtggatttattcctttggtttacgagataaattgctatttaagctgatttctgataaaaagtgcggtagatttttcccaaaaataaggaaacacaaaatggcagaagaaacaattttcagtaaaattattcgtaaagaaattcccgccgacattatatatcaagacgatcttgtcaccgcatttcgcgatattgcgccgcaggcaaaaactcatattttaattattccgaataaattgattccgacagtaaacgacgtaaccgcccatcgtcgacatcgatgctcttctgcgttaattaacaattgggatcctctagactttgcttccagatgtatgctctcctccggagagtaccgtgactttattttcggcacaaatacaggggtcgatggataaatacggcgatagtttcctgacggatgatccgtatgtaccggcggaagacaagctgcaaacctgtcagatggagattgatttaatggcggatgtgctgagagcaccgccccgtgaatccgcagaactgatccgctatgtgtttgcggatgattggccggaataaataaagccgggcttaatacagattaagcccgtatagggtattattactgaataccaaacagcttacggaggacggaatgttacccattgagacaaccagactgccttctgattattaatatttttcactattaatcagaaggaataaccatgaattttacccggattgacctgaatacctggaatcgcagggaacactttgccctttatcgtcagcagattaaatgcggattcagcctgaccaccaaactcgatattaccgctttgcgtaccgcactggcggagacaggttataagttttatccgctgatgatttacctgatctcccgggctgttaatcagtttccggagttccggatggcactgaaagacaatgaacttatttactgggaccagtcagacccggtctttactgtctttcataaagaaaccgaaacattctctgcactgtcctgccgttattttccggatctcagtgagtttatggcaggttataatgcggtaacggcagaatatcagcatgataccagattgtttccgcagggaaatttaccggagaatcacctgaatatatcatcattaccgtgggtgagttttgacgggatttaacctgaacatcaccggaaatgatgattattttgccccggtttttacgatggcaaagtttcagcaggaaggtgaccgcgtattattacctgtttctgtacaggttcatcatgcagtctgtgatggctttcatgcagcacggtttattaatacacttcagctgatgtgtgataacatactgaaataaattaattaattctgtatttaagccaccgtatccggcaggaatggtggctttttttttatattttaaccgtaatctgtaatttcgtttcagactggttcaggatgagctcgcttggactcctgttgatagatccagtaatgacctcagaactccatctggatttgttcagaacgctcggttgccgccgggcgttttttattggtgagaatccaagcactagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgatttaaatc

SEQ ID NO: 17 - DNA, 5137 bp, artificial
  plasmid
tttttggtcacgaccgtgcattgggtttgcacccggtccgaatggcgcgcctgctcgacgaccgtccggagtattaccggttttcttaccgtataccacgttagaagtgatagtcaggatagattgtgtcggagttgcgttgcggtaagttttgtgtttttgaacttttttcatgaaacgttcaactaagtctaccgctaaatcatcaacacgcggatcattgttaccgaattgcggatattcgccttcaatttcgaagtcgatagcaacattcgaggccacgacattaccgtctttatctttgatgtcgccgcgaatcggtttaactttcgcatatttgattgcggataatgagtccgcagccacggaaagacccgcgataccgcaagccattgtacggaatacgtcgcgatcgtggaacgccatcaatgccgcttcatatgcatatttatcgtgcatgaagtggatgatgttcaatgcggttacatattgagtcgccaaccagtccatgaaactgtccatacgttcgattacggtatcgaaattcaatacttcgtctgtaatcggcgcagttttaggaccgacttgcataccatttttctcatcgataccgccgttaattgcgtataacatagttttagctaagtttgcgcgcgcaccgaagaattgcatttgtttacctacgaccatcggtgatacgcagcatgcgattgcatagtcatcgttgttgaagtcaggacgcattaagtcatcattttcgtattgtacggaggaagtatcaatagatactttcgcacagaaacgtttgaacgcttcaggtaattgttcggaccaaagaatagttaagtttggttccggagaagtacccatagtgtataaagtatgtaatacgcggaagctgtttttagttaccaacggacgaccgtctaagcccataccggcgatagtttcggttgccctctagactccataggccgctttcctggctttgcttccagatgtatgctctcctccggagagtaccgtgactttattttcggcacaaatacaggggtcgatggataaatacggcgatagtttcctgacggatgatccgtatgtaccggcggaagacaagctgcaaacctgtcagatggagattgatttaatggcggatgtgctgagagcaccgccccgtgaatccgcagaactgatccgctatgtgtttgcggatgattggccggaataaataaagccgggcttaatacagattaagcccgtatagggtattattactgaataccaaacagcttacggaggacggaatgttacccattgagacaaccagactgccttctgattattaatatttttcactattaatcagaaggaataaccatgaattttacccggattgacctgaatacctggaatcgcagggaacactttgccctttatcgtcagcagattaaatgcggattcagcctgaccaccaaactcgatattaccgctttgcgtaccgcactggcggagacaggttataagttttatccgctgatgatttacctgatctcccgggctgttaatcagtttccggagttccggatggcactgaaagacaatgaacttatttactgggaccagtcagacccggtctttactgtctttcataaagaaaccgaaacattctctgcactgtcctgccgttattttccggatctcagtgagtttatggcaggttataatgcggtaacggcagaatatcagcatgataccagattgtttccgcagggaaatttaccggagaatcacctgaatatatcatcattaccgtgggtgagttttgacgggatttaacctgaacatcaccggaaatgatgattattttgccccggtttttacgatggcaaagtttcagcaggaaggtgaccgcgtattattacctgtttctgtacaggttcatcatgcagtctgtgatggctttcatgcagcacggtttattaatacacttcagctgatgtgtgataacatactgaaataaattaattaattctgtatttaagccaccgtatccggcaggaatggtggctttttttttatattttaaccgtaatctgtaatttcgtttcagactggttcaggatgagctcgcttggactcctgttgatagatccagtaatgacctcagaactccatctggatttgttcagaacgctcggttgccgccgggcgttttttattggtgagaatccaagcactagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgatttaaatc

SEQ ID NO: 18 - DNA, 7198 bp, artificial
  plasmid
atgaattttacccggattgacctgaatacctggaatcgcagggaacactttgccctttatcgtcagcagattaaatgcggattcagcctgaccaccaaactcgatattaccgctttgcgtaccgcactggcggagacaggttataagttttatccgctgatgatttacctgatctcccgggctgttaatcagtttccggagttccggatggcactgaaagacaatgaacttatttactgggaccagtcagacccggtctttactgtctttcataaagaaaccgaaacattctctgcactgtcctgccgttattttccggatctcagtgagtttatggcaggttataatgcggtaacggcagaatatcagcatgataccagattgtttccgcagggaaatttaccggagaatcacctgaatatatcatcattaccgtgggtgagttttgacgggatttaacctgaacatcaccggaaatgatgattattttgccccggtttttacgatggcaaagtttcagcaggaaggtgaccgcgtattattacctgtttctgtacaggttcatcatgcagtctgtgatggctttcatgcagcacggtttattaatacacttcagctgatgtgtgataacatactgaaataaattaattaattctgtatttaagccaccgtatccggcaggaatggtggctttttttttatattttaaccgtaatctgtaatttcgtttcagactggttcaggatgagctcgcttggactcctgttgatagatccagtaatgacctcagaactccatctggatttgttcagaacgctcggttgccgccgggcgttttttattggtgagaatccaagcactagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgatttaaatctcgagggtcggtaaaaatccgatacatccatgttttagagaacagagagtaggagaaattttcgattttattatgctcaatccctaaaaagattgttctccctttcgggttgttggaaaacgccaacattcaaaaagtagcacttttgtaaccgcacttttgaggtatttaaatgaaaaaacatttcacccgctccatccaaacattgcttgtaacggcaaccgcattcttctcaacctccctgcttgcagcgaccaaacagctgtacatctataactggaccgattacattccttcggatttaatttctaaattcaccaaagaaaccggtattaaagtgaattattccaccttcgaaagcaacgaagaaatgttttccaaattgaaattaacaatcaacaagccggggtacgatcttgtttttccctcaagttattacatcggtaaaatggtgaaagaaaatatgctggcacccatcgaacacagaaaactgacgaatttcaaacaaatcccggtcaatttattaaacaaagatttcgatccgacaaataaattttctttgccttatgtttacggtctgacaggaatcggtattaatacctctttcgtaaatcctgacgaagtcaccggttggggcgacttatggaaagaaaaattcaaaggcaaagtgttattaaccgccgattcccgggaagtattccatattgcactgttattagacggaaaatcgccaaacactcaaaatgaagaagaaatccgtaacgcctaccaacgtttaacaaaaatactgccaaatgtagcggcatttaactcagatacaccggaactaccatacattcagggtgaagtagaactcggtatgatttggaatggttcggcttatatggcggaaaaagaaaatccggctattaaatttatttatccgaaagaaggcgccattttctggatggataattatgcgattcctaaaaatgcccgtaacatcgagggagcccataaatttatcgactttatgcttcgtccggaacacgccaaaatcattatcgaacgcatgggattttccatgcctaatgaaggcgtgaaagtattgctaaaacctgaagaccgcgtaaacccattactgttcccgccggaagaggaagtgaaaaaaggcgtatttcaggcagatgtaggcgatgcaaccgacatttatgaaaaatattggaataaactgaaaaccaactaaacgcttactcactttaatcaagcctgataacttcaccaaccttcaaaaataaccatttttttaccgcacttttactttaaaaagagcggtgaaaaacaacaagttttttatttaaatccgtataagtaaaaggtgaagtcaaccgtcctaaagtagaaaacaatttgttatacagattaaataatttttgccgattttcccacggtcttttcggctattatttccgacataaaaataagccctctgaaaagagggcttaggattgaatcaaattaaccgaattaagatctgtcatacatcacctcataaaataaattaaaaaataataaaaactaatgtttcgcattataggacaaaagatacctaaaaaatgttatctagatcaaattattggaaaatatatgaaaataatttttgtttaaaaagcgaacgacattagtatttttcataaaaatacgtacattgttatccgtcgctatttatgtaataattaatacataaataattcagataactctaaaacatggaacagaaattatcaccgaagcaaaaaggtagacctagaacttttgatagagaaaaagcgttagaatcggcgctttttgttttttggaatcaaggttatacaaatacctcaattgcggatttatgtaatgcaattaacataaatccgccaagtttatatgctgcctttggtaataaatcacaattttttattgaaatattagattactatcgtcgggtgtattgggatgttatctatgccaaaatggatgttgaaaaagatattcatcgggcgattcatatattcttccgggactctgttaacgtagtgacagtagcaaatacgcccggtggctgtttaagtgctgttgctacattaaatttatcggcggaagaaactaaaattcaacaacacatgaaacagttaaagtccgatattttaaaacgttttgagaaccgcttaaaacgagcgattgtggataaacaattaccgtcgcaaaccgatattccagcattagcgctagctttacaaacttatttatatggtattgccatacaagctcaagccggtacaagtaaagatgatttattaaaagtggcatcgaaagccggcttattactccctaaattaatttaacaaggaaatcctttatgaatcctattttcagtccattatttcaaccttacaccttaaataacggtgtagaaattaaaaaccgcttagtggttgccccgatgacccacttcggttcaaatacggacggtacattgggcgagcaagaacatcgctttatatcaaatcgtgccggtgacatgggaatgtttattcttgccgcaaccttagtccaagatggcggtaaagcattccacggtcaaccggaagctattcacacaagccaattaccaagtttgaaagccactgctgatattattaaagcgcaaggtgcaaaagcaattttacaaattcatcacggtggtaaacaggcaattaccgaattattaaacggcaaagataaaatttcagccagcgccgacgaagaatccggtactcgagccgcaactattgaagaaatccacactttaattgacgctttcggcaatgctgcagatcttgccattcaagcaggttttgacggtgtagaaattcacggcgcaaacaattatctgattcagcaattctactcgggtcattcaaatcgccgtaccgatgaatggggcggttcgcgtgaaaatcgtatgcgtttcccgttagcggtaattgatgcggtagttgcggctaaaataaagcatctctagactccataggccgctttcctggctttgcttccagatgtatgctctcctccggagagtaccgtgactttattttcggcacaaatacaggggtcgatggataaatacggcgatagtttcctgacggatgatccgtatgtaccggcggaagacaagctgcaaacctgtcagatggagattgatttaatggcggatgtgctgagagcaccgccccgtgaatccgcagaactgatccgctatgtgtttgcggatgattggccggaataaataaagccgggcttaatacagattaagcccgtatagggtattattactgaataccaaacagcttacggaggacggaatgttacccattgagacaaccagactgccttctgattattaatatttttcactattaatcagaaggaataacc